SEQUENCE LISTING (120 sequences)

SEQ ID NO: 1 - protein, 6 aa, Homo sapiens
MYPPPY

SEQ ID NO: 2 - protein, 4 aa, Homo sapiens
SICT

SEQ ID NO: 3 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 4 - DNA, 1997 bp, Homo sapiens
  source 1..1997: mol_type = genomic DNA
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 5 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 6 - DNA, 1887 bp, Homo sapiens
  source 1..1887: mol_type = genomic DNA
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 7 - protein, 223 aa, Mus sp.
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVTAVSLSKMLKKRSPLTTGVYVKMPPTEPECEKQFQPYFIPIN

SEQ ID NO: 8 - DNA, 1933 bp, Mus sp.
  source 1..1933: mol_type = genomic DNA
ctacacatatgtagcacgtaccttggatcaaagctgtctatataaagtccccgagtctgtgtgggttcaaacacatctcaaggcttctggatcctgttgggttttactctgctccctgaggacctcagcacatttgccccccagccatggcttgtcttggactccggaggtacaaagctcaactgcagctgccttctaggacttggccttttgtagccctgctcactcttcttttcatcccagtcttctctgaagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgacttcctcctttggatccttgtcgcagttagcttggggttgtttttttacagtttcctggtcactgctgtttctttgagcaagatgctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggatgcaaaattccaattaagcatactacaatttaaagctaaggagcatgaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagatgtttatgacttaaataatttttaaatttttcatgctgttattttctttaacaatgtataattacacgaaggtttaaacatttattcacagagctatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcctggggaggtttggctggggagcttaggacactaatccatcaggttggactcgaggtcctgcaccaactggcttggaaactagatgaggctgtcacagggctcagttgcataaaccgatggtgatggagtgtaaactgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtttgtttttttaggaagttccaatactaggaccccctacaagtactatggctcctcgaaaacacaaagttaatgccacaggaagcagcagatggtaggatgggatgcacaagagttcctgaaaactaacactgttagtgttttttttttaactcaatattttccatgaaaatgcaaccacatgtataatatttttaattaaataaaagtttcttgtgattgtttt

SEQ ID NO: 9 - protein, 142 aa, synthetic construct
MDVLVLFLCLVAFPSCVLSQVQLKESGPGLVAPSQSLSITCTVSGFSLTSYGVYWVRQPPGKGLEWLGVIWAGGTTNYNSALMSRLSISKDNSKSQVFLKMSSLQTDDTAMYYCARGPPHAMMKRGYAMDYWGQGTSVIVSS

SEQ ID NO: 10 - protein, 128 aa, synthetic construct
MDFQVQIFSFLLISASVILSRGQNVLTQSPAIMPASPGEKVTMTCSATSSITYMSWYQQKSGSSPRLLIYDTSNLASGVPVRFSGSGSGTSYSLTISRMEAEDAATYYCQQWSSYPLTFGAGTKLELK

SEQ ID NO: 11 - DNA, 384 bp, synthetic construct
  source 1..384: mol_type = other DNA
atggattttcaagtgcagattttcagcttcctgctaatcagtgcctcagtcatactgtccagaggacaaaatgttctcacccagtctccagcaatcatgcctgcatctccaggggagaaggtcaccatgacctgcagtgccacctcaagtataacttacatgtcctggtaccagcagaagtcaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaa

SEQ ID NO: 12 - protein, 123 aa, synthetic construct
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 13 - DNA, 371 bp, synthetic construct
  source 1..371: mol_type = other DNA
caggtgcagctgcaagagtcaggacctggcctggtgaagccctcacagacactgtccttgacttgcactgtctctgggttttcattaacctcatatggtgtatattgggttcgccagcctccaggaaagggtctggagtggctgggagtaatatgggctggtggtaccacaaattataattcggctctcatgtccagactgacaatcagcaaagacacatccaagaaccaagtttccttaaaactcagcagtgtgactgcagcggacacagccgtctactactgtgcccgaggccccccgcacgctatgatgaagagaggctatgctatggactactggggacaaggaaccctagtcacagtctcctcagg

SEQ ID NO: 14 - protein, 106 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKLEIK

SEQ ID NO: 15 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 16 - DNA, 355 bp, synthetic construct
  source 1..355: mol_type = other DNA
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 17 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 18 - DNA, 325 bp, synthetic construct
  source 1..325: mol_type = other DNA
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 19 - protein, 241 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 20 - protein, 244 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 21 - protein, 221 aa, synthetic construct
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 22 - protein, 213 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 482 aa, synthetic construct
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGSGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIK

SEQ ID NO: 24 - protein, 482 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 25 - DNA, 1476 bp, synthetic construct
  source 1..1476: mol_type = other DNA
caagtgcagctccaagagtccggccccggcctcgtgaaacccagccagacactgtctctgacatgcaccgtgagcggcttttctctgaccagctacggagtgtattgggtgagacaaccccccggcaagggactggagtggctgggagtgatttgggccggcggcaccaccaactacaatagcgccctcatgtctagactcaccatctccaaggacaccagcaagaaccaagtgtccctcaagctgtccagcgtcacagctgccgacaccgccgtgtactattgtgctagaggccccccccatgccatgatgaagagaggctatgccatggattactggggccaaggcacactggtgaccgtcagctccggaggaagcggcggcagcggaggctccgaaattgtgctcacccagagccccggcacactgtctctgagccccggcgaaagggccacactgagctgcagagcctcccaatccgtgggcagcagctatctggcttggtatcagcagaaacccggccaagcccctagactgctgatctatggagccttttctagagctaccggcatccccgacagattctccggcagcggcagcggcacagactttacactgacaatttctagactggaaccagaggatttcgccgtctactactgccagcagtatggaagcagcccttggacctttggccaaggcaccaaggtggagatcaagggaggaagcggaggcagcggaggcagccaagtgcagctcgtggaaagcggaggaggcgtggtgcagcccggcagatccctcagactgagctgcgccgccagcggcttcaccttcagctcctataccatgcactgggtgaggcaagcccccggcaaaggactggagtgggtcaccttcatcagctacgacggcaacaacaagtactacgccgacagcgtgaagggaaggttcaccatctctagagacaactccaagaacaccctctacctccagatgaactctctgagggccgaagacaccgccatctactactgcgctagaaccggctggctgggaccctttgactactggggacaaggcacactggtcacagtgtcctccggaggaagcggaggctccggcggcagcgatatccagatgacccaatccccttcctctctgagcgcctccgtgggagatagggtcaccattacatgtagcgccacaagcagcatcacctacatgagctggtaccagcagaaacccggaaaggcccctaagctgctcatctacgacacctccaatctggccagcggcgtgccttctagatttagcggctccggaagcggcacagattacacactgacaatcagctctctgcagccagaggacttcgccacctactactgtcagcagtggagcagctaccctctgacctttggcggcggcaccaaggtggaaatcaaacaccaccatcaccaccatcaccaccatcac

SEQ ID NO: 26 - DNA, 1476 bp, synthetic construct
  source 1..1476: mol_type = other DNA
caagtgcagctcgtggaatccggcggaggagtcgtgcagcccggcagaagcctcagactgagctgcgccgccagcggattcaccttcagcagctacaccatgcactgggtgaggcaagcccccggcaaaggactggagtgggtcacattcatctcctacgatggcaacaacaagtactacgccgacagcgtgaagggaaggtttaccatctctagagataactccaagaacaccctctacctccagatgaactctctgagagctgaggacacagccatctattactgcgctagaaccggatggctgggccctttcgactactggggacaaggcacactggtgacagtgtcctccggaggcagcggaggcagcggaggcagcgacatccagatgacccaatcccctagctctctgagcgccagcgtgggcgatagagtgaccattacatgctccgccaccagcagcatcacctacatgagctggtaccagcaaaagcccggcaaagcccccaagctgctgatctacgataccagcaatctggccagcggcgtgccttctagattttccggctccggaagcggcaccgattacacactgaccatttcctctctgcagccagaggatttcgccacctactattgccagcagtggtcctcctaccctctcacctttggcggcggaacaaaggtggagatcaaaggcggcagcggcggctccggaggcagccaagtccagctgcaagagtccggacccggactggtgaaaccttcccagacactgtctctgacatgcacagtgagcggattctccctcacaagctacggcgtgtactgggtgagacagcctcccggcaaaggactggagtggctgggcgtcatctgggctggaggcaccaccaattacaacagcgctctgatgtctagactgacaatctccaaggacaccagcaagaaccaagtgtctctgaagctgagctccgtgacagccgccgatacagccgtgtactattgtgccagaggccccccccacgctatgatgaagaggggctacgccatggactactggggccaaggcaccctcgtcacagtgagctccggaggcagcggaggaagcggcggcagcgagatcgtgctgacccagtcccccggcacactgtccctcagccccggcgaaagagccacactgagctgtagagcttcccagagcgtgggcagcagctatctggcttggtatcagcagaagcccggccaagcccccagactgctcatctacggagccttcagcagagccaccggcatccccgacagattcagcggcagcggcagcggcaccgattttaccctcaccatctccagactggagccagaggacttcgccgtgtattactgccaacagtacggcagcagcccttggacctttggacaaggcaccaaggtggaaatcaaacaccaccaccatcaccatcatcatcaccac

SEQ ID NO: 27 - protein, 216 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 28 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 503 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSKEAKEAKEAKEAKEAKEADIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 30 - protein, 503 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSKEAKEAKEAKEAKEAKEAEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 31 - protein, 478 aa, synthetic construct
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHHHHHHHHHH

SEQ ID NO: 32 - protein, 213 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 215 aa, synthetic construct; identical to SEQ ID NO: 28

SEQ ID NO: 34 - protein, 468 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 35 - protein, 215 aa, synthetic construct; identical to SEQ ID NO: 28

SEQ ID NO: 36 - protein, 213 aa, synthetic construct; identical to SEQ ID NO: 32

SEQ ID NO: 37 - protein, 459 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 38 - protein, 224 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 39 - protein, 213 aa, synthetic construct; identical to SEQ ID NO: 32

SEQ ID NO: 40 - protein, 452 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 41 - protein, 229 aa, synthetic construct
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 42 - protein, 215 aa, synthetic construct; identical to SEQ ID NO: 28

SEQ ID NO: 43 - protein, 273 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGSGGGGQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGCGGGEVAALEKEVAALEKEVAALEKEVAALEK

SEQ ID NO: 44 - protein, 266 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGSGGGGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGCGGGKVAALKEKVAALKEKVAALKEKVAALKE

SEQ ID NO: 45 - protein, 701 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFRLYSRLTVDKSRWQQGNVFSCSVMHEALHNHYTQKSLSLSPGGGGGSGGGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 46 - protein, 215 aa, synthetic construct; identical to SEQ ID NO: 28

SEQ ID NO: 47 - protein, 447 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 481 aa, synthetic construct
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGGGSEPKSQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 215 aa, synthetic construct; identical to SEQ ID NO: 28

SEQ ID NO: 50 - protein, 452 aa, synthetic construct
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 478 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSEPKSQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 213 aa, synthetic construct; identical to SEQ ID NO: 32

SEQ ID NO: 53 - protein, 477 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 481 aa, synthetic construct; identical to SEQ ID NO: 48

SEQ ID NO: 55 - protein, 22 aa, synthetic construct
MDFQVQIFSFLLISASVILSRG

SEQ ID NO: 56 - protein, 714 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 2142 bp, synthetic construct
  source 1..2142: mol_type = other DNA
caagtgcagctggtggagagcggtggcggcgtggtgcagcccggtagaagcctgagactgagctgcgccgcaagcggcttcaccttcagcagctacaccatgcactgggtgagacaagcacccggcaagggactggaatgggtcacgttcatcagctacgacggcaacaacaagtactacgccgacagcgtgaagggcagattcaccatcagccgagacaacagcaagaacaccctgtacctgcagatgaacagcctgagagccgaggacaccgccatctactattgcgctagaacgggatggctgggccccttcgattattgggggcaaggcacgttggtgaccgtctcaagcggcggaagcggaggcagcggaggatcggatatacagatgacacagtcgcctagctcactgagcgcaagcgtgggcgacagagtgaccatcacctgcagcgccacaagcagcatcacctacatgagctggtatcagcaaaaacccggcaaggcccccaagctgctgatctacgatacaagcaacctggcaagcggcgtgcctagcagattctctggcagcggcagcggcaccgactatactctcaccataagcagtctacagcctgaggatttcgccacctactattgtcagcagtggtcgagctaccccctgaccttcgggggcggcaccaaggtagagattaaaggtgggagcggcgggagtggtggcagccaagtccaactgcaagagtccggacccggtcttgtgaagccttctcagactcttagcttaacatgcaccgtgagcggcttcagccttacaagctacggcgtctactgggtacgtcaaccacctggtaagggtttagaatggctaggggtgatctgggccggcggcaccaccaactacaacagcgccctgatgagcagactgacgatctctaaggacactagcaaaaaccaagtgagcctcaagctgagcagcgtgaccgccgccgacaccgccgtatactactgtgcaagaggccccccccacgccatgatgaagagaggctacgccatggattactgggggcaaggcactctagtgaccgtgagtagtggcgggagcggtgggagcggcggttccgaaatcgtgctgacacagagccctggcactctgtctctgagtcccggcgagagagctaccctgagctgcagagcatctcagagcgtgggcagcagctacctggcctggtatcagcaaaagcccggccaagcccctagacttctgatctacggcgcattcagcagagccaccggcatccccgacagattcagtggatctggcagcggaacggacttcaccctaacaatcagccgtctggaacccgaagactttgcggtgtattactgtcaacagtacggtagcagcccctggaccttcggccaaggcacaaaggtggagatcaaaggcggttccggtggcgacaaaacacacacctgtcccccctgccccgcccctgagctgttaggtggtcctagcgtgttcctgttcccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagacgagctgaccaagaaccaagtgtcgctaacctgtttggtgaagggcttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgtcgctgagccccggaaaa

SEQ ID NO: 58 - protein, 714 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 2142 bp, synthetic construct
  source 1..2142: mol_type = other DNA
caagtgcagctggtggagagcggtggcggcgtggtgcagcccggtagaagcctgagactgagctgcgccgcaagcggcttcaccttcagcagctacaccatgcactgggtgagacaagcacccggcaagggactggaatgggtcacgttcatcagctacgacggcaacaacaagtactacgccgacagcgtgaagggcagattcaccatcagccgagacaacagcaagaacaccctgtacctgcagatgaacagcctgagagccgaggacaccgccatctactattgcgctagaacgggatggctgggccccttcgattattgggggcaaggcacgttggtgaccgtctcaagcggcggaagcggaggcagcggaggatcggatatacagatgacacagtcgcctagctcactgagcgcaagcgtgggcgacagagtgaccatcacctgcagcgccacaagcagcatcacctacatgagctggtatcagcaaaaacccggcaaggcccccaagctgctgatctacgatacaagcaacctggcaagcggcgtgcctagcagattctctggcagcggcagcggcaccgactatactctcaccataagcagtctacagcctgaggatttcgccacctactattgtcagcagtggtcgagctaccccctgaccttcgggggcggcaccaaggtagagattaaaggtgggagcggcgggagtggtggcagccaagtccaactgcaagagtccggacccggtcttgtgaagccttctcagactcttagcttaacatgcaccgtgagcggcttcagccttacaagctacggcgtctactgggtacgtcaaccacctggtaagggtttagaatggctaggggtgatctgggccggcggcaccaccaactacaacagcgccctgatgagcagactgacgatctctaaggacactagcaaaaaccaagtgagcctcaagctgagcagcgtgaccgccgccgacaccgccgtatactactgtgcaagaggccccccccacgccatgatgaagagaggctacgccatggattactgggggcaaggcactctagtgaccgtgagtagtggcgggagcggtgggagcggcggttccgaaatcgtgctgacacagagccctggcactctgtctctgagtcccggcgagagagctaccctgagctgcagagcatctcagagcgtgggcagcagctacctggcctggtatcagcaaaagcccggccaagcccctagacttctgatctacggcgcattcagcagagccaccggcatccccgacagattcagtggatctggcagcggaacggacttcaccctaacaatcagccgtctggaacccgaagactttgcggtgtattactgtcaacagtacggtagcagcccctggaccttcggccaaggcacaaaggtggagatcaaaggcggttccggtggcgacaaaacacacacctgtcccccctgccccgcccctgaggctgccggtggtcctagcgtgttcctgttcccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgggcgcccccatcgagaagaccatcagcaaggccaaggggcagcctagagagccccaagtgtacaccctgccccctagcagagacgagctgaccaagaaccaagtgtcgctaacctgtttggtgaagggcttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgtcgctgagccccggaaaa

SEQ ID NO: 60 - protein, 1081 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHHHHH

SEQ ID NO: 61 - DNA, 3243 bp, synthetic construct
  source 1..3243: mol_type = other DNA
caagtgcaattggtagagtccggcggcggcgttgtgcaacccggcagaagccttagactgagctgcgccgcaagcggcttcaccttcagcagctacaccatgcactgggtgagacaagcccccggcaagggcctggaatgggtgaccttcatcagctacgacggcaacaacaagtactacgctgatagcgtgaagggcagattcactatcagccgtgacaacagcaagaacaccctgtacctgcagatgaacagcctgagagccgaggacaccgccatctactactgcgcaagaaccggctggctgggccccttcgactactggggccaagggacattagttactgttagcagcggcgggagcgggggcagcggaggatcggatatacagatgacacagagccctagcagcctgagcgcaagcgtgggcgacagagtgaccatcacctgcagcgccacaagcagcatcacctacatgagctggtatcagcaaaaacccggcaaggcccccaagctgctgatctacgacacaagcaacctggcaagcggcgtgcctagcagattctctggcagcggcagcggcacagattatactctcacaatcagcagtctgcagcctgaggatttcgccacatactactgccaacagtggagcagctaccccctgaccttcggcggcggcactaaagtagagattaaaggcggcagcggtggaagtggcgggagccaagtgcaactgcaagagagcggtcccggcctggtgaagccttctcagaccttgagccttacctgcaccgtgagcggcttcagcctgacaagctacggcgtgtactgggtgcggcaaccccccggcaaaggcttggaatggctgggggtgatctgggccggcggcaccaccaactacaacagcgccctgatgagcagactgaccattagcaaggacacaagcaagaaccaagtgagcctgaagctatctagcgtgaccgccgccgacaccgccgtctattactgtgcaagaggccccccccacgccatgatgaagagaggctacgccatggattattggggccaaggcaccctggttaccgtctcttccgggggtagcggcggcagcggaggttccgagatcgtgttaactcagagccctggcaccctgtcgctgagccctggtgaaagggctacgctaagctgcagagcatctcagagcgtgggcagcagctacctggcctggtatcagcagaaacccggccaagcccctagactgctgatctatggtgccttcagcagagccaccggcatccccgacagattcagcggttccggtagtggcacagacttcactctgaccatcagccggctagaacctgaggattttgccgtttactactgtcagcagtatggcagcagcccctggaccttcggccaaggcacgaaggtggagataaaaggtggaagcggcggggaagcccacaagagcgagatcgcccacagatacaacgacctgggcgagcagcacttcaagggcctggtgctgatcgccttctctcagtacctgcagaagtgcagctacgacgagcacgccaagctggtgcaagaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaagagccggagagaaatgagtgcttcctgcagcacaaggacgacaaccctagcctgccccccttcgagagacctgaggccgaggccatgtgcactagcttcaaggagaaccccaccaccttcatgggccactacctgcacgaggtggctagacgtcatccatatttctacgcccctgagctgctgtactatgcagagcagtacaacgagatcctgacccaatgttgtgccgaggcggacaaagagagctgcctgacccccaagctggacggcgtgaaggagaaggccctggtgagcagcgtcagacagagaatgaagtgcagcagcatgcagaagttcggggagagagctttcaaggcctgggccgtggctagactgtctcagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaagccactatctcatccaaattacagacctgctgcgacaagcccctgctgaagaaagctcattgcctcagcgaggtggagcatgacaccatgcccgccgatctgcccgccatcgccgccgacttcgtggaggaccaagaggtgtgcaagaactacgccgaggcgaaggatgtgttcctgggcaccttcctgtacgagtacagccgaaggcaccccgactacagcgtgagcctgctgctgagactggccaagaagtacgaggccactttggagaaatgctgcgctgaggcaaacccccctgcctgctacggcaccgtgctggccgagtttcagcccctggtagaggagccgaagaacctggtgaagaccaactgcgacctgtacgagaagctgggcgagtacggctttcagaacgccatcctggtgagatacacacagaaggccccccaagtgagcacccccaccttggtggaagccgcaagaaacctgggcagagtgggcaccaaatgttgcacgctgccggaggaccaaagactgccctgcgtggaggactacctgagcgccatcctgaacagagtgtgcctgctgcacgagaagacccccgtgagcgagcacgtgaccaagtgctgcagtggcagcctggtggagagaagaccctgcttcagcgccctgaccgtggacgagacctacgtgcccaaggagttcaaggccgagaccttcaccttccacagcgacatctgtactctccctgagaaggagaagcagatcaagaagcagaccgccctggccgagctggtgaagcacaagcccaaggccaccgccgagcagctgaagaccgtgatggacgacttcgctcagttcctggacacctgctgcaaggccgccgacaaggacacctgcttcagcaccgagggccccaacctggtgacaagatgcaaggacgccctggcccaccaccaccaccaccaccaccaccaccac

SEQ ID NO: 62 - protein, 449 aa, synthetic construct
DVQLQESGPGLVKPSQSLSLTCSVTGYSITSGYVWNWIRQFPGNKLEWMGYISHDGNTNYNPSLKNRISITRDTSKNQFFLKLNSVTTEDSATYYCTRNYGYGGTMDYWGQGTAVSVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 63 - DNA, 1347 bp, synthetic construct
  source 1..1347: mol_type = other DNA
gacgtgcagctgcaagagagcggccccggcctggtgaagccgagccaatctctgagcctgacctgcagtgtgacgggatactcaatcacctctggttacgtgtggaactggatcagacagttccccggcaacaagctggagtggatgggctacatcagccacgacggcaacaccaactacaaccctagcctgaagaacagaatcagcataacaagagacacaagcaagaatcagttcttcctgaagctgaacagcgtgaccaccgaggacagcgccacctactactgcacaagaaactacggctacggcggcaccatggactactggggccaaggcaccgccgtgagcgtaagcagcgccaagacaacagcccctagcgtgtacccgctggcccccgtttgcggcgacaccaccggtagcagcgtgaccctggggtgcctggtgaagggctacttccctgagcccgtgaccctgacttggaatagcggcagcctgagcagcggcgtgcacaccttccccgccgtgctgcagagcgacctgtacaccctgagcagcagcgtgacagtgacgagcagcacctggcctagccaaagtatcacctgcaacgtggcccaccccgcaagcagcaccaaggtggacaagaagatcgaaccaagaggccccactattaaaccttgccccccctgcaagtgtcccgcacccaatgcagcaggcggccctagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgctcagacacagacccacagagaggactacaacagcaccctgagagtggtgagcgccctgcccattcagcaccaagactggatgagcggcaaggagttcaagtgcaaggtgaacaacaaggacctgggcgcccccatcgagagaaccatcagcaagcccaagggcagcgtgagagccccccaagtgtacgtgctgcccccccctgaggaggagatgaccaagaagcaagtgaccctaacctgtatggtgaccgacttcatgcctgaggacatctacgtggagtggaccaacaacggcaagaccgagctgaactacaagaacaccgagcccgtgctggacagcgacggcagctacttcatgtacagcaagctgagagtggagaagaagaactgggtggagagaaacagctacagctgcagcgtggtgcacgagggcctgcacaaccaccacaccaccaagagcttcagcagaacccccggcaag

SEQ ID NO: 64 - protein, 214 aa, Mus sp.
DIVMTQSQKFMSTSVGDRVSVTCKASQNVGTYVAWYEQKLGQSPKALIFSASYRYTGVPDRFTGSGSGTDFTLTISNVQSEDLAEYFCQQYDSYPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - DNA, 642 bp, synthetic construct
  source 1..642: mol_type = other DNA
gacatcgtgatgacacagtctcagaagttcatgagcacaagcgtgggcgacagagtgagcgtgacctgcaaggcatctcagaacgtgggcacctacgtggcctggtacgagcagaagctggggcagagccccaaggccctgatcttcagcgcaagctacagatataccggcgtgcccgacagattcaccggcagcggcagcggcaccgacttcaccctgaccatcagcaacgtgcagagcgaggacctggccgagtacttctgtcagcagtacgacagctaccccctgaccttcggcggcggcaccaagctcgagatcaagcgcgcagatgctgctcctaccgtgagcatcttcccgccgtccagcgaacaactcactagcggaggcgcgtcagtggtctgcttccttaacaatttctaccctaaggacatcaacgtcaagtggaagattgacggatcggaacgccagaacggagtgctgaactcatggactgatcaggattccaaagactcgacttactccatgtccagcaccctgaccctgaccaaagacgagtacgaaaggcacaactcgtacacgtgcgaagccacccacaagacttccacctcgcccatcgtgaagtccttcaatcgcaatgagtgc

SEQ ID NO: 66 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1344 bp, synthetic construct
  source 1..1344: mol_type = other DNA
caagtgcagctagttgagtctggaggcggcgtggtgcagcccggcagaagcctgagactgagctgcgccgcaagcggcttcaccttcagcagctacaccatgcactgggtgagacaagcccccggcaagggcctggagtgggtgaccttcatcagctacgacggcaacaacaagtactacgccgacagcgtgaagggcagattcaccatcagcagagacaacagcaagaacaccctgtacctgcagatgaacagcctgagagccgaggacaccgccatctactactgcgctagaaccggctggctgggccccttcgactactggggccaaggcaccctggtaacggtaagcagcgctagtaccaaagggcctagcgtgtttcctttagcccctagcagcaaaagcacaagcggaggcaccgccgccctgggctgtctggtgaaggattactttcctgaacccgtgactgtatcatggaatagcggggccctgactagcggagtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaagagctgtgacaaaacgcacacctgtccgccgtgccccgcccctgaggctgccggcggccctagcgtgttcctgtttccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgggcgcccccatcgagaagaccatcagcaaggccaaggggcagcctagagagccccaagtgtacaccctgccccctagcagagacgagctgaccaagaaccaagtgagcctgacctgcctagtgaagggtttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgagcctgagccccggcaag

SEQ ID NO: 68 - protein, 215 aa, synthetic construct; identical to SEQ ID NO: 28

SEQ ID NO: 69 - DNA, 651 bp, synthetic construct
  source 1..651: mol_type = other DNA
gagatcgtgctgacacagagccccggcacactgtctctgagccccggcgagagagccacactgagctgcagagccagccagagcgtgggcagcagctatctggcttggtaccagcagaagcccggccaagcccctagactgctgatttacggagcctttagcagagccaccggcatccccgacagattcagcggaagcggcagcggcaccgatttcacactgacaatctctagactggagccagaggacttcgccgtgtactactgccagcagtacggcagcagcccttggacctttggccaaggcaccaaggtggagatcaagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 70 - protein, 119 aa, synthetic construct
DVQLQESGPGLVKPSQSLSLTCSVTGYSITSGYVWNWIRQFPGNKLEWMGYISHDGNTNYNPSLKNRISITRDTSKNQFFLKLNSVTTEDSATYYCTRNYGYGGTMDYWGQGTAVSVSS

SEQ ID NO: 71 - protein, 107 aa, synthetic construct
DIVMTQSQKFMSTSVGDRVSVTCKASQNVGTYVAWYEQKLGQSPKALIFSASYRYTGVPDRFTGSGSGTDFTLTISNVQSEDLAEYFCQQYDSYPLTFGGGTKLEIK

SEQ ID NO: 72 - protein, 119 aa, synthetic construct; identical to SEQ ID NO: 70

SEQ ID NO: 73 - protein, 107 aa, synthetic construct; identical to SEQ ID NO: 71

SEQ ID NO: 74 - protein, 119 aa, synthetic construct
DVQLQESGPGLVKPSQTLSLTCTVTGYSITSGYVWNWIRQPPGKGLEWMGYISHDGNTNYNPSLKSRVTISRDTSKNQFSLKLSSVTAADTAVYYCTRNYGYGGTMDYWGQGTTVTVSS

SEQ ID NO: 75 - protein, 107 aa, synthetic construct
DIQMTQSPSSLSASVGDRVTITCRASQNVGTYVAWFQQKPGKAPKALIFSASYRYTGVPSRFSGSGSGTDFTLTISSLQPEDFATYFCQQYDSYPLTFGGGTKLEIK

SEQ ID NO: 76 - protein, 119 aa, synthetic construct; identical to SEQ ID NO: 74

SEQ ID NO: 77 - protein, 107 aa, synthetic construct
DIQMTQSPSSLSASVGDRVTITCRASQNVGTYVAWFQQKPGKAPKALIFSASYLYSGVPSRFSGSGSGTDFTLTISSLQPEDFATYYCQQYDSYPLTFGGGTKLEIK

SEQ ID NO: 78 - protein, 119 aa, synthetic construct; identical to SEQ ID NO: 74

SEQ ID NO: 79 - protein, 107 aa, synthetic construct
DIVMTQSPDSLAVSLGERATINCKASQNVGTYVAWYQQKPGQPPKALIFSASYRYTGVPDRFSGSGSGTDFTLTISSLQAEDVAVYFCQQYDSYPLTFGGGTKLEIK

SEQ ID NO: 80 - protein, 119 aa, synthetic construct; identical to SEQ ID NO: 74

SEQ ID NO: 81 - protein, 107 aa, synthetic construct
DIVMTQSPDSLAVSLGERATINCKASQNVGTYVAWYQQKPGQPPKALIFSASYRYSGVPDRFSGSGSGTDFTLTISSLQAEDVAVYYCQQYDSYPLTFGGGTKLEIK

SEQ ID NO: 82 - protein, 119 aa, synthetic construct
QVQLQESGPGLVKPSQTLSLTCTVTGYSITSGYVWSWIRQPPGKGLEWIGYISHDGNTNYNPSLKSRVTISRDTSKNQFSLKLSSVTAADTAVYYCTRNYGYGGTMDYWGQGTTVTVSS

SEQ ID NO: 83 - protein, 107 aa, synthetic construct; identical to SEQ ID NO: 75

SEQ ID NO: 84 - protein, 119 aa, synthetic construct; identical to SEQ ID NO: 82

SEQ ID NO: 85 - protein, 107 aa, synthetic construct; identical to SEQ ID NO: 77

SEQ ID NO: 86 - protein, 119 aa, synthetic construct; identical to SEQ ID NO: 82

SEQ ID NO: 87 - protein, 107 aa, synthetic construct; identical to SEQ ID NO: 79

SEQ ID NO: 88 - protein, 119 aa, synthetic construct; identical to SEQ ID NO: 82

SEQ ID NO: 89 - protein, 107 aa, synthetic construct; identical to SEQ ID NO: 81

SEQ ID NO: 90 - protein, 119 aa, synthetic construct
DVQLKESGPALVKPTQTLTLTCTVTGYSITSGYVWNWIRQPPGKALEWMGYISHDGNTNYNPSLKTRLTISRDTSKNQFVLTMTNMDPVDTATYYCTRNYGYGGTMDYWGQGTTVTVSS

SEQ ID NO: 91 - protein, 107 aa, synthetic construct; identical to SEQ ID NO: 75

SEQ ID NO: 92 - protein, 119 aa, synthetic construct; identical to SEQ ID NO: 90

SEQ ID NO: 93 - protein, 107 aa, synthetic construct; identical to SEQ ID NO: 77

SEQ ID NO: 94 - protein, 119 aa, synthetic construct; identical to SEQ ID NO: 90

SEQ ID NO: 95 - protein, 107 aa, synthetic construct; identical to SEQ ID NO: 79

SEQ ID NO: 96 - protein, 119 aa, synthetic construct; identical to SEQ ID NO: 90

SEQ ID NO: 97 - protein, 107 aa, synthetic construct; identical to SEQ ID NO: 81

SEQ ID NO: 98 - protein, 119 aa, synthetic construct
QVTLKESGPALVKPTQTLTLTCTVTGYSITSGYVVSWIRQPPGKALEWIGYISHDGNTNYNPSLKTRLTISRDTSKNQFVLTMTNMDPVDTATYYCTRNYGYGGTMDYWGQGTTVTVSS

SEQ ID NO: 99 - protein, 107 aa, synthetic construct; identical to SEQ ID NO: 75

SEQ ID NO: 100 - protein, 119 aa, synthetic construct; identical to SEQ ID NO: 98

SEQ ID NO: 101 - protein, 107 aa, synthetic construct; identical to SEQ ID NO: 77

SEQ ID NO: 102 - protein, 119 aa, synthetic construct; identical to SEQ ID NO: 98

SEQ ID NO: 103 - protein, 107 aa, synthetic construct; identical to SEQ ID NO: 79

SEQ ID NO: 104 - protein, 119 aa, synthetic construct; identical to SEQ ID NO: 98

SEQ ID NO: 105 - protein, 107 aa, synthetic construct; identical to SEQ ID NO: 81

SEQ ID NO: 106 - protein, 16 aa, synthetic construct
QRFCTGHFGGLYPCNG

SEQ ID NO: 107 - protein, 16 aa, synthetic construct
QRFCTGHFGGLHPCNG

SEQ ID NO: 108 - protein, 16 aa, synthetic construct
QRFVTGHFGGLYPANG

SEQ ID NO: 109 - protein, 16 aa, synthetic construct
QRFVTGHFGGLHPANG

SEQ ID NO: 110 - protein, 4 aa, synthetic construct
YVKM

SEQ ID NO: 111 - protein, 8 aa, synthetic construct
EPKSCDKT

SEQ ID NO: 112 - protein, 34 aa, synthetic construct
GGCGGGEVAALEKEVAALEKEVAALEKEVAALEK

SEQ ID NO: 113 - protein, 34 aa, synthetic construct
GGCGGGKVAALKEKVAALKEKVAALKEKVAALKE

SEQ ID NO: 114 - protein, 6 aa, synthetic construct
HHHHHH

SEQ ID NO: 115 - protein, 449 aa, Mus sp.
DVQLQESGPGLVKPSQSLSLTCSVTGYSITSGYVWNWIRQFPGNKLEWMGYISHDGNTNYNPSLKNRISITRDTSKNQFFLKLNSVTTEDSATYYCTRNYGYGGTMDYWGQGTAVSVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 116 - protein, 9 aa, synthetic construct
GGSGGSGGS

SEQ ID NO: 117 - protein, 492 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKHHHHHHHHHH

SEQ ID NO: 118 - protein, 8 aa, synthetic construct
GGGSGGGG

SEQ ID NO: 119 - protein, 15 aa, synthetic construct
GGGGSGGGGSGGGGS

SEQ ID NO: 120 - protein, 18 aa, synthetic construct
KEAKEAKEAKEAKEAKEA

(19) United States
(12) Patent Application Publication
(10) Pub. No.: US 2026/0053913 A1
(43) Pub. Date: Feb. 26, 2026

(54) COMBINATIONS OF CTLA4 BINDING PROTEINS AND METHODS OF TREATING CANCER

(71) Applicant: BioEntre LLC, Brookline, MA (US)

(72) Inventors: Gordon Gokleun Wong, Brookline, MA (US); Vincent Ling, Walpole, MA (US)

(21) Appl. No.: 18/873,448

(22) PCT Filed: Jun. 12, 2023
(86) PCT No.: PCT/US2023/068282

§ 371 Date: Dec. 10, 2024

Related U.S. Application Data
(60) Provisional application No. 63/351,005, filed on Jun. 10, 2022.

(51) Int. Cl. A61K 39/395; A61K 39/00; A61P 37/04; C07K 16/28

(52) U.S. Cl. CPC A61K 39/39541; A61P 37/04; C07K 16/2818; A61K 2039/507; C07K 2317/24; C07K 2317/52; C07K 2317/76; C07K 2317/94; C07K 2319/31

(57) ABSTRACT

The present disclosure provides antigen-binding proteins that bind to cytotoxic T-lymphocyte-associated antigen-4 (CTLA4) as well as combinations of certain antigen-binding proteins that are particularly effective in modulating the CTLA4 signaling pathway. Nucleic acids, vectors, host cells, and conjugates are also provided herein. Further provided are kits and pharmaceutical compositions comprising said entities as well as methods of making said antigen-binding proteins and methods of treatment.

### CROSS-REFERENCE TO RELATED APPLICATIONS

This application claims the benefit of U.S. Provisional Application No. 63/351,005, filed on Jun. 10, 2022, the entire contents of which is incorporated herein in its entirety by this reference.

### BACKGROUND

The striking clinical success of cancer immunotherapy with checkpoint blockade suggests it is likely to form the foundation of curative therapy for many malignancies (Reck et al. (2016) N. Engl. J. Med. 375:1823-1833; Hodi et al. (2010) N. Engl. J. Med. 363:711-723). The cytotoxic T-lymphocyte-associated antigen-4 (CTLA4) is an immune checkpoint that regulates T-cell proliferation at the early stage of naive T-cell activation, principally in the lymph nodes, thus providing a negative signal to T cells. Blockade of CTLA4 binding to its cognate ligand(s) induces an antitumor immune response by promoting the activation and proliferation of tumor-specific T cells.

Ipilimumab (Yervoy), a human monoclonal antibody that binds to human CTLA4 and blocks its interaction with ligands, demonstrated clinical efficacy in patients with melanoma, renal cell carcinoma, prostate cancer, urothelial carcinoma, and ovarian cancer. In 2011, ipilimumab was approved by the U.S. Food and Drug Administration (FDA) for the treatment of melanoma, and its 2020 sale is estimated at $1.7 billion. However, ipilimumab has only a 22% long-term success rate in melanoma eradication, albeit effectively a cure. Additionally, ipilimumab is highly toxic with many side effects characteristic of autoimmune disease and TREG (Regulatory T cells) depletion, thereby limiting its broad use. Accordingly, a great need exists for additional immunotherapeutic strategies with better efficacy and safety profiles.

### SUMMARY

The present invention is based, at least in part, on the discovery that certain CTLA4-binding proteins, or combinations thereof, show unexpected T cell activation properties.

In certain aspects, provided herein are humanized CTLA4-binding proteins that are surprisingly effective in blocking the CTLA4 signaling pathway. For example, these CTLA4-binding proteins outperformed the clinically validated Ipilimumab in assays evaluated herein.

In certain aspects, also provided herein are combinations of CTLA4-binding proteins that show synergistic effects in blocking the CTLA4 signaling pathway.

The antigen-binding proteins or any combinations thereof presented herein may be recombinant or engineered.

The antigen-binding proteins or any combinations thereof presented herein may or may not bind one or more Fc receptors. In some embodiments, the antigen-binding protein comprises a fully functional Fc domain that binds to one or more Fc receptors. In other embodiments, the antigen-binding protein lacks the Fc domain, or comprises an Fc domain comprising a certain mutation (e.g., LALA mutation, LALAPG mutation) that renders them ineffective in binding to one or more Fc receptors.

In certain aspects, provided herein is an isolated nucleic acid molecule that encodes an antigen-binding protein of the present disclosure. Also provided herein is a vector comprising such nucleic acid molecule. Further provided herein is a host cell which comprises the isolated nucleic acid, comprises a vector, or expresses an antigen-binding protein of the present disclosure.

In certain aspects, provided herein is a pharmaceutical composition of an antigen-binding protein of the present disclosure, a combination of at least two antigen-binding proteins, the isolated nucleic acid, the vector, or the host cell. Further provided herein is a kit comprising at least one antigen-binding protein of the present disclosure.

In certain aspects, provided herein is a method of producing an antigen-binding protein of the present disclosure, wherein the method comprises the steps of: (i) culturing a host cell comprising a nucleic acid comprising a sequence encoding the antigen-binding protein of the present disclosure under conditions suitable to allow expression of said antigen-binding protein; and (ii) recovering the expressed antigen-binding protein.

In certain aspects, provided herein is a method of preventing or treating a subject afflicted with a cancer or other CTLA4-related disease(s), the method comprising administering to the subject an antigen-binding protein of the present disclosure, a combination of at least two antigen-binding proteins, or a pharmaceutical composition comprising same.

In certain aspects, provided herein is a method of reducing proliferation of a cancer cell in a subject, the method comprising administering to the subject an antigen-binding protein of the present disclosure, a combination of at least two antigen-binding proteins, or a pharmaceutical composition comprising same.

### BRIEF DESCRIPTION OF THE DRAWINGS

FIG. 1 shows the CTLA4 blockade assay. The bioassay consists of two genetically engineered cell lines, CTLA4 Effector Cells (Jurkat) and aAPC/Raji Cells. When co-cultured, the CTLA4/CD80 and CD86 interaction inhibits the CD28 pathway activated luminescence (left panel). The addition of anti-CTLA4 antibody blocks the CTLA4/CD80 and CD86 interaction, thereby re-establishing the CD28 pathway activated luminescence, which can be detected in a dose-dependent manner by addition of a luminescent agent (Glo) and quantitation with a luminometer (middle panel). The functional readout for this cell assay is the expression of (i) endogenous IL-2 and/or (ii) a luciferase under the IL-2 promoter.

FIG. 2A shows the CTLA4 blockade dose response to ipilimumab (“Ipi” or “IPI”), L3D10, or a combination of IPI and L3D10. RLU was the readout.

FIG. 2B shows the CTLA4 blockade dose response to ipilimumab (“Ipi” or “IPI”) or L3D10 with the IL-2 level as an assay output. RLU Readout was equivalent to IL-2 Readout. Blockade activity for IPI=L3D10=JA1020. IPI at 4000 nM and IPI at 1000 nM showed same activity titration curve.

FIG. 3A shows a graph of titration curve αCTLA 4 antibody Ipilimumab and αPD1 antibody Nivolumab as single agents and in combination (adapted from Promega's assay manual). Assay response to anti-PD1, anti-CTLA4 or a mixture of anti-PD1 plus anti-CTLA4 antibodies using PD1+CTLA4 combination bioassay. The PD1+CTLA4 effector cells, thaw-and-use were incubated with PD-L1 aAPC/Raji cells, Thaw-and-use the cells and a serial titration of anti-PD1 nivolumab, anti-CTLA4 ipilimumab or a mixture of nivolumab plus ipilimumab antibodies. After 6 hours induction at 37° C., Bio-Glo™ reagent was added and luminescence determined using a GloMax® Discover luminometer. Four-parameter logistic curve analysis was performed with GraphPad Prism® software.

Note the marginal response of the single agents plateauing at about 3× stimulation above background while when used in combination the luciferase response is 20× above background and 6× either agent alone.

FIG. 3B shows the CTLA4 and PD-1 checkpoint pathways, and proposed mechanisms of action of anti-CTLA4 and anti-PD-1 antibodies. (Adapted from Wilsmore et al. (2021)). (A) The CTLA4 and PD-1 pathways negatively regulate Tcell activation. Tcell receptor (TCR) engagement with an antigen presented via the major histocompatibility complex (MHC) requires a costimulatory second signal for activation delivered via CD28. CTLA4 is a competitive CD28 homolog that binds CD28 ligands CD80/86, preventing Tcell activation. CTLA4 also mediates transendocytosis of CD28 ligands CD80/86. PD-1 engages with its ligand PD-L1 to negatively regulate Tcell activation. CD28 is also a secondary target for PD-1 and a point of convergence of the two pathways. Both CTLA4 and PD-1 expression are upregulated upon TCR activation. Intracellular signaling for both pathways is mediated via the phosphatase Src homology region-2 containing protein tyrosine phosphatase (SHP-2) inhibiting PI3K downstream signaling. CTLA4 in addition interacts with the serine/threonine phosphatase PP2A which dephosphorylates AKT, further inhibiting the pathway. (B) (1) Anti-CTLA4 restores Tcell activation by inhibiting interaction between CTLA4 and CD80/CD86 on APC. (2) Anti-CTLA4 may inhibit transendocytosis of CD28 ligands CD80/86 mediated through CTLA4. (3) The anti-CTLA4 IgG1 antibody Ipilimumab can engage FcγRs on immune effector cells (NK cells, monocytes/macrophages) via its Fc region, leading to antibody-dependent cellular cytotoxicity (ADCC) and depletion of some high-CTLA4-expressing Tcell subsets (e.g. Tregs). (4) Anti-PD-1 restores Tcell activation by inhibiting the interaction between PD-1 on T cells and PD-L1 (PD-L1 may be expressed by tumor cells and various immune cells). (5) Anti-PD-1 restores Tcell activation by interaction between PD-1 and CD28 point of convergence of the two pathways. ADCC, antibody-dependent cellular cytotoxicity; APC, antigen-presenting cell; CTLA4, cytotoxic T lymphocyte antigen-4; FcγRs, Fc gamma receptors; TCR, Tcell receptor; MHC, major histocompatibility complex; NK cells, Natural Killer cells; PD-1, programmed death-1; PD-L1, programmed death-1 ligand; PI3K/Akt, phosphatidylinositol 3-kinase (PI3K) and Akt/Protein Kinase B; SHP-2, phosphatase Src homology region-2 containing protein tyrosine phosphatase; Treg, regulatory T cells.

FIG. 4A-FIG. 4B show the αCTLA4/PD1 functional blockade assays (RLU readout) of two incubation times. FIG. 4A shows the results after 6 hours, and FIG. 4B shows the results after 20 hours.

FIG. 5A-FIG. 5B show the CytoStim™/LPS primary human T cell activation assay using human PBMC from two donors: Donor A (FIG. 5A) and Donor B (FIG. 5B). IL-2 induction was determined after 48 hours of incubation.

FIG. 6A-FIG. 6B show the αCTLA4 functional blockade assay. FIG. 6A and FIG. 6B show the surprising and unexpected heightened activity of BioE2052 as compared to Ipilimumab (IPI), BioE2201 (IPI Fab), Bio2202 (121 Fab), and BioE2201+BioE2202.

FIG. 7 shows surprising synergism between BNI3 and full-length IPI, as well as between BNI3 and full-length 121 in the αCTLA4 functional blockade assay.

FIG. 8 shows surprising synergism between BNI3 and IPI Fab2, as well as between BNI3 and 121 Fab2 in the αCTLA4 functional blockade assay.

FIGS. 9A and 9B show anti-CTLA4 functional blockade combinatorial analysis.

Entries are the ratios of test agent RLU/No test agent RLU in Promega anti-CTLA4 functional blockade assay.

FIG. 10 shows anti-CTLA4 functional blockade assay of BNI3 and IPI combinations.

FIG. 11 shows anti-CTLA4 functional blockade assay of BNI3 and BioE2032 combinations.

FIG. 12A-FIG. 12B show anti-CTLA4 functional blockade assay with or without Raji cells.

FIG. 13A-FIG. 13B show anti-CTLA4/anti-PD1 functional blockade assay (6 hours of incubation).

FIG. 14A-FIG. 14B show anti-CTLA4/anti-PD1 functional blockade assay (20 hours of incubation).

FIG. 15A-FIG. 15B show anti-CTLA4/anti-PD1 functional blockade assay (22 hours of incubation).

FIG. 16 shows CytoStim™+LPS stimulated Hu PBMC (Donor A, STD37-5A), anti-CTLA4 and anti-PD1 agents with IL-2 Readout. FIG. 16 shows BioE2052+/−Nivolumab vs Ipilimumab+/−Nivolumab in affecting IL-2 response in CytoStim™+LPS activated Human PBMC by methodology described in Dovedi et al. ‘21. nivo=Nivolumab, IPI=Ipilumamb, BioE2033=121 Fab2, BioE2052.

Three human PBMC donors were stimulated with Cytostim™ (Milteni Biotec, 1 in 400 dilution) and LPS (100 ng/mL) in the presence of the test agents. Supernatant was harvested after 48h and 72h incubation for IL-2 assessment (ELISA).

Ipilimumab and human IgG1 isotype control were assessed in parallel and the following controls were included for each donor: (i) PBMC Only, (ii) PBMC+Cytostim, (iii) PBMC+LPS, (iv) PBMC+Cytostim+LPS (No sample).

FIG. 17A-FIG. 17B show anti-CTLA4 functional blockade assay of the humanized BNI3 variants.

FIG. 18A-FIG. 18B show anti-CTLA4 functional blockade assay of the humanized BNI3 variants in combination with IPI or BioE2001.

FIG. 19 shows anti-CTLA4 functional blockade assay of BioE2551.

FIG. 20 shows anti-CTLA4 functional blockade assay of BioE2450, BioE2032, and BioE2460.

FIG. 21 shows anti-CTLA4 functional blockade assay of hBNI3-v2+BioE2032, hBNI3-v2+BioE2460, BioE2551+BioE2032, and BioE2551+BioE2460.

FIG. 22 shows anti-CTLA4 functional blockade assay of hBNI3-v2+IPI, hBNI3-v2 +BioE2450, BioE2551+IPI, BioE2551+BioE2450.

FIG. 23 shows the summary of anti-CTLA4 functional blockade activity. Ratio of the activity of engineered anti-CTLA4 binding proteins to that of IPI is shown.

FIG. 24 shows anti-CTLA4 functional blockade assay of BioET1100, BioET1300, and BioET1500 (Ratio of RLU).

FIG. 25 shows anti-CTLA4/anti-PD1 functional blockade assay. The relative luciferase units (RLU) readout is shown for IPI, BioET1100, BioET1300, and BioET1500.

FIG. 26 shows an exemplary in vivo treatment design for MC38 mouse tumor model.

FIG. 27A-FIG. 27B show exemplary anti-CTLA4-binding proteins. FIG. 30A shows the domain structure of exemplary anti-CTLA4 diabodies (BioE2051 and BioE2052). The diabodies comprise the VL and VH domains of ipilimumab and 121 antibody, and target two independent epitopes on CTLA4. FIG. 30B shows the diagram and sequence of BioE2052.

FIG. 28 summarizes the results of a CTLA4 Functional Blockade assay of BioE2420.

### DETAILED DESCRIPTION

Provided herein are certain CTLA4-binding proteins, or combinations thereof, that show unexpected T cell activation properties.

### Definitions

The articles “a” and “an” are used herein to refer to one or to more than one (i.e. to at least one) of the grammatical object of the article. By way of example, “an element” means one element or more than one element.

The term “antigen presenting cell” includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes).

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions.

The term “conjoint” or “conjoint therapy,” with respect to administration of two or more agents, refers to the simultaneous, sequential or separate dosing of the individual agents provided that some overlap occurs in the simultaneous presence of the agents or compositions in a cell or a subject. The different agents comprising the conjoint therapy may be administered concomitant with, prior to, or following the administration of one or more therapeutic agents, such that some overlap occurs in the simultaneous presence of the agents in a cell or a subject. As used herein, administration of any composition or pharmaceutical composition comprising two or more agents encompasses a conjoint administration of two or more agents.

By “detectable label” is meant a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

As used herein, the term “Fc region” or “Fc domain” is used to describe a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present invention include human IgG1, IgG2 (IgG2A, IgG2B), IgG3 and IgG4.

As used herein, “Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see M. Daëron, Annu. Rev. Immunol. 15:203-234 (1997). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

As used herein, the term “FcRnBP” refers to an engineered FcRN-binding peptides, which when fused to a protein, it extends the half-life of the protein in plasma. Exemplary peptides are described in Datta-Mannan et al. (2018) Biotechnology Journal, 14 (3): e1800007; Mezo et al. (2008) Proc Natl Acad Sci U.S.A., 105 (7): 2337-2342; Sockolosky et al. (2012) Proc Natl Acad Sci U.S.A., 109 (40): 16095-16100; each of which is incorporated by reference. IN some embodiments, such peptides include those having an aminio acid sequence of QRFCTGHFGGLYPCNG; QRFCTGHFGGLHPCNG; QRFVTGHFGGLYPANG; or QRFVTGHFGGLHPANG. The FcRnBP can be linear or cyclical.

Antibodies may be “humanized” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

“Interleukin-2 (IL-2)” is a cytokine signaling molecule that functions in the immune system. The IL-2 protein is produced primarily by activated T cells (CD4+ T cells); it regulates the activities of other T cells and B cells (increases growth and activity of these white blood cells) that are responsible for immunity. IL-2 is classified as a biologic response modifier that can modify the body's response to cancer cells. The production of IL-2 exerts a wide spectrum of immunoregulatory effects on the immune system, e.g., increasing the proliferation and/or functional activity of other immune cells, such as tumor-infiltrating lymphocytes (TILs; T cells) and natural killer (NK) cells, enhancement of lymphocyte mitogenesis, lymphocyte cytotoxicity, induction of NK cells and lymphokine activated NK cells, and induction of interferon-γ production (S. L. Gaffen et al., 2004, Cytokine, 28 (3): 109-23). In T cells, IL-2 synthesis is tightly regulated at the mRNA level by signals from the T cell receptor (TCR) and CD28.

As used herein, the term “KD” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “remission” is art recognized, and refers to a condition in which the signs and symptoms of the cancer are reduced.

The term “selective” refers to a preferential action or function. The term “selective” can be quantified in terms of the preferential effect in a particular target of interest relative to other targets. For example, a measured variable (e.g., binding of the CTLA4-binding protein or the CTLA4-blocking activity) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, measured effect, and the like.

By contrast, the term “specific” refers to an exclusionary action or function. For example, specific binding of an antibody or antigen-binding protein to a predetermined antigen refers to the ability of the antibody or antigen-binding protein to bind to the antigen of interest without binding to other antigens. Typically, the antibody binds with an affinity (K_{D}) of approximately less than 1×10⁻⁷ M, such as approximately less than 10⁻⁸ M, 10⁻⁹ M, 10⁻¹⁰ M, 10⁻¹¹ M, or even lower to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen.

The term “sensitize” means to alter cells, such as cancer cells or tumor cells, in a way that allows for more effective treatment with a therapy (e.g., a CTLA4-binding protein). In some embodiments, normal cells are not affected to an extent that causes the normal cells to be unduly injured by the therapy (e.g., a CTLA4-binding protein). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42:2159-2164), cell death assays (Weisenthal L M, Shoemaker R H, Marsden J A, Dill P L, Baker J A, Moran E M, Cancer Res 1984; 94:161-173; Weisenthal L M, Lippman M E, Cancer Treat Rep 1985; 69:615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal L M, Contrib Gynecol Obstet 1994; 19:82-90). The sensitivity or resistance may also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a CTLA4-binding protein, can be equally applied to methods for sensitizing hyperproliferative or otherwise cancerous cells (e.g., resistant cells) to the therapy.

As used herein, “subject” refers to any healthy animal, mammal or human, or any animal, mammal or human afflicted with a cancer. The term “subject” is interchangeable with “patient.”

The term “synergistic effect” refers to the combined effect of two or more therapeutic agents, such as two or more CTLA4 pathway modulators, either alone or in combination with another cancer therapy can be greater than the sum of the separate effects of individual agents alone. As used herein, the synergistic effect may also be used to refer to the effect of a single CTLA4-binding protein that comprises two or more binding moieties, wherein the effect (e.g., biological effect or therapeutic effect) is greater than the sum of the separate effects of the individual binding moieties.

Conventional T cells, also known as Tcons or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+Teffs or CD8+Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naïve Tcons” are CD4″ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present disclosure which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD50 and the ED50. Compositions that exhibit large therapeutic indices are preferred. In some embodiments, the LD50 (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED50 (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC50 (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. Cancer cell death can be promoted by at least about 10%, 15%, 20%, 25%, 30%, 35%, 40%, 45%, 50%, 55%, 60%, 65%, 70%, 75%, 80%, 85%, 90%, 95%, or even 100%. In other embodiments, at least about a 10%, 15%, 20%, 25%, 30%, 35%, 40%, 45%, 50%, 55%, 60%, 65%, 70%, 75%, 80%, 85%, 90%, 95%, or even 100% decrease in cancer cell numbers and/or a solid malignancy can be achieved.

### CTLA4, Epitopes, and Ipilimumab

T cells, both CD4 (helper) and CD8 (cytotoxic), contribute to the adaptive immune response against pathogens and tumors, and activation and recruitment of specific T cells constitute a complex process. For a T cell to become fully activated (and subsequently proliferate and mediate effector function), at least 2 receptor-ligand interactions are required. The first of these occurs when the unique receptor of the T cell recognizes its cognate ligand, a short peptide presented in the context of a MHC molecule. This interaction is exquisitely specific, and if a good fit occurs, T-cell activation is initiated. However, full activation of a CD4 or CD8 T cell requires a second signal transmitted by costimulatory molecules present on the same antigen-presenting cell that expresses the peptide/MHC. This second signal is transmitted from costimulatory molecules (B7-1 (CD80) and/or B7-2 (CD86)) to a receptor on T cells known as CD28. Only when both signals are received and integrated does a specific T cell proliferate, acquire effector function, and migrate to sites of antigen expression.

CTLA4 is a homolog of CD28, suggesting that CTLA4 might serve, along with CD28, as a costimulatory molecule. However, several other studies provided opposing results, and for some time, it was not clear whether CTLA4 transmitted a stimulatory or inhibitory signal to T cells. The generation of mice lacking CTLA4 provided a solution for this conundrum: Knockout mice developed a progressive accumulation of activated T cells and died of lymphoproliferative disease ˜3 to 4 weeks after birth. These and other results suggested that blockade of CTLA4 with a monoclonal antibody could augment an adaptive immune response to an infectious agent or an evolving tumor. The subsequent study showed that CTLA4 blockade could attenuate the growth of several implanted murine tumors.

CTLA4 (also known as CD152) is a protein receptor that functions as an immune checkpoint and downregulates immune responses. CTLA4 is constitutively expressed in regulatory T cells but only upregulated in conventional T cells after activation—a phenomenon which is particularly notable in cancers. It acts as an “off” switch when bound to CD80 or CD86 on the surface of antigen-presenting cells. Thus, CTLA4 is a member of the immunoglobulin superfamily and encodes a protein which transmits an inhibitory signal to T cells.

The protein contains an extracellular V domain, a transmembrane domain, and a cytoplasmic tail. Alternate transcriptional splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. The first role of CTLA4 in inhibiting T cell responses seem to be directly via SHP-2 and PP2A dephosphorylation of TCR-proximal signaling proteins such as CD3 and LAT. CTLA4 can also affect signaling indirectly via competing with CD28 for CD80/86 binding. CTLA4 can also bind PI3K, although the importance and results of this interaction are uncertain. CTLA4 is known to interact with various proteins such as CD80, CD86, CTXN3, MALL, PIK3R1, and TMEM218.

Mutations in CTLA4 have been associated with insulin-dependent diabetes mellitus, Graves' disease, Hashimoto thyroiditis, celiac disease, systemic lupus erythematosus, thyroid-associated orbitopathy, and other autoimmune diseases.

The nucleic acids and polypeptide sequences of CTLA4 in humans and other organisms are well-known and include, for example, human CTLA4 (NM_001037631.3→NP_001032720.1 cytotoxic T-lymphocyte protein 4 isoform CTLA4del™; NM 005214.5 →NP_005205.2 cytotoxic T-lymphocyte protein 4 isoform CTLA4-TM precursor), mouse CTLA4 (NM_001281976.1→NP_001268905.1 cytotoxic T-lymphocyte protein 4 isoform 2 precursor; NM_009843.4→NP_033973.2 cytotoxic T-lymphocyte protein 4 isoform 1 precursor), and rat (NM_031674.1→NP_113862.1 cytotoxic T-lymphocyte protein 4 precursor). Representative nucleic acid and polypeptide sequences are disclosed in Table 1 below.

TABLE 1
Exemplary sequences of CTLA4 and its epitopes

SEQ ID NO: 1 Epitope 1 of CTLA4 (amino acid residues 134-139 of CTLA4)

| | |
|---|---|
| 1 | mypppy |

SEQ ID NO: 2 Epitope 2 of CTLA4 (amino acid residues 65-68 of CTLA4)

| | |
|---|---|
| 1 | sict |

*The amino acid residues of 65-68 of ⁶⁵SICT⁶⁸ (SEQ ID NO: 2) correspond to the
amino acid residues of the mature CTLA4 polypeptide. SEQ ID NO: 3 is the
sequence of the CTLA4 polypeptide before maturation, thus the SICT epitope (SEQ
ID NO: 2) corresponds to the amino acid residues 101-104 of SEQ ID NO: 3.
SEQ ID NO: 3 Human CTLA4 isoform CTLA4-TM amino acid sequence (NP_005205.2)

| | |
|---|---|
| 1 | maclgfqrhk aqlnlatrtw pctllffllf ipvfckamhv aqpavvlass rgiasfvcey |
| 61 | aspgkatevr vtvlrqadsq vtevcaatym mgneltfldd sictgtssgn qvnltiqglr |
| 121 | amdtglyick velmypppyy lgigngtqiy vidpepcpds dfllwilaav ssglffysfl |
| 181 | ltavslskml kkrsplttgv yvkmpptepe cekqfqpyfi pin |

SEQ ID NO: 4 Human CTLA4 transcript variant 1 cDNA sequence (NM_005214.5;
CDS 173-844)

| | |
|---|---|
| 1 | gctttctatt caagtgcctt ctgtgtgtgc acatgtgtaa tacatatctg ggatcaaagc |
| 61 | tatctatata aagtccttga ttctgtgtgg gttcaaacac atttcaaagc ttcaggatcc |
| 121 | tgaaaggttt tgctctactt cctgaagacc tgaacaccgc tcccataaag ccatggcttg |
| 181 | ccttggattt cagcggcaca aggctcagct gaacctggct accaggacct ggccctgcac |
| 241 | tctcctgttt tttcttctct tcatccctgt cttctgcaaa gcaatgcacg tggcccagcc |
| 301 | tgctgtggta ctggccagca gccgaggcat cgccagcttt gtgtgtgagt atgcatctcc |
| 361 | aggcaaagcc actgaggtcc gggtgacagt gcttcggcag gctgacagcc aggtgactga |
| 421 | agtctgtgcg gcaacctaca tgatggggaa tgagttgacc ttcctagatg attccatctg |
| 481 | cacgggcacc tccagtggaa atcaagtgaa cctcactatc caaggactga gggccatgga |
| 541 | cacgggactc tacatctgca aggtggagct catgtaccca ccgccatact acctgggcat |
| 601 | aggcaacgga acccagattt atgtaattga tccagaaccg tgcccagatt ctgacttcct |
| 661 | cctctggatc cttgcagcag ttagttcggg gttgtttttt tatagctttc tcctcacagc |
| 721 | tgtttctttg agcaaaatgc taaagaaaag aagccctctt acaacagggg tctatgtgaa |
| 781 | aatgccccca acagagccag aatgtgaaaa gcaatttcag ccttatttta ttcccatcaa |
| 841 | ttgagaaacc attatgaaga agagagtcca tatttcaatt tccaagagct gaggcaattc |
| 901 | taactttttt gctatccagc tatttttatt tgtttgtgca tttgggggga attcatctct |
| 961 | ctttaatata aagttggatg cggaacccaa attacgtgta ctacaattta aagcaaagga |
| 1021 | gtagaaagac agagctggga tgtttctgtc acatcagctc cactttcagt gaaagcatca |
| 1081 | cttgggatta atatggggat gcagcattat gatgtgggtc aaggaattaa gttagggaat |
| 1141 | ggcacagccc aaagaaggaa aaggcaggga gcgagggaga agactatatt gtacacacct |
| 1201 | tatatttacg tatgagacgt ttatagccga aatgatcttt tcaagttaaa ttttatgcct |
| 1261 | tttatttctt aaacaaatgt atgattacat caaggcttca aaaatactca catggctatg |
| 1321 | ttttagccag tgatgctaaa ggttgtattg catatataca tatatatata tatatatata |
| 1381 | tatatatata tatatatata tatatatata tatattttaa tttgatagta ttgtgcatag |
| 1441 | agccacgtat gtttttgtgt atttgttaat ggtttgaata taaacactat atggcagtgt |
| 1501 | ctttccacct tgggtcccag ggaagttttg tggaggagct caggacacta atacaccagg |
| 1561 | tagaacacaa ggtcatttgc taactagctt ggaaactgga tgaggtcata gcagtgcttg |
| 1621 | attgcgtgga attgtgctga gttggtgttg acatgtgctt tggggctttt acaccagttc |
| 1681 | ctttcaatgg tttgcaagga agccacagct ggtggtatct gagttgactt gacagaacac |
| 1741 | tgtcttgaag acaatggctt actccaggag acccacaggt atgaccttct aggaagctcc |
| 1801 | agttcgatgg gcccaattct tacaaacatg tggttaatgc catggacaga agaaggcagc |
| 1861 | aggtggcaga atggggtgca tgaaggtttc tgaaaattaa cactgcttgt gtttttaact |
| 1921 | caatattttc catgaaaatg caacaacatg tataatattt ttaattaaat aaaaatctgt |
| 1981 | ggtggtcgtt ttccgga |

SEQ ID NO: 5 Human CTLA4 isoform CTLA4delTM amino acid sequence
(NP_001032720.1)

| | |
|---|---|
| 1 | maclgfqrhk aqlnlatrtw pctllffllf ipvfckamhv aqpavvlass rgiasfvcey |
| 61 | aspgkatevr vtvlrqadsq vtevcaatym mgneltfldd sictgtssgn qvnltiqglr |
| 121 | amdtglyick velmypppyy lgigngtqiy viakekkpsy nrglcenapn rarm |

SEQ ID NO: 6 Human CTLA4 transcript variant 2 cDNA sequence
(NM_001037631.3; CDS 173-697)

| | |
|---|---|
| 1 | gctttctatt caagtgcctt ctgtgtgtgc acatgtgtaa tacatatctg ggatcaaagc |
| 61 | tatctatata aagtccttga ttctgtgtgg gttcaaacac atttcaaagc ttcaggatcc |
| 121 | tgaaaggttt tgctctactt cctgaagacc tgaacaccgc tcccataaag ccatggcttg |
| 181 | ccttggattt cagcggcaca aggctcagct gaacctggct accaggacct ggccctgcac |
| 241 | tctcctgttt tttcttctct tcatccctgt cttctgcaaa gcaatgcacg tggcccagcc |
| 301 | tgctgtggta ctggccagca gccgaggcat cgccagcttt gtgtgtgagt atgcatctcc |
| 361 | aggcaaagcc actgaggtcc gggtgacagt gcttcggcag gctgacagcc aggtgactga |
| 421 | agtctgtgcg gcaacctaca tgatggggaa tgagttgacc ttcctagatg attccatctg |
| 481 | cacgggcacc tccagtggaa atcaagtgaa cctcactatc caaggactga gggccatgga |
| 541 | cacgggactc tacatctgca aggtggagct catgtaccca ccgccatact acctgggcat |
| 601 | aggcaacgga acccagattt atgtaattgc taaagaaaag aagccctctt acaacagggg |
| 661 | tctatgtgaa aatgccccca acagagccag aatgtgaaaa gcaatttcag ccttatttta |
| 721 | ttcccatcaa ttgagaaacc attatgaaga agagagtcca tatttcaatt tccaagagct |
| 781 | gaggcaattc taactttttt gctatccagc tatttttatt tgtttgtgca tttgggggga |
| 841 | attcatctct ctttaatata aagttggatg cggaacccaa attacgtgta ctacaattta |
| 901 | aagcaaagga gtagaaagac agagctggga tgtttctgtc acatcagctc cactttcagt |
| 961 | gaaagcatca cttgggatta atatggggat gcagcattat gatgtgggtc aaggaattaa |
| 1021 | gttagggaat ggcacagccc aaagaaggaa aaggcaggga gcgagggaga agactatatt |
| 1081 | gtacacacct tatatttacg tatgagacgt ttatagccga aatgatcttt tcaagttaaa |
| 1141 | ttttatgcct tttatttctt aaacaaatgt atgattacat caaggcttca aaaatactca |
| 1201 | catggctatg ttttagccag tgatgctaaa ggttgtattg catatataca tatatatata |
| 1261 | tatatatata tatatatata tatatatata tatatatata tatattttaa tttgatagta |
| 1321 | ttgtgcatag agccacgtat gtttttgtgt atttgttaat ggtttgaata taaacactat |
| 1381 | atggcagtgt ctttccacct tgggtcccag ggaagttttg tggaggagct caggacacta |
| 1441 | atacaccagg tagaacacaa ggtcatttgc taactagctt ggaaactgga tgaggtcata |
| 1501 | gcagtgcttg attgcgtgga attgtgctga gttggtgttg acatgtgctt tggggctttt |
| 1561 | acaccagttc ctttcaatgg tttgcaagga agccacagct ggtggtatct gagttgactt |
| 1621 | gacagaacac tgtcttgaag acaatggctt actccaggag acccacaggt atgaccttct |
| 1681 | aggaagctcc agttcgatgg gcccaattct tacaaacatg tggttaatgc catggacaga |
| 1741 | agaaggcagc aggtggcaga atggggtgca tgaaggtttc tgaaaattaa cactgcttgt |
| 1801 | gtttttaact caatattttc catgaaaatg caacaacatg tataatattt ttaattaaat |
| 1861 | aaaaatctgt ggtggtcgtt ttccgga |

SEQ ID NO: 7 Mouse CTLA4 isoform 1 amino acid sequence (NP_033973.2)

| | |
|---|---|
| 1 | maclglrryk aqlqlpsrtw pfvalltllf ipvfseaiqv tqpsvvlass hgvasfpcey |
| 61 | spshntdevr vtvlrqtndq mtevcattft ekntvgfldy pfcsgtfnes rvnltiqglr |
| 121 | avdtglylck velmypppyf vgmgngtqiy vidpepcpds dfllwilvav slglffysfl |
| 181 | vtavslskml kkrsplttgv yvkmpptepe cekqfqpyfi pin |

SEQ ID NO: 8 Mouse CTLA4 transcript variant 1 cDNA sequence
(NM_009843.4; CDS 147-818)

| | |
|---|---|
| 1 | ctacacatat gtagcacgta ccttggatca aagctgtcta tataaagtcc ccgagtctgt |
| 61 | gtgggttcaa acacatctca aggcttctgg atcctgttgg gttttactct gctccctgag |
| 121 | gacctcagca catttgcccc ccagccatgg cttgtcttgg actccggagg tacaaagctc |
| 181 | aactgcagct gccttctagg acttggcctt ttgtagccct gctcactctt cttttcatcc |
| 241 | cagtcttctc tgaagccata caggtgaccc aaccttcagt ggtgttggct agcagccatg |
| 301 | gtgtcgccag ctttccatgt gaatattcac catcacacaa cactgatgag gtccgggtga |
| 361 | ctgtgctgcg gcagacaaat gaccaaatga ctgaggtctg tgccacgaca ttcacagaga |
| 421 | agaatacagt gggcttccta gattacccct tctgcagtgg tacctttaat gaaagcagag |
| 481 | tgaacctcac catccaagga ctgagagctg ttgacacggg actgtacctc tgcaaggtgg |
| 541 | aactcatgta cccaccgcca tactttgtgg gcatgggcaa cgggacgcag atttatgtca |
| 601 | ttgatccaga accatgcccg gattctgact tcctcctttg gatccttgtc gcagttagct |
| 661 | tggggttgtt tttttacagt ttcctggtca ctgctgtttc tttgagcaag atgctaaaga |
| 721 | aaagaagtcc tcttacaaca ggggtctatg tgaaaatgcc cccaacagag ccagaatgtg |
| 781 | aaaagcaatt tcagccttat tttattccca tcaactgaaa ggccgtttat gaagaagaag |
| 841 | gagcatactt cagtctctaa aagctgaggc aatttcaact ttccttttct ctccagctat |
| 901 | ttttacctgt ttgtatattt taaggagagt atgcctctct ttaatagaaa gctggatgca |
| 961 | aaattccaat taagcatact acaatttaaa gctaaggagc atgaacagag agctgggata |
| 1021 | tttctgttgt gtcagaacca ttttactaaa agcatcactt ggaagcagca taaggatata |
| 1081 | gcattatggt gtggggtcaa gggaacatta gggaatggca cagcccaaag aaaggaaggg |
| 1141 | ggtgaaggaa gagattatat tgtacacatc ttgtatttac ctgagagatg tttatgactt |
| 1201 | aaataatttt taaatttttc atgctgttat tttctttaac aatgtataat tacacgaagg |
| 1261 | tttaaacatt tattcacaga gctatgtgac atagccagtg gttccaaagg ttgtagtgtt |
| 1321 | ccaagatgta tttttaagta atattgtaca tgggtgtttc atgtgctgtt gtgtatttgc |
| 1381 | tggtggtttg aatataaaca ctatgtatca gtgtcgtccc acagtgggtc ctggggaggt |
| 1441 | ttggctgggg agcttaggac actaatccat caggttggac tcgaggtcct gcaccaactg |
| 1501 | gcttggaaac tagatgaggc tgtcacaggg ctcagttgca taaaccgatg gtgatggagt |
| 1561 | gtaaactggg tctttacact cattttattt tttgtttctg cttttgtttt cttcaatgat |
| 1621 | ttgcaaggaa accaaaagct ggcagtgttt gtatgaacct gacagaacac tgtcttcaag |
| 1681 | gaaatgcctc attcctgaga ccagtaggtt tgttttttta ggaagttcca atactaggac |
| 1741 | cccctacaag tactatggct cctcgaaaac acaaagttaa tgccacagga agcagcagat |
| 1801 | ggtaggatgg gatgcacaag agttcctgaa aactaacact gttagtgttt tttttttaac |
| 1861 | tcaatatttt ccatgaaaat gcaaccacat gtataatatt tttaattaaa taaaagtttc |
| 1921 | ttgtgattgt ttt |

*Included in Table 1 are RNA nucleic acid molecules (e.g., thymidines replaced with uridines), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid.
*Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide.
*Included in Table 1 are other known CTLA4 nucleic acid and amino acid sequences.

Ipilimumab, sold under the brand name Yervoy, is a monoclonal antibody medication that works to activate the immune system by targeting CTLA4, a protein receptor that downregulates the immune system. Ipilimumab blocks the interaction between CTLA4 and its ligands. As described above, cytotoxic T lymphocytes (CTLs) can recognize and destroy cancer cells. However, an inhibitory mechanism interrupts this destruction. Ipilimumab turns off this inhibitory mechanism and boosts the body's immune response against cancer cells.

Ipilimumab binds to Epitope 1 (¹³⁴MYPPPY¹³⁹ (SEQ ID NO: 1)) of CTLA4. The MYPPPY motif (SEQ ID NO: 1), including Tyr139, is highly conserved across both CTLA4 and its immune stimulatory paralog CD28, and interfaces directly with both CD80 and with the CTLA4 inhibitor ipilimumab. Other antibodies, e.g., murine 26 antibody or humanized 121 antibody described herein and in U.S. Pat. No. 7,034,121 B2 do not bind to Epitope 1. Another CTLA4 epitope, Epitope 2 (⁶⁵SICT⁶⁸ (SEQ ID NO: 2), is known in the art.

Ipilimumab was approved by the US Food and Drug Administration (FDA) for treatment of melanoma (e.g., unresectable or metastatic melanoma in adults and pediatric patients), renal cell carcinoma (RCC), colorectal cancer, hepatocellular carcinoma, non-small cell lung cancer (NSCLC), and malignant pleural mesothelioma. Ipilimumab is also effective in combination with nivolumab that targets PD-1.

While effective, a major drawback of ipilimumab therapy is its association with severe and potentially fatal immunological adverse effects due to T cell activation and proliferation, occurring in ten to twenty percent of patients. Serious adverse effects include stomach pain, bloating, constipation, diarrhea, fever, trouble breathing, and urinating problems. Between 5.7 and 9.1% of individuals treated with ipilimumab develop checkpoint inhibitor induced colitis. Individual cases of severe neurologic disorders following ipilimumab have been observed, including acute inflammatory demyelination polyneuropathy and an ascending motor paralysis, and myasthenia gravis.

### Antigen-Binding Proteins

Provided herein are antigen-binding proteins that bind to CTLA4. The antigen-binding proteins of the present disclosure can take any one of many forms of antigen-binding proteins known in the art. In various embodiments, the antigen-binding proteins of the present disclosure take the form of an antibody, or antigen-binding antibody fragment, or an antibody protein product.

In various embodiments of the present disclosure, the antigen-binding protein comprises, consists essentially of, or consists of an antibody or a fragment thereof. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. Antibody-based antigen-binding proteins comprise the CDRs of the antibody, but not necessarily other regions (e.g., the constant region). The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra).

CDR refers to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5^{th} Edition, U.S. Department of Health and Human Services, 1992; Chothia et al. (1987) J. Mol. Biol. 196, 901; and MacCallum et al., J. Mol. Biol. (1996)262, 732, each of which is incorporated by reference in its entirety).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

An antibody can be cleaved into fragments by enzymes, such as, e.g., papain and pepsin. Papain cleaves an antibody to produce two Fab′ fragments and a single Fc fragment. Pepsin cleaves an antibody to produce a F(ab′)₂ fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab′ fragment or a F(ab′)2 fragment.

The architecture of antibodies has been exploited to create a growing range of alternative antibody formats that spans a molecular-weight range of at least about 12-150 kDa and has a valency (n) range from monomeric (n=1), to dimeric (n=2), to trimeric (n=3), to tetrameric (n=4), and potentially higher; such alternative antibody formats are referred to herein as “antibody protein products.” Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant I domains are added to the V regions to generate a Fab′ fragment. Both scFv and Fab′ fragments can be easily produced in host cells, e.g., prokaryotic host cells. Other antibody protein products include disulfide-bond stabilized scFv (ds-scFv), single chain Fab′ (scFab′), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4 (5): 586-591 (2012).

Other antibody protein products include a single chain antibody (SCA); a diabody; a triabody; a tetrabody, and the like.

In various aspects, the antigen-binding protein of the present disclosure comprises, consists essentially of, or consists of any one of these antibody protein products. In various aspects, the antigen-binding protein of the present disclosure comprises, consists essentially of, or consists of any one of an scFv, Fab′, F(ab′)2, VHH/VH, Fv fragment, ds-scFv, scFab′, half antibody-scFv, heterodimeric Fab/scFv-Fc, heterodimeric scFv-Fc, heterodimeric IgG (CrossMab), tandem scFv, tandem biparatopic scFv, Fab/scFv-Fc, tandem Fab′, single-chain diabody, dimeric antibody, multimeric antibody (e.g., a diabody, triabody, tetrabody), miniAb, peptibody VHH/VH of camelid heavy chain antibody, sdAb, diabody (single-chain diabody, homodimeric diabody, heterodimeric diabody, tandem diabody (TandAb), diabody that self-dimerizes), a triabody, a tetrabody. An ordinarily skilled artisan would understand that any bispecific antigen-binding protein formats can be used to generate biparatopic antigen-binding protein formats. In some embodiments, the antigen-binding protein is a dual-affinity re-targeting antibody (DART). In some embodiments, the antigen-binding protein is a bispecific T-cell engager (BiTE).

In various aspects, the antigen-binding protein of the present disclosure is linked to an agent. As described below, the agent may be any known in the art, including, but not limited to, chemotherapeutic agents, cytokines and growth factors, cytotoxic agents, detectable agent (e.g., fluorescein), and the like.

The antigen-binding proteins provided herein bind to CTLA4 in a non-covalent and reversible manner. In various embodiments, the binding strength of the antigen-binding protein to CTLA4 may be described in terms of its affinity, a measure of the strength of interaction between the binding site of the antigen-binding protein and the epitope. In various aspects, the antigen-binding proteins provided herein have high-affinity for CTLA4 and thus will bind a greater amount of CTLA4 in a shorter period of time than low-affinity antigen-binding proteins. In various aspects, the antigen-binding protein has an equilibrium association constant, K_{A}, which is at least 10⁵ mol⁻¹, at least 10⁶ mol⁻¹, at least 10⁷ mol⁻¹, at least 10⁸ mol⁻¹, at least 10⁹ mol⁻¹, or at least 10¹⁰ mol⁻¹. As understood by the artisan of ordinary skill, K_{A} can be influenced by factors including pH, temperature and buffer composition.

In various embodiments, the binding strength of the antigen-binding protein to CTLA4 may be described in terms of its sensitivity. K_{D} is the equilibrium dissociation constant, a ratio of k_{off}/kₒₙ, between the antigen-binding protein and CTLA4. K_{D} and K_{A} are inversely related. The K_{D} value relates to the concentration of the antigen-binding protein (the amount of antigen-binding protein needed for a particular experiment) and so the lower the K_{D} value (lower concentration) the higher the affinity of the antigen-binding protein. In various aspects, the binding strength of the antigen-binding protein to CTLA4 may be described in terms of K_{D}. In various aspects, the K_{D} of the antigen-binding proteins provided herein is about 10⁻¹, about 10⁻², about 10⁻³, about 10⁻⁴, about 10⁻⁵, about 10⁻⁶, or less. In various aspects, the K_{D} of the antigen-binding proteins provided herein is micromolar, nanomolar, picomolar or femtomolar. In various aspects, the K_{D} of the antigen-binding proteins provided herein is within a range of about 10⁻⁴ to 10⁻⁶ or 10⁻⁷ to 10⁻⁹ or 10⁻¹⁰ to 10⁻¹² or 10⁻¹³ to 10⁻¹⁵. In various aspects, the K_{D} of the antigen-binding proteins provided herein is within a range of about 1.0×10⁻¹² M to about 1.0×10⁻⁸ M. In various aspects, the K_{D} of the antigen-binding proteins is within a range of about 1.0×10⁻¹¹ M to about 1.0×10⁻⁹ M.

In various aspects, the affinity of the antigen-binding proteins are measured or ranked using a flow cytometry- or Fluorescence-Activated Cell Sorting (FACS)-based assay. Flow cytometry-based binding assays are known in the art. See, e.g., Cedeno-Arias et al., Sci Pharm 79 (3): 569-581 (2011); Rathanaswami et al., Analytical Biochem 373:52-60 (2008); and Geuijen et al., J Immunol Methods 302 (1-2): 68-77 (2005). In various aspects, the affinity of the antigen-binding proteins are measured or ranked using a competition assay as described in Trikha et al., Int J Cancer 110:326-335 (2004) and Tam et al., Circulation 98 (11): 1085-1091 (1998), as well as below.

Avidity gives a measure of the overall strength of an antigen-binding protein-antigen complex. It is dependent on three major parameters: affinity of the antigen-binding protein for the epitope, valency of both the antigen-binding protein and CTLA4, and structural arrangement of the parts that interact. The greater an antigen-binding protein's valency (number of antigen binding sites), the greater the amount of antigen (CTLA4) it can bind. In various aspects, the antigen-binding proteins have a strong avidity for CTLA4. In various aspects, the antigen-binding proteins are multivalent. In various aspects, the antigen-binding proteins are bivalent. In various instances, the antigen antigen-binding proteins are monovalent.

### Sequence Identity/Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The percent identity between two sequences is a function of the number of identical positions shared by the sequences (i.e., % identity=#of identical positions/total#of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the World Wide Web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25 (17): 3389 3402. When utilizing BLAST and Gapped BLAST programs, the default parameters of the respective programs (e.g., XBLAST and NBLAST) can be used (available on the World Wide Web at the NCBI website).

### Methods of Antibody Production and Related Methods

Suitable methods of making antigen-binding proteins (e.g., antibodies, antigen-binding antibody fragments, and antibody protein products) are known in the art. For instance, standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, NY (2001)).

Depending on the host species, various adjuvants can be used to increase the immunological response leading to greater antibody production by the host. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Other methods of antibody production are summarized in Table 2.

TABLE 2
Alternative Methods of Antibody Production

| Technique | References |
|---|---|
| EBV-hybridoma methods and | Haskard and Archer, J. Immunol. Methods, |
| Bacteriophage vector expression | 74(2), 361-67 (1984), Roder et al., Methods |
| systems | Enzymol., 121, 140-67 (1986), and Huse et al., |
| | Science, 246, 1275-81 (1989)). |
| Methods of producing antibodies | U.S. Pat. Nos. 5,545,806, 5,569,825, and |
| in non-human animals | 5,714,352, and U.S. Patent Application |
| | Publication No. 2002/0197266 |
| inducing in vivo production in | Orlandi et al. (Proc Natl Acad Sci 86: 3833- |
| the lymphocyte population or by | 3837; 1989), and Winter G and Milstein C |
| screening recombinant | (Nature 349: 293-299, 1991). |
| immunoglobulin libraries or | |
| panels of highly specific | |
| binding reagents | |
| Methods of producing | Protein production and purification” Nat |
| recombinant proteins | Methods 5(2): 135-146 (2008). |
| Phage display | Janeway et al., supra, Huse et al., supra, and |
| | U.S. Pat. No. 6,265,150). Related methods also |
| | are described in U.S. Pat. No. 5,403,484; |
| | U.S. Pat. No. 5,571,698; U.S. Pat. No. |
| | 5,837,500; U.S. Pat. No. 5,702,892. The |
| | techniques described in U.S. Pat. No. |
| | 5,780,279; U.S. Pat. No. 5,821,047; |
| | U.S. Pat. No. 5,824,520; U.S. Pat. No. 5,855,885; |
| | U.S. Pat. No. 5,858,657; U.S. Pat. No. |
| | 5,871,907; U.S. Pat. No. 5,969,108; |
| | U.S. Pat. No. 6,057,098; and U.S. Pat. No. 6,225,447 |
| Antibodies can be produced by | U.S. Pat. Nos. 5,545,806 and 5,569,825, and |
| transgenic mice | Janeway et al., supra. |

### Antibody Engineering to Improve Pharmacokinetics (PK)

An antigen-binding protein can be engineered to increase or improve its pharmacokinetic (PK) properties (e.g., half-life). Numerous properties of an antigen-binding protein can influence pharmacokinetics including, but not limited to, molecular size, folding stability, solubility, target interaction, neonatal Fc binding capacity, and charge. Modifications to the antigen-binding protein include, but are not limited to antigen-binding domain conjugation to one or more carrier proteins, PEGylation, acylation (e.g., by conjugation to a fatty acid molecule), polysialylation, or glycosylation. Amino acid sequence modifications can be used to improve or optimize the PK properties of the protein, and conjugation to large, slowly metabolized macromolecules can also modify the PK properties of the protein. Macromolecules that can be conjugated to the antigen protein include, but are not limited to, proteins (e.g., albumin or albumin-binding protein; such can also be expressed as a fusion protein), polysaccharides (e.g., sepharose, agarose, cellulose, or cellulose beads), polymeric amino acids (polyglutamic acid or polylysine), amino acid copolymers, inactivated virus particles, inactivated bacterial toxins (e.g., leukotoxin or diphtheria, tetanus, or cholera toxins or molecules), inactivated bacteria, dendritic cells, thyroglobulin, polyamino acids (e.g., poly(D-lysine: D-glutamic acid)), VP6 polypeptides of rotaviruses, influenza virus hemaglutinin, influenza virus nucleoprotein, Keyhole Limpet Hemocyanin (KLH), and hepatitis B virus core protein and surface antigen (WO2021146436). Additional PK modulators known in the art include lipophiles, bile acids, steroids, phospholipid analogues, and vitamins, examples of which include, but are not limited to, cholesterol, fatty acids, cholic acid, lithocholic acid, dialkylglycerides, diacylglyceride, phospholipids, sphingolipids, naproxen, ibuprofen, vitamin E, and biotin (U.S. Pat. No. 9,322,018). Methods for producing modified antigen-binding proteins as described herein are known in the art.

In some embodiments, the antigen-binding protein receptor is fused or otherwise linked to a conventional fragment crystallizable region (Fc Region) or a fragment thereof. For example, the Fc region can be an IgG1, IgG2, IgG3, or IgG4 Fc region. In some embodiments, mutations in the Fc region of the antigen-binding protein can be engineered to modulate its interaction with the neonatal Fc receptor (FcRn), which is involved in receptor-mediated internalization and recycling of IgG occur via FcRn (Sockolosky and Szoka, Adv Drug Deliv Rev. (2015) 109-24), thereby improving its pharmacokinetic properties (US20210277092). For example, the Fc region can comprise a LALAPG amino acid sequence that inhibits binding of the antigen-binding protein to the neonatal Fcγ receptor. In some embodiments, the antigen-binding protein is fused or otherwise linked to an albumin-binding protein.

In addition to a conventional Fc region or a fragment thereof, there are engineered FcRN binding peptides, when fused to a protein, that significantly enhance the half-life of the protein in primates (see e.g., Datta-Mannan et al. (2018) Biotechnology Journal, 14 (3): e1800007; Mezo et al. (2008) Proc Natl Acad Sci U.S.A., 105 (7): 2337-2342; Sockolosky et al. (2012) Proc Natl Acad Sci U.S.A., 109 (40): 16095-16100; each of which is incorporated by reference). such peptides Ile small linear and cyclic FcRn binding peptides (collectively called FcRnBPs) that can be fused to a combination of the N- and C-termini of a protein, e.g., Fab, to improve the pharmacokinetics of the protein. Such peptides include those having an exemplary aminio acid sequence of QRFCTGHFGGLYPCNG; QRFCTGHFGGLHPCNG; QRFVTGHFGGLYPANG; or QRFVTGHFGGLHPANG.

In some embodiments, the macromolecule is directly conjugated to the antigen-binding protein. In some embodiments, the macromolecule is fused to the antigen-binding peptide via a linker.

Modified antigen-binding proteins as described herein can have improved or optimized pharmacokinetic (PK) properties, for example, a plasma half-life in a human subject of greater than 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 10 days, 14 days, or 30 days.

Methods of testing the antigen-binding protein for the ability to bind to the epitope(s) of CTLA4 regardless of how the antigen-binding proteins are produced are known in the art and include any binding assay, such as, for example, radioimmunoassay (RIA), ELISA, Western blot, immunoprecipitation, SPR, and competitive inhibition assays (see, e.g., U.S. Patent Application Publication No. 2002/0197266).

### Fusion Polypeptides or Modified Antigen-Binding Proteins

The present invention also provides chimeric or fusion proteins comprising an antigen-binding protein disclosed herein. Said proteins may comprise an antigen-binding protein or an antigen-binding fragment thereof operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide corresponding to a marker of the present invention is fused to the carboxyl terminus of GST sequences. Such fusion proteins can facilitate the purification of a recombinant polypeptide of the present invention.

In some embodiments, the fusion protein contains a heterologous signal sequence, immunoglobulin fusion protein, enzyme (e.g., a horse radish peroxidase, etc.), toxin, or other useful protein sequence (e.g., other tags known in the art (e.g., HA tag, myc tag, GFP, etc.). Chimeric and fusion proteins of the present invention can be produced by standard recombinant DNA techniques. In some embodiments, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide of the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement to or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least or about 50%, and preferably at least or about 75%, at least or about 90%, or at least or about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It Is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

TABLE 3
Representative sequences of exemplary CTLA4-binding proteins or
fragments thereof

SEQ ID NO: 9 Heavy chain variable domain (VH) of the 26 antibody-amino acid sequence

| | |
|---|---|
| 1 | MDVLVLFLCL VAFPSCVLSQ VQLKESGPGL VAPSQSLSIT CTVSGFSLTS YGVYWVRQPP |
| 61 | GKGLEWLGVI WAGGTTNYNS ALMSRLSISK DNSKSQVFLK MSSLQTDDTA MYYCARGPPH |
| 121 | AMMKRGYAMD YWGQGTSVIV SS |

SEQ ID NO: 10 Light chain variable domain (VL) of the 26 antibody-amino acid sequence

| | |
|---|---|
| 1 | MDFQVQIFSF LLISASVILS RGQNVLTQSP AIMPASPGEK VTMTCSATSS ITYMSWYQQK |
| 61 | SGSSPRLLIY DTSNLASGVP VRFSGSGSGT SYSLTISRME AEDAATYYCQ QWSSYPLTFG |
| 121 | AGTKLELK |

SEQ ID NO: 11 Light chain variable domain (VL) of the 26 antibody-cDNA sequence

| | |
|---|---|
| 1 | atggattttc aagtgcagat tttcagcttc ctgctaatca gtgcctcagt catactgtcc |
| 61 | agaggacaaa atgttctcac ccagtctcca gcaatcatgc ctgcatctcc aggggagaag |
| 121 | gtcaccatga cctgcagtgc cacctcaagt ataacttaca tgtcctggta ccagcagaag |
| 181 | tcaggatcct cccccagact cctgatttat gacacatcca acctggcttc tggagtccct |
| 241 | gttcgcttca gtggcagtgg gtctgggacc tcttactctc tcacaatcag ccgaatggag |
| 301 | gctgaagatg ctgccactta ttactgccag cagtggagta gttacccgct cacgttcggt |
| 361 | gctgggacca agctggagct gaaa |

SEQ ID NO: 12 Heavy chain variable domain (VH) of the 121 antibody-amino acid sequence

| | |
|---|---|
| 1 | QVQLQESGPG LVKPSQTLSL TCTVSGFSLT SYGVYWVRQP PGKGLEWLGV IWAGGTTNYN |
| 61 | SALMSRLTIS KDTSKNQVSL KLSSVTAADT AVYYCARGPP HAMMKRGYAM DYWGQGTLVT |
| 121 | VSS |

SEQ ID NO: 13 Heavy chain variable domain (VH) of the 121 antibody-cDNA sequence

| | |
|---|---|
| 1 | caggtgcagc tgcaagagtc aggacctggc ctggtgaagc cctcacagac actgtccttg |
| 61 | acttgcactg tctctgggtt ttcattaacc tcatatggtg tatattgggt tcgccagcct |
| 121 | ccaggaaagg gtctggagtg gctgggagta atatgggctg gtggtaccac aaattataat |
| 181 | tcggctctca tgtccagact gacaatcagc aaagacacat ccaagaacca agtttcctta |
| 241 | aaactcagca gtgtgactgc agcggacaca gccgtctact actgtgcccg aggccccccg |
| 301 | cacgctatga tgaagagagg ctatgctatg gactactggg gacaaggaac cctagtcaca |
| 361 | gtctcctcag g |

SEQ ID NO: 14 Light chain variable domain (VL) of the 121 antibody-amino acid sequence

| | |
|---|---|
| 1 | DIQMTQSPSS LSASVGDRVT ITCSATSSIT YMSWYQQKPG KAPKLLIYDT SNLASGVPSR |
| 61 | FSGSGSGTDY TLTISSLQPE DFATYYCQQW SSYPLTFGGG TKLEIK |

SEQ ID NO: 15 Heavy chain variable domain (VH) of ipilimumab-amino acid

| | |
|---|---|
| 1 | QVQLVESGGG VVQPGRSLRL SCAASGFTFS SYTMHWVRQA PGKGLEWVTF ISYDGNNKYY |
| 61 | ADSVKGRFTI SRDNSKNTLY LQMNSLRAED TAIYYCARTG WLGPFDYWGQ GTLVTVSS |

SEQ ID NO: 16 Heavy chain variable domain (VH) of ipilimumab-cDNA

| | |
|---|---|
| 1 | caggtgcagc tggtggagtc tgggggaggc gtggtccagc ctgggaggtc cctgagactc |
| 61 | tcctgtgcag cctctggatt caccttcagt agctatacta tgcactgggt ccgccaggct |
| 121 | ccaggcaagg ggctggagtg ggtgacattt atatcatatg atggaaacaa taaatactac |
| 181 | gcagactccg tgaagggccg attcaccatc tccagagaca attccaagaa cacgctgtat |
| 241 | ctgcaaatga acagcctgag agctgaggac acggctatat attactgtgc gaggaccggc |
| 301 | tggctggggc cctttgacta ctggggccag ggaaccctgg tcaccgtctc ctcag |

SEQ ID NO: 17 Light chain variable domain (VL) of ipilimumab-amino acid

| | |
|---|---|
| 1 | EIVLTQSPGT LSLSPGERAT LSCRASQSVG SSYLAWYQQK PGQAPRLLIY GAFSRATGIP |
| 61 | DRFSGSGSGT DFTLTISRLE PEDFAVYYCQ QYGSSPWTFG QGTKVEIK |

SEQ ID NO: 18 Light chain variable domain (VL) of ipilimumab-cDNA sequence

| | |
|---|---|
| 1 | gaaattgtgt tgacgcagtc tccaggcacc ctgtctttgt ctccagggga aagagccacc |
| 61 | ctctcctgca gggccagtca gagtgttggc agcagctact tagcctggta ccagcagaaa |
| 121 | cctggccagg ctcccaggct cctcatctat ggtgcattca gcagggccac tggcatccca |
| 181 | gacaggttca gtggcagtgg gtctgggaca gacttcactc tcaccatcag cagactggag |
| 241 | cctgaagatt ttgcagtgta ttactgtcag cagtatggta gctcaccgtg gacgttcggc |
| 301 | caagggacca aggtggaaat caaac |

SEQ ID NO: 19 BioE2021 (ipilimumab ScFv) amino acid sequence

| | |
|---|---|
| 1 | EIVLTQSPGT LSLSPGERAT LSCRASQSVG SSYLAWYQQK PGQAPRLLIY GAFSRATGIP |
| 61 | DRFSGSGSGT DFTLTISRLE PEDFAVYYCQ QYGSSPWTFG QGTKVEIKGG GGSGGGGSGG |
| 121 | GGSQVQLVES GGGVVQPGRS LRLSCAASGF TFSSYTMHWV RQAPGKGLEW VTFISYDGNN |
| 181 | KYYADSVKGR FTISRDNSKN TLYLQMNSLR AEDTAIYYCA RTGWLGPFDY WGQGTLVTVS |
| 241 | S |

SEQ ID NO: 20 BioE2031 (121 ScFv) amino acid sequence

| | |
|---|---|
| 1 | DIQMTQSPSS LSASVGDRVT ITCSATSSIT YMSWYQQKPG KAPKLLIYDT SNLASGVPSR |
| 61 | FSGSGSGTDY TLTISSLQPE DFATYYCQQW SSYPLTFGGG TKVEIKGGGG SGGGGSGGGG |
| 121 | SQVQLQESGP GLVKPSQTLS LTCTVSGFSL TSYGVYWVRQ PPGKGLEWLG VIWAGGTTNY |
| 181 | NSALMSRLTI SKDTSKNQVS LKLSSVTAAD TAVYYCARGP PHAMMKRGYA MDYWGQGTLV |
| 241 | TVSS |

SEQ ID NO: 21

| | |
|---|---|
| 1 | QVQLQESGPG LVKPSQTLSL TCTVSGFSLT SYGVYWVRQP PGKGLEWLGV IWAGGTTNYN |
| 61 | SALMSRLTIS KDTSKNQVSL KLSSVTAADT AVYYCARGPP HAMMKRGYAM DYWGQGTLVT |
| 121 | VSSASTKGPS VFPLAPSSKS TSGGTAALGC LVKDYFPEPV TVSWNSGALT SGVHTFPAVL |
| 181 | QSSGLYSLSS VVTVPSSSLG TQTYICNVNH KPSNTKVDKK V |

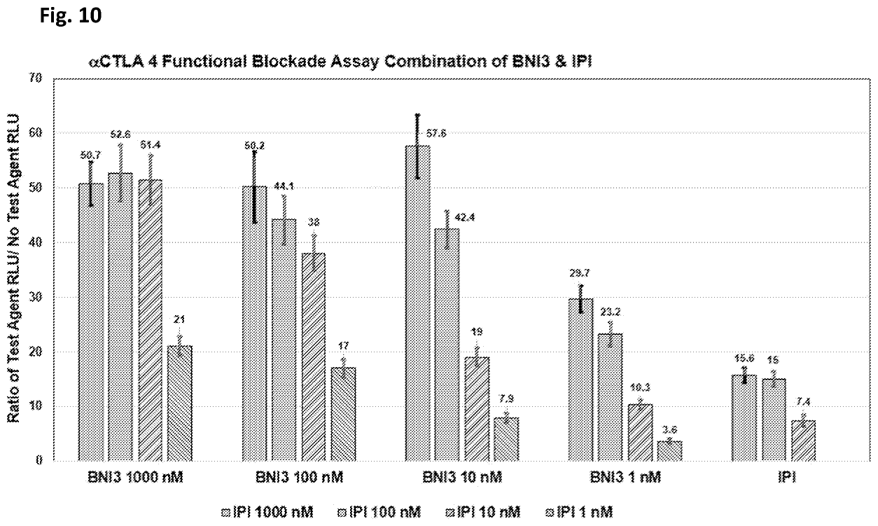

*SEQ ID NO: 21 optionally comprises the sequence (EPKSCDKT) at the C-terminal end,
which corresponds to the hinge region for F(ab')2 or Fab', where the cysteine residue forms
inter-chain bond with kappa light. Alternatively, F(ab')2 may comprise the sequence (e.g.,
proprietary sequence as indicated in FIG. 101) that forms inter-chain dimerization domain
C-terminal to SEQ ID NO: 21.
*See Table 3 and FIGS. 10F and 10I.
SEQ ID NO: 22 BioE2034 121 VL and CL amino acid sequence

| | |
|---|---|
| 1 | DIQMTQSPSS LSASVGDRVT ITCSATSSIT YMSWYQQKPG KAPKLLIYDT SNLASGVPSR |
| 61 | FSGSGSGTDY TLTISSLQPE DFATYYCQQW SSYPLTFGGG TKLEIKRTVA APSVFIFPPS |
| 121 | DEQLKSGTAS VVCLLNNFYP REAKVQWKVD NALQSGNSQE SVTEQDSKDS TYSLSSTLTL |
| 181 | SKADYEKHKV YACEVTHQGL SSPVTKSFNR GEC |

SEQ ID NO: 23 BioE2051 (anti-CTLA4 diabody => single-chain diabody; self-
demerizes; tandem diabody (TandAb)) amino acid sequence

| | |
|---|---|
| 1 | QVQLQESGPG LVKPSQTLSL TCTVSGFSLT SYGVYWVRQP PGKGLEWLGV IWAGGTTNYN |
| 61 | SALMSRLTIS KDTSKNQVSL KLSSVTAADT AVYYCARGPP HAMMKRGYAM DYWGQGTLVT |
| 121 | VSSGGSGGSG GSEIVLTQSP GTLSLSPGER ATLSCRASQS VGSSYLAWYQ QKPGQAPRLL |
| 181 | IYGAFSRATG IPDRFSGSGS GTDFTLTISR LEPEDFAVYY CQQYGSSPWT FGQGTKVEIK |
| 241 | GGSGGSGGSQ VQLVESGGGV VQPGRSLRLS CAASGFTFSS YTMHWVRQAP GKGLEWVTFI |
| 301 | SYDGNNKYYA DSVKGRFTIS RDNSKNTLYL QMNSLRAEDT AIYYCARTGW LGPFDYWGQG |
| 361 | TLVTVSSGGS GGSGGSDIQM TQSPSSLSAS VGDRVTITCS ATSSITYMSW YQQKPGKAPK |
| 421 | LLIYDTSNLA SGVPSRFSGS GSGTDYTLTI SSLQPEDFAT YYCQQWSSYP LTFGGGTKVE |
| 481 | IK |

SEQ ID NO: 24 BioE2052 (anti-CTLA4 diabody => single-chain diabody; self-
dimerizes; tandem diabody (TandAb)) amino acid sequence

| | |
|---|---|
| 1 | QVQLVESGGG VVQPGRSLRL SCAASGFTFS SYTMHWVRQA PGKGLEWVTF ISYDGNNKYY |
| 61 | ADSVKGRFTI SRDNSKNTLY LQMNSLRAED TAIYYCARTG WLGPFDYWGQ GTLVTVSSGG |
| 121 | SGGSGGSDIQ MTQSPSSLSA SVGDRVTITC SATSSITYMS WYQQKPGKAP KLLIYDTSNL |
| 181 | ASGVPSRFSG SGSGTDYTLT ISSLQPEDFA TYYCQQWSSY PLTFGGGTKV EIKGGSGGSG |
| 241 | GSQVQLQESG PGLVKPSQTL SLTCTVSGFS LTSYGVYWVR QPPGKGLEWL GVIWAGGTTN |
| 301 | YNSALMSRLT ISKDTSKNQV SLKLSSVTAA DTAVYYCARG PPHAMMKRGY AMDYWGQGTL |
| 361 | VTVSSGGSGG SGGSEIVLTQ SPGTLSLSPG ERATLSCRAS QSVGSSYLAW YQQKPGQAPR |
| 421 | LLIYGAFSRA TGIPDRFSGS GSGTDFTLTI SRLEPEDFAV YYCQQYGSSP WTFGQGTKVE |
| 481 | IK |

SEQ ID NO: 25 BioE2051 (anti-CTLA4 diabody => single-chain diabody; self-
dimerizes; tandem diabody (TandAb)) cDNA sequence

| | |
|---|---|
| 1 | CAAGTGCAGC TCCAAGAGTC CGGCCCCGGC CTCGTGAAAC CCAGCCAGAC ACTGTCTCTG |
| 61 | ACATGCACCG TGAGCGGCTT TTCTCTGACC AGCTACGGAG TGTATTGGGT GAGACAACCC |
| 121 | CCCGGCAAGG GACTGGAGTG GCTGGGAGTG ATTTGGGCCG GCGGCACCAC CAACTACAAT |
| 181 | AGCGCCCTCA TGTCTAGACT CACCATCTCC AAGGACACCA GCAAGAACCA AGTGTCCCTC |
| 241 | AAGCTGTCCA GCGTCACAGC TGCCGACACC GCCGTGTACT ATTGTGCTAG AGGCCCCCCC |
| 301 | CATGCCATGA TGAAGAGAGG CTATGCCATG GATTACTGGG GCCAAGGCAC ACTGGTGACC |
| 361 | GTCAGCTCCG GAGGAAGCGG CGGCAGCGGA GGCTCCGAAA TTGTGCTCAC CCAGAGCCCC |
| 421 | GGCACACTGT CTCTGAGCCC CGGCGAAAGG GCCACACTGA GCTGCAGAGC CTCCCAATCC |
| 481 | GTGGGCAGCA GCTATCTGGC TTGGTATCAG CAGAAACCCG GCCAAGCCCC TAGACTGCTG |
| 541 | ATCTATGGAG CCTTTTCTAG AGCTACCGGC ATCCCCGACA GATTCTCCGG CAGCGGCAGC |
| 601 | GGCACAGACT TTACACTGAC AATTTCTAGA CTGGAACCAG AGGATTTCGC CGTCTACTAC |
| 661 | TGCCAGCAGT ATGGAAGCAG CCCTTGGACC TTTGGCCAAG GCACCAAGGT GGAGATCAAG |
| 721 | GGAGGAAGCG GAGGCAGCGG AGGCAGCCAA GTGCAGCTCG TGGAAAGCGG AGGAGGCGTG |
| 781 | GTGCAGCCCG GCAGATCCCT CAGACTGAGC TGCGCCGCCA GCGGCTTCAC CTTCAGCTCC |
| 841 | TATACCATGC ACTGGGTGAG GCAAGCCCCC GGCAAAGGAC TGGAGTGGGT CACCTTCATC |
| 901 | AGCTACGACG GCAACAACAA GTACTACGCC GACAGCGTGA AGGGAAGGTT CACCATCTCT |
| 961 | AGAGACAACT CCAAGAACAC CCTCTACCTC CAGATGAACT CTCTGAGGGC CGAAGACACC |
| 1021 | GCCATCTACT ACTGCGCTAG AACCGGCTGG CTGGGACCCT TTGACTACTG GGGACAAGGC |
| 1081 | ACACTGGTCA CAGTGTCCTC CGGAGGAAGC GGAGGCTCCG GCGGCAGCGA TATCCAGATG |
| 1141 | ACCCAATCCC CTTCCTCTCT GAGCGCCTCC GTGGGAGATA GGGTCACCAT TACATGTAGC |
| 1201 | GCCACAAGCA GCATCACCTA CATGAGCTGG TACCAGCAGA AACCCGGAAA GGCCCCTAAG |
| 1261 | CTGCTCATCT ACGACACCTC CAATCTGGCC AGCGGCGTGC CTTCTAGATT TAGCGGCTCC |
| 1321 | GGAAGCGGCA CAGATTACAC ACTGACAATC AGCTCTCTGC AGCCAGAGGA CTTCGCCACC |
| 1381 | TACTACTGTC AGCAGTGGAG CAGCTACCCT CTGACCTTTG GCGGCGGCAC CAAGGTGGAA |
| 1441 | ATCAAACACC ACCATCACCA CCATCACCAC CATCAC |

SEQ ID NO: 26 BioE2052 (anti-CTLA4 diabody => single-chain diabody; self-
dimerizes; tandem diabody (TandAb)) cDNA sequence

| | |
|---|---|
| 1 | CAAGTGCAGC TCGTGGAATC CGGCGGAGGA GTCGTGCAGC CCGGCAGAAG CCTCAGACTG |
| 61 | AGCTGCGCCG CCAGCGGATT CACCTTCAGC AGCTACACCA TGCACTGGGT GAGGCAAGCC |
| 121 | CCCGGCAAAG GACTGGAGTG GGTCACATTC ATCTCCTACG ATGGCAACAA CAAGTACTAC |
| 181 | GCCGACAGCG TGAAGGGAAG GTTTACCATC TCTAGAGATA ACTCCAAGAA CACCCTCTAC |
| 241 | CTCCAGATGA ACTCTCTGAG AGCTGAGGAC ACAGCCATCT ATTACTGCGC TAGAACCGGA |
| 301 | TGGCTGGGCC CTTTCGACTA CTGGGGACAA GGCACACTGG TGACAGTGTC CTCCGGAGGC |
| 361 | AGCGGAGGCA GCGGAGGCAG CGACATCCAG ATGACCCAAT CCCCTAGCTC TCTGAGCGCC |
| 421 | AGCGTGGGCG ATAGAGTGAC CATTACATGC TCCGCCACCA GCAGCATCAC CTACATGAGC |
| 481 | TGGTACCAGC AAAAGCCCGG CAAAGCCCCC AAGCTGCTGA TCTACGATAC CAGCAATCTG |
| 541 | GCCAGCGGCG TGCCTTCTAG ATTTTCCGGC TCCGGAAGCG GCACCGATTA CACACTGACC |
| 601 | ATTTCCTCTC TGCAGCCAGA GGATTTCGCC ACCTACTATT GCCAGCAGTG GTCCTCCTAC |
| 661 | CCTCTCACCT TTGGCGGCGG AACAAAGGTG GAGATCAAAG GCGGCAGCGG CGGCTCCGGA |
| 721 | GGCAGCCAAG TCCAGCTGCA AGAGTCCGGA CCCGGACTGG TGAAACCTTC CCAGACACTG |
| 781 | TCTCTGACAT GCACAGTGAG CGGATTCTCC CTCACAAGCT ACGGCGTGTA CTGGGTGAGA |
| 841 | CAGCCTCCCG GCAAAGGACT GGAGTGGCTG GGCGTCATCT GGGCTGGAGG CACCACCAAT |
| 901 | TACAACAGCG CTCTGATGTC TAGACTGACA ATCTCCAAGG ACACCAGCAA GAACCAAGTG |
| 961 | TCTCTGAAGC TGAGCTCCGT GACAGCCGCC GATACAGCCG TGTACTATTG TGCCAGAGGC |
| 1021 | CCCCCCCACG CTATGATGAA GAGGGGCTAC GCCATGGACT ACTGGGGCCA AGGCACCCTC |
| 1081 | GTCACAGTGA GCTCCGGAGG CAGCGGAGGA AGCGGCGGCA GCGAGATCGT GCTGACCCAG |
| 1141 | TCCCCCGGCA CACTGTCCCT CAGCCCCGGC GAAAGAGCCA CACTGAGCTG TAGAGCTTCC |
| 1201 | CAGAGCGTGG GCAGCAGCTA TCTGGCTTGG TATCAGCAGA AGCCCGGCCA AGCCCCCAGA |
| 1261 | CTGCTCATCT ACGGAGCCTT CAGCAGAGCC ACCGGCATCC CCGACAGATT CAGCGGCAGC |
| 1321 | GGCAGCGGCA CCGATTTTAC CCTCACCATC TCCAGACTGG AGCCAGAGGA CTTCGCCGTG |
| 1381 | TATTACTGCC AACAGTACGG CAGCAGCCCT TGGACCTTTG GACAAGGCAC CAAGGTGGAA |
| 1441 | ATCAAACACC ACCACCATCA CCATCATCAT CACCAC |

SEQ ID NO: 27 Ipilimumab VH and CH1 amino acid sequence

| | |
|---|---|
| 1 | QVQLVESGGG VVQPGRSLRL SCAASGFTFS SYTMHWVRQA PGKGLEWVTF ISYDGNNKYY |
| 61 | ADSVKGRFTI SRDNSKNTLY LQMNSLRAED TAIYYCARTG WLGPFDYWGQ GTLVTVSSAS |
| 121 | TKGPSVFPLA PSSKSTSGGT AALGCLVKDY FPEPVTVSWN SGALTSGVHT FPAVLQSSGL |
| 181 | YSLSSVVTVP SSSLGTQTYI CNVNHKPSNT KVDKKV |

*SEQ ID NO: 27 optionally comprises the sequence (EPKSCDKT) at the C-terminal end,
which corresponds to the hinge region for F(ab')2 or Fab', where the cysteine residue forms
inter-chain bond with kappa light. Alternatively, F(ab')2 may comprise the sequence (e.g.,
proprietary sequence as indicated in FIG. 10H) that forms inter-chain dimerization domain
C-terminal to SEQ ID NO: 27.
*See Table 3 and FIGS. 10D and 10H.
SEQ ID NO: 28 BioE2023 ipilimumab VL and CL amino acid sequence

| | |
|---|---|
| 1 | EIVLTQSPGT LSLSPGERAT LSCRASQSVG SSYLAWYQQK PGQAPRLLIY GAFSRATGIP |
| 61 | DRFSGSGSGT DFTLTISRLE PEDFAVYYCQ QYGSSPWTFG QGTKVEIKRT VAAPSVFIFP |
| 121 | PSDEQLKSGT ASVVCLLNNF YPREAKVQWK VDNALQSGNS QESVTEQDSK DSTYSLSSTL |
| 181 | TLSKADYEKH KVYACEVTHQ GLSSPVTKSF NRGEC |

SEQ ID NO: 29 BioE2041 (anti-CTLA4 Tandem Biparatopic ScFv (TBS)) amino acid sequence
VL IPI (G4S)3 VH IPI (KEA)6 VL 121 (G4S)3 VH 121 His tag
EIVLTQSPGTLSLSPGERATLSCRASQSVGSSYLAWYQQKPGQAPRLLIYGAFSRATGIPDRESGSGSGTDFT
LTISRLEPEDFAVYYCQQYGSSPWTFGQGTKVEIKGGGGSGGGGSGGGGSQVQLVESGGGVVQPGRSLRLSCA
ASGFTFSSYTMHWVRQAPGKGLEWVTFISYDGNNKYYADSVKGRFTISRDNSKNTLYLQMNSLRAEDTAIYYC
ARTGWLGPFDYWGQGTLVTVSSKEAKEAKEAKEAKEAKEADIQMTQSPSSLSASVGDRVTITCSATSSITYMS
WYQQKPGKAPKLLIYDTSNLASGVPSRFSGSGSGTDYTLTISSLQPEDFATYYCQQWSSYPLTFGGGTKVEIK
GGGGSGGGGSGGGGSQVQLQESGPGLVKPSQTLSLTCTVSGFSLTSYGVYWVRQPPGKGLEWLGVIWAGGTTN
YNSALMSRLTISKDTSKNQVSLKLSSVTAADTAVYYCARGPPHAMMKRGYAMDYWGQGTLVTVSS
*BioE2041 self-dimerizes.
*The underline represents the linker sequence.
SEQ ID NO: 30 BioE2042 (anti-CTLA4 Tandem Biparatopic ScFv (TBS)) amino acid sequence
VL 121 (G4S)3 VH 121 (KEA)6 VL IPI (G4S)3 VH IPI His tag
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKGGGGSGGGGSGGGGSQVQLQESGPGLVKPSQTLSLTCTVS
GFSLTSYGVYWVRQPPGKGLEWLGVIWAGGTTNYNSALMSRLTISKDTSKNQVSLKLSSVTAADTAVYYCARG
PPHAMMKRGYAMDYWGQGTLVTVSSKEAKEAKEAKEAKEAKEAEIVLTQSPGTLSLSPGERATLSCRASQSVG
SSYLAWYQQKPGQAPRLLIYGAFSRATGIPDRESGSGSGTDFTLTISRLEPEDFAVYYCQQYGSSPWTFGQGT
KVEIKGGGGSGGGGSGGGGSQVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFDYWGQGTLVTVSS
*BioE2042 self-dimerizes.
*The underline represents the linker sequence.
BioE2061 (anti-CTLA4 Tandem Fab) amino acid sequence
VH121-CH-(G4S)3-VH IPI-CH-His tag (VL 121-CL/VL IPI-CL)
BioE2061 comprises 3 polypeptides with the sequence of SEQ ID Nos: 31-33. See Wu et
al. (2015) Mabs 7: 470-82
SEQ ID NO: 31
121-IPI Fab HC
QVQLQESGPGLVKPSQTLSLTCTVSGFSLTSYGVYWVRQPPGKGLEWLGVIWAGGTTNYNSALMSRLTISKDT
SKNQVSLKLSSVTAADTAVYYCARGPPHAMMKRGYAMDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDK
KVEPKSCDKTGGGGSGGGGSGGGGSQVQLVESGGGVVQPGRSLRLSCAASGFTFSSYTMHWVRQAPGKGLEWV
TFISYDGNNKYYADSVKGRFTISRDNSKNTLYLQMNSLRAEDTAIYYCARTGWLGPFDYWGQGTLVTVSSAST
KGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHHHHHHHHHH
SEQ ID NO: 32
121 LC
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
SEQ ID NO: 33
IPI LC
EIVLTQSPGTLSLSPGERATLSCRASQSVGSSYLAWYQQKPGQAPRLLIYGAFSRATGIPDRESGSGSGTDET
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
BioE2062 (anti-CTLA4 Tandem Fab) amino acid sequence
VH IPI-CH-(G4S)3-VH 121-CH-His tag (VL IPI-CL/VL 121-CL)
BioE2062 comprises 3 polypeptides with the sequence of SEQ ID Nos: 34-36. See Wu et
al. (2015) Mabs 7: 470-82
SEQ ID NO: 34
IPI-121 Fab HC
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTGGGGSGGGGSGGGGSQVQLQESGPGLVKPSQTLSLTCTVSGFSLTSYGVYWVRQPPGKGLEWLGVIWA
GGTTNYNSALMSRLTISKDTSKNQVSLKLSSVTAADTAVYYCARGPPHAMMKRGYAMDYWGQGTLVTVSSAST
KGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKT
SEQ ID NO: 35
IPI LC
EIVLTQSPGTLSLSPGERATLSCRASQSVGSSYLAWYQQKPGQAPRLLIYGAFSRATGIPDRFSGSGSGTDET
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 36
121 LC
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
BioE2091 (anti-CTLA4 Tandem Fab) amino acid sequence
VL IPI CL (G4S)3 VH 121 CH1 His tag (VH IPI CH1/VL 121 CL)
BioE2091 comprises 3 polypeptides with the sequence of SEQ ID Nos: 37-39. See Wu et
al. (2015) Mabs 7: 470-82
SEQ ID NO: 37
IPI LC-121 HC Fab
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGECGGGG
SGGGGSGGGGSQVQLQESGPGLVKPSQTLSLTCTVSGFSLTSYGVYWVRQPPGKGLEWLGVIWAGGTTNYNSA
LMSRLTISKDTSKNQVSLKLSSVTAADTAVYYCARGPPHAMMKRGYAMDYWGQGTLVTVSSASTKGPSVFPLA
PSSKSTSGGTAALGCLVKDYFPEPVTVSWNSGALTSGVHTFPAVLQSSGLYSLSSVVTVPSSSLGTQTYICNV
NHKPSNTKVDKKVEPKSCDKT
SEQ ID NO: 38
IPI HC Fab
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKT
SEQ ID NO: 39
121 LC
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
BioE2092 (anti-CTLA4 Tandem Fab) amino acid sequence
VL 121 CL (G4S)3 VH IPI CHI His tag (VH 121 CH1/VL IPI CL)
BioE2092 comprises 3 polypeptides with the sequence of SEQ ID Nos: 40-42. See Wu et
al. (2015) Mabs 7: 470-82
SEQ ID NO: 40
121 LC-IPI HC Fab
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGECGGGGSG
GGGSGGGGSQVQLVESGGGVVQPGRSLRLSCAASGFTFSSYTMHWVRQAPGKGLEWVTFISYDGNNKYYADSV
KGRFTISRDNSKNTLYLQMNSLRAEDTAIYYCARTGWLGPFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNT
KVDKKVEPKSCDKT
SEQ ID NO: 41
121 HC Fab
QVQLQESGPGLVKPSQTLSLTCTVSGFSLTSYGVYWVRQPPGKGLEWLGVIWAGGTTNYNSALMSRLTISKDT
SKNQVSLKLSSVTAADTAVYYCARGPPHAMMKRGYAMDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDK
KVEPKSCDKT
SEQ ID NO: 42
IPI LC
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
BioE2111 (anti-CTLA4 DART) amino acid sequence
BioE2111 comprises 2 polypeptides with the sequence of SEQ ID Nos: 43-44.
SEQ ID NO: 43
VL IPI G3SG4 VH121GGCGGGEVAALEKEVAALEKEVAALEKEVAALEK
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKGGGSGGGGQVQLQESGPGLVKPSQTLSLTCTVSGFSLT
SYGVYWVRQPPGKGLEWLGVIWAGGTTNYNSALMSRLTISKDTSKNQVSLKLSSVTAADTAVYYCARGPPHAM
MKRGYAMDYWGQGTLVTVSSGGCGGGEVAALEKEVAALEKEVAALEKEVAALEK
*The underline represents the linker sequence.
SEQ ID NO: 44
VL121 G3SG4 VH IPI GGCGGGKVAALKEKVAALKEKVAALKEKVAALKE
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKGGGSGGGGQVQLVESGGGVVQPGRSLRLSCAASGFTFSSY
TMHWVRQAPGKGLEWVTFISYDGNNKYYADSVKGRFTISRDNSKNTLYLQMNSLRAEDTAIYYCARTGWLGPF
DYWGQGTLVTVSSGGCGGGKVAALKEKVAALKEKVAALKEKVAALKE
*The underline represents the linker sequence.
BioE2012 (anti-CTLA4 Half antibody-scFv)
BioE2012 comprises 2 polypeptides with the sequence of SEQ ID Nos: 45-46.
IPI Fab Fc 121 ScFv [VL121 (G4S)3 VH 121]
*BioE2012 contains the Fc-null LALAPG mutation
SEQ ID NO: 45
IPI 1/2Ab-121 scFv
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTSPPSPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAKGQPREPQVYTLPPSRDELTKNQVS
LTCLVKGFYPSDIAVEWESNGQPENNYKTTPPVLDSDGSFRLYSRLTVDKSRWQQGNVFSCSVMHEALHNHYT
QKSLSLSPGGGGGSGGGGSDIQMTQSPSSLSASVGDRVTITCSATSSITYMSWYQQKPGKAPKLLIYDTSNLA
SGVPSRFSGSGSGTDYTLTISSLQPEDFATYYCQQWSSYPLTFGGGTKVEIKGGGGSGGGGSGGGGSQVQLQE
SGPGLVKPSQTLSLTCTVSGFSLTSYGVYWVRQPPGKGLEWLGVIWAGGTTNYNSALMSRLTISKDTSKNQVS
LKLSSVTAADTAVYYCARGPPHAMMKRGYAMDYWGQGTLVTVSS
SEQ ID NO: 46
IPI LC
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
BioE2081 (anti-CTLA4 Heterodimeric Fab/scFv-Fc)
BioE2081 comprises 3 polypeptides with the sequence of SEQ ID Nos: 47-49.
121ScFv Fc IPI Fab Fc (Fc silent) KiH Fc
*KiH stands for Knob in Hole. See e.g., Ridgway et al. (1996) Protein Engineering 9:
617-621
*BioE2081 contains the Fc-silent LALAPG mutation
SEQ ID NO: 47
IPI Fab-knob Fc
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTCPPCPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAKGQPREPQVYTLPPCRDELTKNQVS
LWCLVKGFYPSDIAVEWESNGQPENNYKTTPPVLDSDGSFFLYSKLTVDKSRWQQGNVFSCSVMHEALHNHYT
QKSLSLSPG
SEQ ID NO: 48
121 scFv-hole Fc
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKGGGGSGGGGSGGGGSQVQLQESGPGLVKPSQTLSLTCTVS
GFSLTSYGVYWVRQPPGKGLEWLGVIWAGGTTNYNSALMSRLTISKDTSKNQVSLKLSSVTAADTAVYYCARG
PPHAMMKRGYAMDYWGQGTLVTVSSGGGGSEPKSQDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALGAP
IEKTISKAKGQPREPQVCTLPPSRDELTKNQVSLSCAVKGFYPSDIAVEWESNGQPENNYKTTPPVLDSDGSF
FLVSKLTVDKSRWQQGNVFSCSVMHEALHNHYTQKSLSLSPGK
SEQ ID NO: 49
IPI LC
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
BioE2082 (anti-CTLA4 Heterodimeric Fab/scFv-Fc)
BioE2082 comprises 3 polypeptides with the sequence of SEQ ID Nos: 50-52.
121 Fab Fc IPI ScFv Fc (Fc silent) KiH Fc
*KiH stands for Knob in Hole. See e.g., Ridgway et al. (1996) Protein Engineering 9:
617-621
*BioE2082 contains the Fc-silent LALAPG mutation
SEQ ID NO: 50
121 Fab-knob Fc
QVQLQESGPGLVKPSQTLSLTCTVSGFSLTSYGVYWVRQPPGKGLEWLGVIWAGGTTNYNSALMSRLTISKDT
SKNQVSLKLSSVTAADTAVYYCARGPPHAMMKRGYAMDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALGAPIEKTISKAKGQPREPQVYTLPPCRDELT
KNQVSLWCLVKGFYPSDIAVEWESNGQPENNYKTTPPVLDSDGSFFLYSKLTVDKSRWQQGNVFSCSVMHEAL
HNHYTQKSLSLSPG
SEQ ID NO: 51
IPI scFv-hole Fc
EIVLTQSPGTLSLSPGERATLSCRASQSVGSSYLAWYQQKPGQAPRLLIYGAFSRATGIPDRESGSGSGTDFT
LTISRLEPEDFAVYYCQQYGSSPWTFGQGTKVEIKGGGGSGGGGSGGGGSQVQLVESGGGVVQPGRSLRLSCA
ASGFTFSSYTMHWVRQAPGKGLEWVTFISYDGNNKYYADSVKGRFTISRDNSKNTLYLQMNSLRAEDTAIYYC
ARTGWLGPFDYWGQGTLVTVSSGGGGSEPKSQDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALGAPIEK
TISKAKGQPREPQVCTLPPSRDELTKNQVSLSCAVKGFYPSDIAVEWESNGQPENNYKTTPPVLDSDGSFFLV
SKLTVDKSRWQQGNVFSCSVMHEALHNHYTQKSLSLSPGK
SEQ ID NO: 52
121 LC
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
BioE2121 (anti-CTLA4 Heterodimeric scFvs)
BioE2121 comprises 2 polypeptides with the sequence of SEQ ID Nos: 53-54.
IPI scFv-Fc / 121 scFv-Fc (KiH)
*KiH stands for Knob in Hole. See e.g., Ridgway et al. (1996) Protein Engineering 9:
617-621
*BioE2121 contains the Fc-silent LALAPG mutation
SEQ ID NO: 53
IPI scFv-knob Fc
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKGGGGSGGGGSGGGGSQVQLVESGGGVVQPGRSLRLSCA
ASGFTFSSYTMHWVRQAPGKGLEWVTFISYDGNNKYYADSVKGRFTISRDNSKNTLYLQMNSLRAEDTAIYYC
ARTGWLGPFDYWGQGTLVTVSSGGGGSEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALGAPIEK
TISKAKGQPREPQVYTLPPCRDELTKNQVSLWCLVKGFYPSDIAVEWESNGQPENNYKTTPPVLDSDGSFFLY
SKLTVDKSRWQQGNVFSCSVMHEALHNHYTQKSLSLSPG
SEQ ID NO: 54
121 scFv-hole Fc
DIQMTQSPSSLSASVGDRVTITCSATSSITYMSWYQQKPGKAPKLLIYDTSNLASGVPSRFSGSGSGTDYTLT
ISSLQPEDFATYYCQQWSSYPLTFGGGTKVEIKGGGGSGGGGSGGGGSQVQLQESGPGLVKPSQTLSLTCTVS
GFSLTSYGVYWVRQPPGKGLEWLGVIWAGGTTNYNSALMSRLTISKDTSKNQVSLKLSSVTAADTAVYYCARG
PPHAMMKRGYAMDYWGQGTLVTVSSGGGGSEPKSQDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALGAP
IEKTISKAKGQPREPQVCTLPPSRDELTKNQVSLSCAVKGFYPSDIAVEWESNGQPENNYKTTPPVLDSDGSF
FLVSKLTVDKSRWQQGNVFSCSVMHEALHNHYTQKSLSLSPGK
SEQ ID NO: 55
An exemplary leader sequence that may be included in the antigen-binding protein of the
present disclosure.
MDFQVQIFSFLLISASVILSRG
SEQ ID NO: 56
BioE2410 amino acid sequence
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSGGSGGSGGSDIQMTQSPSSLSASVGDRV
TITCSATSSITYMSWYQQKPGKAPKLLIYDTSNLASGVPSRFSGSGSGTDYTLTISSLQPEDFATYYCQQWSS
YPLTFGGGTKVEIKGGSGGSGGSQVQLQESGPGLVKPSQTLSLTCTVSGFSLTSYGVYWVRQPPGKGLEWLGV
IWAGGTTNYNSALMSRLTISKDTSKNQVSLKLSSVTAADTAVYYCARGPPHAMMKRGYAMDYWGQGTLVTVSS
GGSGGSGGSEIVLTQSPGTLSLSPGERATLSCRASQSVGSSYLAWYQQKPGQAPRLLIYGAFSRATGIPDRFS
GSGSGTDFTLTISRLEPEDFAVYYCQQYGSSPWTFGQGTKVEIKGGSGGDKTHTCPPCPAPELLGGPSVFLFP
PKPKDTLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 57
BioE2410 nucleic acid sequence
CAAGTGCAGCTGGTGGAGAGCGGTGGCGGCGTGGTGCAGCCCGGTAGAAGCCTGAGACTGAGCTGCGCCGCAA
GCGGCTTCACCTTCAGCAGCTACACCATGCACTGGGTGAGACAAGCACCCGGCAAGGGACTGGAATGGGTCAC
GTTCATCAGCTACGACGGCAACAACAAGTACTACGCCGACAGCGTGAAGGGCAGATTCACCATCAGCCGAGAC
AACAGCAAGAACACCCTGTACCTGCAGATGAACAGCCTGAGAGCCGAGGACACCGCCATCTACTATTGCGCTA
GAACGGGATGGCTGGGCCCCTTCGATTATTGGGGGCAAGGCACGTTGGTGACCGTCTCAAGCGGCGGAAGCGG
AGGCAGCGGAGGATCGGATATACAGATGACACAGTCGCCTAGCTCACTGAGCGCAAGCGTGGGCGACAGAGTG
ACCATCACCTGCAGCGCCACAAGCAGCATCACCTACATGAGCTGGTATCAGCAAAAACCCGGCAAGGCCCCCA
AGCTGCTGATCTACGATACAAGCAACCTGGCAAGCGGCGTGCCTAGCAGATTCTCTGGCAGCGGCAGCGGCAC
CGACTATACTCTCACCATAAGCAGTCTACAGCCTGAGGATTTCGCCACCTACTATTGTCAGCAGTGGTCGAGC
TACCCCCTGACCTTCGGGGGCGGCACCAAGGTAGAGATTAAAGGTGGGAGCGGCGGGAGTGGTGGCAGCCAAG
TCCAACTGCAAGAGTCCGGACCCGGTCTTGTGAAGCCTTCTCAGACTCTTAGCTTAACATGCACCGTGAGCGG
CTTCAGCCTTACAAGCTACGGCGTCTACTGGGTACGTCAACCACCTGGTAAGGGTTTAGAATGGCTAGGGGTG
ATCTGGGCCGGCGGCACCACCAACTACAACAGCGCCCTGATGAGCAGACTGACGATCTCTAAGGACACTAGCA
AAAACCAAGTGAGCCTCAAGCTGAGCAGCGTGACCGCCGCCGACACCGCCGTATACTACTGTGCAAGAGGCCC
CCCCCACGCCATGATGAAGAGAGGCTACGCCATGGATTACTGGGGGCAAGGCACTCTAGTGACCGTGAGTAGT
GGCGGGAGCGGTGGGAGCGGCGGTTCCGAAATCGTGCTGACACAGAGCCCTGGCACTCTGTCTCTGAGTCCCG
GCGAGAGAGCTACCCTGAGCTGCAGAGCATCTCAGAGCGTGGGCAGCAGCTACCTGGCCTGGTATCAGCAAAA
GCCCGGCCAAGCCCCTAGACTTCTGATCTACGGCGCATTCAGCAGAGCCACCGGCATCCCCGACAGATTCAGT
GGATCTGGCAGCGGAACGGACTTCACCCTAACAATCAGCCGTCTGGAACCCGAAGACTTTGCGGTGTATTACT
GTCAACAGTACGGTAGCAGCCCCTGGACCTTCGGCCAAGGCACAAAGGTGGAGATCAAAGGCGGTTCCGGTGG
CGACAAAACACACACCTGTCCCCCCTGCCCCGCCCCTGAGCTGTTAGGTGGTCCTAGCGTGTTCCTGTTCCCC
CCCAAGCCCAAGGACACCCTGATGATCAGCAGAACCCCTGAGGTGACCTGCGTGGTGGTGGACGTGAGCCACG
AGGACCCTGAGGTGAAGTTCAACTGGTACGTGGACGGCGTGGAGGTGCACAACGCCAAGACCAAGCCTAGAGA
GGAGCAGTACAACAGCACCTACAGAGTGGTGAGCGTGCTGACCGTGCTGCACCAAGACTGGCTGAACGGCAAG
GAGTACAAGTGCAAGGTGAGCAACAAGGCCCTGCCCGCCCCCATCGAGAAGACCATCAGCAAGGCCAAGGGGC
AGCCTAGAGAGCCCCAAGTGTACACCCTGCCCCCTAGCAGAGACGAGCTGACCAAGAACCAAGTGTCGCTAAC
CTGTTTGGTGAAGGGCTTCTACCCTAGCGACATCGCCGTGGAGTGGGAGAGCAACGGGCAGCCTGAGAACAAC
TACAAGACCACCCCCCCCGTGCTGGACAGCGACGGCAGCTTCTTCCTGTACAGCAAGCTGACCGTGGACAAGA
GCAGATGGCAGCAAGGCAACGTGTTCAGCTGCAGCGTGATGCACGAGGCCCTGCACAACCACTACACACAGAA
GAGCCTGTCGCTGAGCCCCGGAAAA
SEQ ID NO: 58
BioE2420 amino acid sequence
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSGGSGGSGGSDIQMTQSPSSLSASVGDRV
TITCSATSSITYMSWYQQKPGKAPKLLIYDTSNLASGVPSRFSGSGSGTDYTLTISSLQPEDFATYYCQQWSS
YPLTFGGGTKVEIKGGSGGSGGSQVQLQESGPGLVKPSQTLSLTCTVSGFSLTSYGVYWVRQPPGKGLEWLGV
IWAGGTTNYNSALMSRLTISKDTSKNQVSLKLSSVTAADTAVYYCARGPPHAMMKRGYAMDYWGQGTLVTVSS
GGSGGSGGSEIVLTQSPGTLSLSPGERATLSCRASQSVGSSYLAWYQQKPGQAPRLLIYGAFSRATGIPDRES
GSGSGTDFTLTISRLEPEDFAVYYCQQYGSSPWTFGQGTKVEIKGGSGGDKTHTCPPCPAPEAAGGPSVFLFP
PKPKDTLMISRTPEVTCVVVDVSHEDPEVKFNWYVDGVEVHNAKTKPREEQYNSTYRVVSVLTVLHQDWLNGK
EYKCKVSNKALGAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 59
BioE2420 nucleic acid sequence
CAAGTGCAGCTGGTGGAGAGCGGTGGCGGCGTGGTGCAGCCCGGTAGAAGCCTGAGACTGAGCTGCGCCGCAA
GCGGCTTCACCTTCAGCAGCTACACCATGCACTGGGTGAGACAAGCACCCGGCAAGGGACTGGAATGGGTCAC
GTTCATCAGCTACGACGGCAACAACAAGTACTACGCCGACAGCGTGAAGGGCAGATTCACCATCAGCCGAGAC
AACAGCAAGAACACCCTGTACCTGCAGATGAACAGCCTGAGAGCCGAGGACACCGCCATCTACTATTGCGCTA
GAACGGGATGGCTGGGCCCCTTCGATTATTGGGGGCAAGGCACGTTGGTGACCGTCTCAAGCGGCGGAAGCGG
AGGCAGCGGAGGATCGGATATACAGATGACACAGTCGCCTAGCTCACTGAGCGCAAGCGTGGGCGACAGAGTG
ACCATCACCTGCAGCGCCACAAGCAGCATCACCTACATGAGCTGGTATCAGCAAAAACCCGGCAAGGCCCCCA
AGCTGCTGATCTACGATACAAGCAACCTGGCAAGCGGCGTGCCTAGCAGATTCTCTGGCAGCGGCAGCGGCAC
CGACTATACTCTCACCATAAGCAGTCTACAGCCTGAGGATTTCGCCACCTACTATTGTCAGCAGTGGTCGAGC
TACCCCCTGACCTTCGGGGGCGGCACCAAGGTAGAGATTAAAGGTGGGAGCGGCGGGAGTGGTGGCAGCCAAG
TCCAACTGCAAGAGTCCGGACCCGGTCTTGTGAAGCCTTCTCAGACTCTTAGCTTAACATGCACCGTGAGCGG
CTTCAGCCTTACAAGCTACGGCGTCTACTGGGTACGTCAACCACCTGGTAAGGGTTTAGAATGGCTAGGGGTG
ATCTGGGCCGGCGGCACCACCAACTACAACAGCGCCCTGATGAGCAGACTGACGATCTCTAAGGACACTAGCA
AAAACCAAGTGAGCCTCAAGCTGAGCAGCGTGACCGCCGCCGACACCGCCGTATACTACTGTGCAAGAGGCCC
CCCCCACGCCATGATGAAGAGAGGCTACGCCATGGATTACTGGGGGCAAGGCACTCTAGTGACCGTGAGTAGT
GGCGGGAGCGGTGGGAGCGGCGGTTCCGAAATCGTGCTGACACAGAGCCCTGGCACTCTGTCTCTGAGTCCCG
GCGAGAGAGCTACCCTGAGCTGCAGAGCATCTCAGAGCGTGGGCAGCAGCTACCTGGCCTGGTATCAGCAAAA
GCCCGGCCAAGCCCCTAGACTTCTGATCTACGGCGCATTCAGCAGAGCCACCGGCATCCCCGACAGATTCAGT
GGATCTGGCAGCGGAACGGACTTCACCCTAACAATCAGCCGTCTGGAACCCGAAGACTTTGCGGTGTATTACT
GTCAACAGTACGGTAGCAGCCCCTGGACCTTCGGCCAAGGCACAAAGGTGGAGATCAAAGGCGGTTCCGGTGG
CGACAAAACACACACCTGTCCCCCCTGCCCCGCCCCTGAGGCTGCCGGTGGTCCTAGCGTGTTCCTGTTCCCC
CCCAAGCCCAAGGACACCCTGATGATCAGCAGAACCCCTGAGGTGACCTGCGTGGTGGTGGACGTGAGCCACG
AGGACCCTGAGGTGAAGTTCAACTGGTACGTGGACGGCGTGGAGGTGCACAACGCCAAGACCAAGCCTAGAGA
GGAGCAGTACAACAGCACCTACAGAGTGGTGAGCGTGCTGACCGTGCTGCACCAAGACTGGCTGAACGGCAAG
GAGTACAAGTGCAAGGTGAGCAACAAGGCCCTGGGCGCCCCCATCGAGAAGACCATCAGCAAGGCCAAGGGGC
AGCCTAGAGAGCCCCAAGTGTACACCCTGCCCCCTAGCAGAGACGAGCTGACCAAGAACCAAGTGTCGCTAAC
CTGTTTGGTGAAGGGCTTCTACCCTAGCGACATCGCCGTGGAGTGGGAGAGCAACGGGCAGCCTGAGAACAAC
TACAAGACCACCCCCCCCGTGCTGGACAGCGACGGCAGCTTCTTCCTGTACAGCAAGCTGACCGTGGACAAGA
GCAGATGGCAGCAAGGCAACGTGTTCAGCTGCAGCGTGATGCACGAGGCCCTGCACAACCACTACACACAGAA
GAGCCTGTCGCTGAGCCCCGGAAAA
SEQ ID NO: 60
BioE2430 amino acid sequence
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSGGSGGSGGSDIQMTQSPSSLSASVGDRV
TITCSATSSITYMSWYQQKPGKAPKLLIYDTSNLASGVPSRFSGSGSGTDYTLTISSLQPEDFATYYCQQWSS
YPLTFGGGTKVEIKGGSGGSGGSQVQLQESGPGLVKPSQTLSLTCTVSGFSLTSYGVYWVRQPPGKGLEWLGV
IWAGGTTNYNSALMSRLTISKDTSKNQVSLKLSSVTAADTAVYYCARGPPHAMMKRGYAMDYWGQGTLVTVSS
GGSGGSGGSEIVLTQSPGTLSLSPGERATLSCRASQSVGSSYLAWYQQKPGQAPRLLIYGAFSRATGIPDRFS
GSGSGTDFTLTISRLEPEDFAVYYCQQYGSSPWTFGQGTKVEIKGGSGGEAHKSEIAHRYNDLGEQHFKGLVL
IAFSQYLQKCSYDEHAKLVQEVTDFAKTCVADESAANCDKSLHTLFGDKLCAIPNLRENYGELADCCTKQEPE
RNECFLQHKDDNPSLPPFERPEAEAMCTSFKENPTTEMGHYLHEVARRHPYFYAPELLYYAEQYNEILTQCCA
EADKESCLTPKLDGVKEKALVSSVRQRMKCSSMQKFGERAFKAWAVARLSQTFPNADFAEITKLATDLTKVNK
ECCHGDLLECADDRAELAKYMCENQATISSKLQTCCDKPLLKKAHCLSEVEHDTMPADLPAIAADFVEDQEVC
KNYAEAKDVFLGTFLYEYSRRHPDYSVSLLLRLAKKYEATLEKCCAEANPPACYGTVLAEFQPLVEEPKNLVK
TNCDLYEKLGEYGFQNAILVRYTQKAPQVSTPTLVEAARNLGRVGTKCCTLPEDQRLPCVEDYLSAILNRVCL
LHEKTPVSEHVTKCCSGSLVERRPCFSALTVDETYVPKEFKAETFTFHSDICTLPEKEKQIKKQTALAELVKH
KPKATAEQLKTVMDDFAQFLDTCCKAADKDTCFSTEGPNLVTRCKDALAHHHHHHHHHH
SEQ ID NO: 61
BioE2430 nucleic acid sequence
CAAGTGCAATTGGTAGAGTCCGGCGGCGGCGTTGTGCAACCCGGCAGAAGCCTTAGACTGAGCTGCGCCGCAA
GCGGCTTCACCTTCAGCAGCTACACCATGCACTGGGTGAGACAAGCCCCCGGCAAGGGCCTGGAATGGGTGAC
CTTCATCAGCTACGACGGCAACAACAAGTACTACGCTGATAGCGTGAAGGGCAGATTCACTATCAGCCGTGAC
AACAGCAAGAACACCCTGTACCTGCAGATGAACAGCCTGAGAGCCGAGGACACCGCCATCTACTACTGCGCAA
GAACCGGCTGGCTGGGCCCCTTCGACTACTGGGGCCAAGGGACATTAGTTACTGTTAGCAGCGGCGGGAGCGG
GGGCAGCGGAGGATCGGATATACAGATGACACAGAGCCCTAGCAGCCTGAGCGCAAGCGTGGGCGACAGAGTG
ACCATCACCTGCAGCGCCACAAGCAGCATCACCTACATGAGCTGGTATCAGCAAAAACCCGGCAAGGCCCCCA
AGCTGCTGATCTACGACACAAGCAACCTGGCAAGCGGCGTGCCTAGCAGATTCTCTGGCAGCGGCAGCGGCAC
AGATTATACTCTCACAATCAGCAGTCTGCAGCCTGAGGATTTCGCCACATACTACTGCCAACAGTGGAGCAGC
TACCCCCTGACCTTCGGCGGCGGCACTAAAGTAGAGATTAAAGGCGGCAGCGGTGGAAGTGGCGGGAGCCAAG
TGCAACTGCAAGAGAGCGGTCCCGGCCTGGTGAAGCCTTCTCAGACCTTGAGCCTTACCTGCACCGTGAGCGG
CTTCAGCCTGACAAGCTACGGCGTGTACTGGGTGCGGCAACCCCCCGGCAAAGGCTTGGAATGGCTGGGGGTG
ATCTGGGCCGGCGGCACCACCAACTACAACAGCGCCCTGATGAGCAGACTGACCATTAGCAAGGACACAAGCA
AGAACCAAGTGAGCCTGAAGCTATCTAGCGTGACCGCCGCCGACACCGCCGTCTATTACTGTGCAAGAGGCCC
CCCCCACGCCATGATGAAGAGAGGCTACGCCATGGATTATTGGGGCCAAGGCACCCTGGTTACCGTCTCTTCC
GGGGGTAGCGGCGGCAGCGGAGGTTCCGAGATCGTGTTAACTCAGAGCCCTGGCACCCTGTCGCTGAGCCCTG
GTGAAAGGGCTACGCTAAGCTGCAGAGCATCTCAGAGCGTGGGCAGCAGCTACCTGGCCTGGTATCAGCAGAA
ACCCGGCCAAGCCCCTAGACTGCTGATCTATGGTGCCTTCAGCAGAGCCACCGGCATCCCCGACAGATTCAGC
GGTTCCGGTAGTGGCACAGACTTCACTCTGACCATCAGCCGGCTAGAACCTGAGGATTTTGCCGTTTACTACT
GTCAGCAGTATGGCAGCAGCCCCTGGACCTTCGGCCAAGGCACGAAGGTGGAGATAAAAGGTGGAAGCGGCGG
GGAAGCCCACAAGAGCGAGATCGCCCACAGATACAACGACCTGGGCGAGCAGCACTTCAAGGGCCTGGTGCTG
ATCGCCTTCTCTCAGTACCTGCAGAAGTGCAGCTACGACGAGCACGCCAAGCTGGTGCAAGAGGTGACCGACT
TCGCCAAGACCTGCGTGGCCGACGAGAGCGCCGCCAACTGCGACAAGAGCCTGCACACCCTGTTCGGCGACAA
GCTGTGCGCCATCCCCAACCTGAGAGAGAACTACGGCGAGCTGGCCGACTGCTGCACCAAGCAAGAGCCGGAG
AGAAATGAGTGCTTCCTGCAGCACAAGGACGACAACCCTAGCCTGCCCCCCTTCGAGAGACCTGAGGCCGAGG
CCATGTGCACTAGCTTCAAGGAGAACCCCACCACCTTCATGGGCCACTACCTGCACGAGGTGGCTAGACGTCA
TCCATATTTCTACGCCCCTGAGCTGCTGTACTATGCAGAGCAGTACAACGAGATCCTGACCCAATGTTGTGCC
GAGGCGGACAAAGAGAGCTGCCTGACCCCCAAGCTGGACGGCGTGAAGGAGAAGGCCCTGGTGAGCAGCGTCA
GACAGAGAATGAAGTGCAGCAGCATGCAGAAGTTCGGGGAGAGAGCTTTCAAGGCCTGGGCCGTGGCTAGACT
GTCTCAGACCTTCCCCAACGCCGACTTCGCCGAGATCACCAAGCTGGCCACCGACCTGACCAAGGTGAACAAG
GAGTGCTGCCACGGCGACCTGCTGGAGTGCGCCGACGACAGAGCCGAGCTGGCCAAGTACATGTGCGAGAACC
AAGCCACTATCTCATCCAAATTACAGACCTGCTGCGACAAGCCCCTGCTGAAGAAAGCTCATTGCCTCAGCGA
GGTGGAGCATGACACCATGCCCGCCGATCTGCCCGCCATCGCCGCCGACTTCGTGGAGGACCAAGAGGTGTGC
AAGAACTACGCCGAGGCGAAGGATGTGTTCCTGGGCACCTTCCTGTACGAGTACAGCCGAAGGCACCCCGACT
ACAGCGTGAGCCTGCTGCTGAGACTGGCCAAGAAGTACGAGGCCACTTTGGAGAAATGCTGCGCTGAGGCAAA
CCCCCCTGCCTGCTACGGCACCGTGCTGGCCGAGTTTCAGCCCCTGGTAGAGGAGCCGAAGAACCTGGTGAAG
ACCAACTGCGACCTGTACGAGAAGCTGGGCGAGTACGGCTTTCAGAACGCCATCCTGGTGAGATACACACAGA
AGGCCCCCCAAGTGAGCACCCCCACCTTGGTGGAAGCCGCAAGAAACCTGGGCAGAGTGGGCACCAAATGTTG
CACGCTGCCGGAGGACCAAAGACTGCCCTGCGTGGAGGACTACCTGAGCGCCATCCTGAACAGAGTGTGCCTG
CTGCACGAGAAGACCCCCGTGAGCGAGCACGTGACCAAGTGCTGCAGTGGCAGCCTGGTGGAGAGAAGACCCT
GCTTCAGCGCCCTGACCGTGGACGAGACCTACGTGCCCAAGGAGTTCAAGGCCGAGACCTTCACCTTCCACAG
CGACATCTGTACTCTCCCTGAGAAGGAGAAGCAGATCAAGAAGCAGACCGCCCTGGCCGAGCTGGTGAAGCAC
AAGCCCAAGGCCACCGCCGAGCAGCTGAAGACCGTGATGGACGACTTCGCTCAGTTCCTGGACACCTGCTGCA
AGGCCGCCGACAAGGACACCTGCTTCAGCACCGAGGGCCCCAACCTGGTGACAAGATGCAAGGACGCCCTGGC
CCACCACCACCACCACCACCACCACCACCAC
SEQ ID NO: 62
BioE2440 VH amino acid sequence
DVQLQESGPGLVKPSQSLSLTCSVTGYSITSGYVWNWIRQFPGNKLEWMGYISHDGNTNYNPSLKNRISITRD
TSKNQFFLKLNSVTTEDSATYYCTRNYGYGGTMDYWGQGTAVSVSSAKTTAPSVYPLAPVCGDTTGSSVTLGC
LVKGYFPEPVTLTWNSGSLSSGVHTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVSEDDPDVQISWFVNNVEVHTAQTQ
THREDYNSTLRVVSALPIQHQDWMSGKEFKCKVNNKDLGAPIERTISKPKGSVRAPQVYVLPPPEEEMTKKQV
TLTCMVTDFMPEDIYVEWTNNGKTELNYKNTEPVLDSDGSYFMYSKLRVEKKNWVERNSYSCSVVHEGLHNHH
TTKSFSRTPGK
SEQ ID NO: 63
BioE2440 VH nucleic acid sequence
GACGTGCAGCTGCAAGAGAGCGGCCCCGGCCTGGTGAAGCCGAGCCAATCTCTGAGCCTGACCTGCAGTGTGA
CGGGATACTCAATCACCTCTGGTTACGTGTGGAACTGGATCAGACAGTTCCCCGGCAACAAGCTGGAGTGGAT
GGGCTACATCAGCCACGACGGCAACACCAACTACAACCCTAGCCTGAAGAACAGAATCAGCATAACAAGAGAC
ACAAGCAAGAATCAGTTCTTCCTGAAGCTGAACAGCGTGACCACCGAGGACAGCGCCACCTACTACTGCACAA
GAAACTACGGCTACGGCGGCACCATGGACTACTGGGGCCAAGGCACCGCCGTGAGCGTAAGCAGCGCCAAGAC
AACAGCCCCTAGCGTGTACCCGCTGGCCCCCGTTTGCGGCGACACCACCGGTAGCAGCGTGACCCTGGGGTGC
CTGGTGAAGGGCTACTTCCCTGAGCCCGTGACCCTGACTTGGAATAGCGGCAGCCTGAGCAGCGGCGTGCACA
CCTTCCCCGCCGTGCTGCAGAGCGACCTGTACACCCTGAGCAGCAGCGTGACAGTGACGAGCAGCACCTGGCC
TAGCCAAAGTATCACCTGCAACGTGGCCCACCCCGCAAGCAGCACCAAGGTGGACAAGAAGATCGAACCAAGA
GGCCCCACTATTAAACCTTGCCCCCCCTGCAAGTGTCCCGCACCCAATGCAGCAGGCGGCCCTAGCGTGTTCA
TCTTCCCCCCCAAGATCAAGGACGTGCTGATGATCAGCCTGAGCCCCATCGTGACCTGCGTGGTGGTGGACGT
GAGCGAGGACGACCCCGACGTGCAGATCAGCTGGTTCGTGAACAACGTGGAGGTGCACACCGCTCAGACACAG
ACCCACAGAGAGGACTACAACAGCACCCTGAGAGTGGTGAGCGCCCTGCCCATTCAGCACCAAGACTGGATGA
GCGGCAAGGAGTTCAAGTGCAAGGTGAACAACAAGGACCTGGGCGCCCCCATCGAGAGAACCATCAGCAAGCC
CAAGGGCAGCGTGAGAGCCCCCCAAGTGTACGTGCTGCCCCCCCCTGAGGAGGAGATGACCAAGAAGCAAGTG
ACCCTAACCTGTATGGTGACCGACTTCATGCCTGAGGACATCTACGTGGAGTGGACCAACAACGGCAAGACCG
AGCTGAACTACAAGAACACCGAGCCCGTGCTGGACAGCGACGGCAGCTACTTCATGTACAGCAAGCTGAGAGT
GGAGAAGAAGAACTGGGTGGAGAGAAACAGCTACAGCTGCAGCGTGGTGCACGAGGGCCTGCACAACCACCAC
ACCACCAAGAGCTTCAGCAGAACCCCCGGCAAG
SEQ ID NO: 64
BioE2440 VL amino acid sequence
DIVMTQSQKFMSTSVGDRVSVTCKASQNVGTYVAWYEQKLGQSPKALIFSASYRYTGVPDRFTGSGSGTDFTL
TISNVQSEDLAEYFCQQYDSYPLTFGGGTKLEIKRADAAPTVSIFPPSSEQLTSGGASVVCFLNNFYPKDINV
KWKIDGSERQNGVLNSWTDQDSKDSTYSMSSTLTLTKDEYERHNSYTCEATHKTSTSPIVKSFNRNEC
SEQ ID NO: 65
BioE2440 VL nucleic acid sequence
GACATCGTGATGACACAGTCTCAGAAGTTCATGAGCACAAGCGTGGGCGACAGAGTGAGCGTGACCTGCAAGG
CATCTCAGAACGTGGGCACCTACGTGGCCTGGTACGAGCAGAAGCTGGGGCAGAGCCCCAAGGCCCTGATCTT
CAGCGCAAGCTACAGATATACCGGCGTGCCCGACAGATTCACCGGCAGCGGCAGCGGCACCGACTTCACCCTG
ACCATCAGCAACGTGCAGAGCGAGGACCTGGCCGAGTACTTCTGTCAGCAGTACGACAGCTACCCCCTGACCT
TCGGCGGCGGCACCAAGCTCGAGATCAAGCGCGCAGATGCTGCTCCTACCGTGAGCATCTTCCCGCCGTCCAG
CGAACAACTCACTAGCGGAGGCGCGTCAGTGGTCTGCTTCCTTAACAATTTCTACCCTAAGGACATCAACGTC
AAGTGGAAGATTGACGGATCGGAACGCCAGAACGGAGTGCTGAACTCATGGACTGATCAGGATTCCAAAGACT
CGACTTACTCCATGTCCAGCACCCTGACCCTGACCAAAGACGAGTACGAAAGGCACAACTCGTACACGTGCGA
AGCCACCCACAAGACTTCCACCTCGCCCATCGTGAAGTCCTTCAATCGCAATGAGTGC
SEQ ID NO: 66
BioE2450 VH amino acid sequence
QVQLVESGGGVVQPGRSLRLSCAASGFTFSSYTMHWVRQAPGKGLEWVTFISYDGNNKYYADSVKGRFTISRD
NSKNTLYLQMNSLRAEDTAIYYCARTGWLGPFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTCPPCPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMHEALHNHYT
QKSLSLSPGK
SEQ ID NO: 67
BioE2450 VH nucleic acid sequence
CAAGTGCAGCTAGTTGAGTCTGGAGGCGGCGTGGTGCAGCCCGGCAGAAGCCTGAGACTGAGCTGCGCCGCAA
GCGGCTTCACCTTCAGCAGCTACACCATGCACTGGGTGAGACAAGCCCCCGGCAAGGGCCTGGAGTGGGTGAC
CTTCATCAGCTACGACGGCAACAACAAGTACTACGCCGACAGCGTGAAGGGCAGATTCACCATCAGCAGAGAC
AACAGCAAGAACACCCTGTACCTGCAGATGAACAGCCTGAGAGCCGAGGACACCGCCATCTACTACTGCGCTA
GAACCGGCTGGCTGGGCCCCTTCGACTACTGGGGCCAAGGCACCCTGGTAACGGTAAGCAGCGCTAGTACCAA
AGGGCCTAGCGTGTTTCCTTTAGCCCCTAGCAGCAAAAGCACAAGCGGAGGCACCGCCGCCCTGGGCTGTCTG
GTGAAGGATTACTTTCCTGAACCCGTGACTGTATCATGGAATAGCGGGGCCCTGACTAGCGGAGTGCATACCT
TCCCCGCCGTGCTGCAGAGCAGCGGCCTGTACAGCCTGAGCAGCGTGGTGACCGTGCCTAGCAGCAGCCTGGG
CACACAGACCTACATCTGCAACGTGAACCACAAGCCTAGCAACACCAAGGTGGACAAGAAGGTGGAGCCCAAG
AGCTGTGACAAAACGCACACCTGTCCGCCGTGCCCCGCCCCTGAGGCTGCCGGCGGCCCTAGCGTGTTCCTGT
TTCCCCCCAAGCCCAAGGACACCCTGATGATCAGCAGAACCCCTGAGGTGACCTGCGTGGTGGTGGACGTGAG
CCACGAGGACCCTGAGGTGAAGTTCAACTGGTACGTGGACGGCGTGGAGGTGCACAACGCCAAGACCAAGCCT
AGAGAGGAGCAGTACAACAGCACCTACAGAGTGGTGAGCGTGCTGACCGTGCTGCACCAAGACTGGCTGAACG
GCAAGGAGTACAAGTGCAAGGTGAGCAACAAGGCCCTGGGCGCCCCCATCGAGAAGACCATCAGCAAGGCCAA
GGGGCAGCCTAGAGAGCCCCAAGTGTACACCCTGCCCCCTAGCAGAGACGAGCTGACCAAGAACCAAGTGAGC
CTGACCTGCCTAGTGAAGGGTTTCTACCCTAGCGACATCGCCGTGGAGTGGGAGAGCAACGGGCAGCCTGAGA
ACAACTACAAGACCACCCCCCCCGTGCTGGACAGCGACGGCAGCTTCTTCCTGTACAGCAAGCTGACCGTGGA
CAAGAGCAGATGGCAGCAAGGCAACGTGTTCAGCTGCAGCGTGATGCACGAGGCCCTGCACAACCACTACACA
CAGAAGAGCCTGAGCCTGAGCCCCGGCAAG
SEQ ID NO: 68
BioE2450 VL amino acid sequence
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 69
BioE2450 VL nucleic acid sequence
GAGATCGTGCTGACACAGAGCCCCGGCACACTGTCTCTGAGCCCCGGCGAGAGAGCCACACTGAGCTGCAGAG
CCAGCCAGAGCGTGGGCAGCAGCTATCTGGCTTGGTACCAGCAGAAGCCCGGCCAAGCCCCTAGACTGCTGAT
TTACGGAGCCTTTAGCAGAGCCACCGGCATCCCCGACAGATTCAGCGGAAGCGGCAGCGGCACCGATTTCACA
CTGACAATCTCTAGACTGGAGCCAGAGGACTTCGCCGTGTACTACTGCCAGCAGTACGGCAGCAGCCCTTGGA
CCTTTGGCCAAGGCACCAAGGTGGAGATCAAGATCAAGAGAACTGTGGCCGCGCCGTCAGTGTTTATCTTCCC
TCCATCGGATGAACAGCTTAAGTCCGGCACGGCGTCTGTGGTCTGCCTGCTCAATAACTTTTACCCTAGGGAA
GCTAAAGTCCAATGGAAAGTGGATAACGCCCTGCAGTCAGGAAACAGCCAGGAATCGGTTACCGAACAGGACA
GCAAGGACAGCACTTACTCCTTGTCGTCGACTCTTACTCTGAGCAAGGCCGATTACGAGAAGCACAAGGTCTA
CGCCTGCGAGGTCACCCATCAGGGACTCTCGTCCCCGGTGACCAAATCCTTCAATAGAGGCGAATGC

TABLE 4
Representative VH and VL sequences of exemplaryCTLA4-binding proteins

| ID | Clone Name | | Amino acid sequence |
|---|---|---|---|
| cAb7125-1.1 | BNI3 Mouse | VH | SEQ ID NO: 70 |
| | IgG1 | | DVQLQESGPGLVKPSQSLSLTCSVTGYSITS |
| | | | GYVWNWIRQFPGNKLEWMGYISHDGNTN |
| | | | YNPSLKNRISITRDTSKNQFFLKLNSVTTEDS |
| | | | ATYYCTRNYGYGGTMDYWGQGTAVSVSS |
| | | VL | SEQ ID NO: 71 |
| | | | DIVMTQSQKFMSTSVGDRVSVTCKASQNV |
| | | | GTYVAWYEQKLGQSPKALIFSASYRYTGVP |
| | | | DRFTGSGSGTDFTLTISNVQSEDLAEYFCQQ |
| | | | YDSYPLTFGGGTKLEIK |
| cAb7125-10.0 | BNI3 | VH | SEQ ID NO: 72 |
| | Chimeric | | DVQLQESGPGLVKPSQSLSLTCSVTGYSITS |
| | human IgG1 | | GYVWNWIRQFPGNKLEWMGYISHDGNTN |
| | | | YNPSLKNRISITRDTSKNQFFLKLNSVTTEDS |
| | | | ATYYCTRNYGYGGTMDYWGQGTAVSVSS |
| | | VL | SEQ ID NO: 73 |
| | | | DIVMTQSQKFMSTSVGDRVSVTCKASQNV |
| | | | GTYVAWYEQKLGQSPKALIFSASYRYTGVP |
| | | | DRFTGSGSGTDFTLTISNVQSEDLAEYFCQQ |
| | | | YDSYPLTFGGGTKLEIK |
| cAb7126-10.0 | humBNI3-v1 | VH | SEQ ID NO: 74 |
| | | | DVQLQESGPGLVKPSQTLSLTCTVTGYSITS |
| | | | GYVWNWIRQPPGKGLEWMGYISHDGNTN |
| | | | YNPSLKSRVTISRDTSKNQFSLKLSSVTAAD |
| | | | TAVYYCTRNYGYGGTMDYWGQGTTVTVS |
| | | | S |
| | | VL | SEQ ID NO: 75 |
| | | | DIQMTQSPSSLSASVGDRVTITCRASQNVGT |
| | | | YVAWFQQKPGKAPKALIFSASYRYTGVPSR |
| | | | FSGSGSGTDFTLTISSLQPEDFATYFCQQYDS |
| | | | YPLTFGGGTKLEIK |
| cAb7127-10.0 | humBNI3-v2 | VH | SEQ ID NO: 76 |
| | | | DVQLQESGPGLVKPSQTLSLTCTVTGYSITS |
| | | | GYVWNWIRQPPGKGLEWMGYISHDGNTN |
| | | | YNPSLKSRVTISRDTSKNQFSLKLSSVTAAD |
| | | | TAVYYCTRNYGYGGTMDYWGQGTTVTVS |
| | | | S |
| | | VL | SEQ ID NO: 77 |
| | | | DIQMTQSPSSLSASVGDRVTITCRASQNVGT |
| | | | YVAWFQQKPGKAPKALIFSASYLYSGVPSR |
| | | | FSGSGSGTDFTLTISSLQPEDFATYYCQQYD |
| | | | SYPLTFGGGTKLEIK |
| cAb7128-10.0 | humBNI3-v3 | VH | SEQ ID NO: 78 |
| | | | DVQLQESGPGLVKPSQTLSLTCTVTGYSITS |
| | | | GYVWNWIRQPPGKGLEWMGYISHDGNTN |
| | | | YNPSLKSRVTISRDTSKNQFSLKLSSVTAAD |
| | | | TAVYYCTRNYGYGGTMDYWGQGTTVTVS |
| | | | S |
| | | VL | SEQ ID NO: 79 |
| | | | DIVMTQSPDSLAVSLGERATINCKASQNVG |
| | | | TYVAWYQQKPGQPPKALIFSASYRYTGVPD |
| | | | RFSGSGSGTDFTLTISSLQAEDVAVYFCQQY |
| | | | DSYPLTFGGGTKLEIK |
| cAb7129-10.0 | humBNI3-v4 | VH | SEQ ID NO: 80 |
| | | | DVQLQESGPGLVKPSQTLSLTCTVTGYSITS |
| | | | GYVWNWIRQPPGKGLEWMGYISHDGNTN |
| | | | YNPSLKSRVTISRDTSKNQFSLKLSSVTAAD |
| | | | TAVYYCTRNYGYGGTMDYWGQGTTVTVS |
| | | | S |
| | | VL | SEQ ID NO: 81 |
| | | | DIVMTQSPDSLAVSLGERATINCKASQNVG |
| | | | TYVAWYQQKPGQPPKALIFSASYRYSGVPD |
| | | | RFSGSGSGTDFTLTISSLQAEDVAVYYCQQY |
| | | | DSYPLTFGGGTKLEIK |
| cAb7130-10.0 | humBNI3-v5 | VH | SEQ ID NO: 82 |
| | | | QVQLQESGPGLVKPSQTLSLTCTVTGYSITS |
| | | | GYVWSWIRQPPGKGLEWIGYISHDGNTNY |
| | | | NPSLKSRVTISRDTSKNQFSLKLSSVTAADT |
| | | | AVYYCTRNYGYGGTMDYWGQGTTVTVSS |
| | | VL | SEQ ID NO: 83 |
| | | | DIQMTQSPSSLSASVGDRVTITCRASQNVGT |
| | | | YVAWFQQKPGKAPKALIFSASYRYTGVPSR |
| | | | FSGSGSGTDFTLTISSLQPEDFATYFCQQYDS |
| | | | YPLTFGGGTKLEIK |
| cAb7131-10.0 | humBNI3-v6 | VH | SEQ ID NO: 84 |
| | | | QVQLQESGPGLVKPSQTLSLTCTVTGYSITS |
| | | | GYVWSWIRQPPGKGLEWIGYISHDGNTNY |
| | | | NPSLKSRVTISRDTSKNQFSLKLSSVTAADT |
| | | | AVYYCTRNYGYGGTMDYWGQGTTVTVSS |
| | | VL | SEQ ID NO: 85 |
| | | | DIQMTQSPSSLSASVGDRVTITCRASQNVGT |
| | | | YVAWFQQKPGKAPKALIFSASYLYSGVPSR |
| | | | FSGSGSGTDFTLTISSLQPEDFATYYCQQYD |
| | | | SYPLTFGGGTKLEIK |
| cAb7132-10.0 | humBNI3-v7 | VH | SEQ ID NO: 86 |
| | | | QVQLQESGPGLVKPSQTLSLTCTVTGYSITS |
| | | | GYVWSWIRQPPGKGLEWIGYISHDGNTNY |
| | | | NPSLKSRVTISRDTSKNQFSLKLSSVTAADT |
| | | | AVYYCTRNYGYGGTMDYWGQGTTVTVSS |
| | | VL | SEQ ID NO: 87 |
| | | | DIVMTQSPDSLAVSLGERATINCKASQNVG |
| | | | TYVAWYQQKPGQPPKALIFSASYRYTGVPD |
| | | | RFSGSGSGTDFTLTISSLQAEDVAVYFCQQY |
| | | | DSYPLTFGGGTKLEIK |
| cAb7133-10.0 | humBNI3-v8 | VH | SEQ ID NO: 88 |
| | | | QVQLQESGPGLVKPSQTLSLTCTVTGYSITS |
| | | | GYVWSWIRQPPGKGLEWIGYISHDGNTNY |
| | | | NPSLKSRVTISRDTSKNQFSLKLSSVTAADT |
| | | | AVYYCTRNYGYGGTMDYWGQGTTVTVSS |
| | | VL | SEQ ID NO: 89 |
| | | | DIVMTQSPDSLAVSLGERATINCKASQNVG |
| | | | TYVAWYQQKPGQPPKALIFSASYRYSGVPD |
| | | | RFSGSGSGTDFTLTISSLQAEDVAVYYCQQY |
| | | | DSYPLTFGGGTKLEIK |
| cAb7134-10.0 | humBNI3-v9 | VH | SEQ ID NO: 90 |
| | | | DVQLKESGPALVKPTQTLTLTCTVTGYSITS |
| | | | GYVWNWIRQPPGKALEWMGYISHDGNTN |
| | | | YNPSLKTRLTISRDTSKNQFVLTMTNMDPV |
| | | | DTATYYCTRNYGYGGTMDYWGQGTTVTV |
| | | | SS |
| | | VL | SEQ ID NO: 91 |
| | | | DIQMTQSPSSLSASVGDRVTITCRASQNVGT |
| | | | YVAWFQQKPGKAPKALIFSASYRYTGVPSR |
| | | | FSGSGSGTDFTLTISSLQPEDFATYFCQQYDS |
| | | | YPLTFGGGTKLEIK |
| cAb7135-10.0 | humBNI3- | VH | SEQ ID NO: 92 |
| | v10 | | DVQLKESGPALVKPTQTLTLTCTVTGYSITS |
| | | | GYVWNWIRQPPGKALEWMGYISHDGNTN |
| | | | YNPSLKTRLTISRDTSKNQFVLTMTNMDPV |
| | | | DTATYYCTRNYGYGGTMDYWGQGTTVTV |
| | | | SS |
| | | VL | SEQ ID NO: 93 |
| | | | DIQMTQSPSSLSASVGDRVTITCRASQNVGT |
| | | | YVAWFQQKPGKAPKALIFSASYLYSGVPSR |
| | | | FSGSGSGTDFTLTISSLQPEDFATYYCQQYD |
| | | | SYPLTFGGGTKLEIK |
| cAb7136-10.0 | humBNI3- | VH | SEQ ID NO: 94 |
| | v11 | | DVQLKESGPALVKPTQTLTLTCTVTGYSITS |
| | | | GYVWNWIRQPPGKALEWMGYISHDGNTN |
| | | | YNPSLKTRLTISRDTSKNQFVLTMTNMDPV |
| | | | DTATYYCTRNYGYGGTMDYWGQGTTVTV |
| | | | SS |
| | | VL | SEQ ID NO: 95 |
| | | | DIVMTQSPDSLAVSLGERATINCKASQNVG |
| | | | TYVAWYQQKPGQPPKALIFSASYRYTGVPD |
| | | | RFSGSGSGTDFTLTISSLQAEDVAVYFCQQY |
| | | | DSYPLTFGGGTKLEIK |
| cAb7137-10.0 | humBNI3- | VH | SEQ ID NO: 96 |
| | v12 | | DVQLKESGPALVKPTQTLTLTCTVTGYSITS |
| | | | GYVWNWIRQPPGKALEWMGYISHDGNTN |
| | | | YNPSLKTRLTISRDTSKNQFVLTMTNMDPV |
| | | | DTATYYCTRNYGYGGTMDYWGQGTTVTV |
| | | | SS |
| | | VL | SEQ ID NO: 97 |
| | | | DIVMTQSPDSLAVSLGERATINCKASQNVG |
| | | | TYVAWYQQKPGQPPKALIFSASYRYSGVPD |
| | | | RFSGSGSGTDFTLTISSLQAEDVAVYYCQQY |
| | | | DSYPLTFGGGTKLEIK |
| cAb7138-10.0 | humBNI3- | VH | SEQ ID NO: 98 |
| | v13 | | QVTLKESGPALVKPTQTLTLTCTVTGYSITS |
| | | | GYVVSWIRQPPGKALEWIGYISHDGNTNYN |
| | | | PSLKTRLTISRDTSKNQFVLTMTNMDPVDT |
| | | | ATYYCTRNYGYGGTMDYWGQGTTVTVSS |
| | | VL | SEQ ID NO: 99 |
| | | | DIQMTQSPSSLSASVGDRVTITCRASQNVGT |
| | | | YVAWFQQKPGKAPKALIFSASYRYTGVPSR |
| | | | FSGSGSGTDFTLTISSLQPEDFATYFCQQYDS |
| | | | YPLTFGGGTKLEIK |
| cAb7139-10.0 | humBNI3- | VH | SEQ ID NO: 100 |
| | v14 | | QVTLKESGPALVKPTQTLTLTCTVTGYSITS |
| | | | GYVVSWIRQPPGKALEWIGYISHDGNTNYN |
| | | | PSLKTRLTISRDTSKNQFVLTMTNMDPVDT |
| | | | ATYYCTRNYGYGGTMDYWGQGTTVTVSS |
| | | VL | SEQ ID NO: 101 |
| | | | DIQMTQSPSSLSASVGDRVTITCRASQNVGT |
| | | | YVAWFQQKPGKAPKALIFSASYLYSGVPSR |
| | | | FSGSGSGTDFTLTISSLQPEDFATYYCQQYD |
| | | | SYPLTFGGGTKLEIK |
| cAb7140-10.0 | humBNI3- | VH | SEQ ID NO: 102 |
| | v15 | | QVTLKESGPALVKPTQTLTLTCTVTGYSITS |
| | | | GYVVSWIRQPPGKALEWIGYISHDGNTNYN |
| | | | PSLKTRLTISRDTSKNQFVLTMTNMDPVDT |
| | | | ATYYCTRNYGYGGTMDYWGQGTTVTVSS |
| | | VL | SEQ ID NO: 103 |
| | | | DIVMTQSPDSLAVSLGERATINCKASQNVG |
| | | | TYVAWYQQKPGQPPKALIFSASYRYTGVPD |
| | | | RFSGSGSGTDFTLTISSLQAEDVAVYFCQQY |
| | | | DSYPLTFGGGTKLEIK |
| cAb7141-10.0 | humBNI3- | VH | SEQ ID NO: 104 |
| | v16 | | QVTLKESGPALVKPTQTLTLTCTVTGYSITS |
| | | | GYVVSWIRQPPGKALEWIGYISHDGNTNYN |
| | | | PSLKTRLTISRDTSKNQFVLTMTNMDPVDT |
| | | | ATYYCTRNYGYGGTMDYWGQGTTVTVSS |
| | | VL | SEQ ID NO: 105 |
| | | | DIVMTQSPDSLAVSLGERATINCKASQNVG |
| | | | TYVAWYQQKPGQPPKALIFSASYRYSGVPD |
| | | | RFSGSGSGTDFTLTISSLQAEDVAVYYCQQY |
| | | | DSYPLTFGGGTKLEIK |

*Included in Tables 3 and 4 are nucleic acid molecules, e.g., RNA molecules (e.g., thymidines replaced with uridines), nucleic acid molecules encoding orthologs of the encoded proteins, as well as DNA, or cDNA; or nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Tables 3 and 4, or a portion thereof. Such nucleic acid molecules can have a function of the full-length nucleic acid.
*Included in Tables 3 and 4 are proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Tables 3 and 4, or a portion thereof. Such polypeptides can have a function of the full-length polypeptide.
*The polypeptide molecules or proteins of the present disclosure may further comprise an optional tag (e.g., His tag, etc.) and/or a leader sequence. An exemplary leader sequence may comprise the following sequence: MDFQVQIFSFLLISASVILSRG (SEQ ID NO: 55)
*Included in Tables 3 and 4 are proteins that comprise or lack an Fc domain. For proteins that comprise an Fc domain, these Tables include those comprising either the wild-type Fc domain or any variation thereof, e.g., those comprising a mutation (e.g., LALA mutation, LALAPG mutation, or any equivalent mutation known in the art), truncation. Such variation may have reduced or no binding to one or more Fc receptors.

### Nucleic Acids and Vectors

A further object of the invention relates to nucleic acid sequences encoding the antigen-binding proteins of the present invention.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, Gpenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

Accordingly, the nucleic acids of the present disclosure in some aspects are incorporated into a vector. In this regard, the present disclosure provides vectors comprising any of the presently disclosed nucleic acids. In various aspects, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector Is contacted with the cell under conditions sufficient to have the mRNA, protein, polypeptide, or peptide expressed within the cell. The vectors of the present disclosure are not naturally-occurring as a whole. However, parts of the vectors can be naturally-occurring. The presently disclosed vectors can comprise any type of nucleotides, including, but not limited to DNA and RNA, which can be single-stranded or double-stranded, synthesized or obtained in part from natural sources, and which can contain natural, non-natural or altered nucleotides. The vectors can comprise naturally-occurring or non-naturally-occurring internucleotide linkages, or both types of linkages. In some aspects, the altered nucleotides or non-naturally occurring internucleotide linkages do not hinder the transcription or replication of the vector.

The vector of the present disclosure can be any suitable vector, and can be used to transduce, transform or transfect any suitable host. Suitable vectors include those designed for propagation and expansion or for expression or both, such as plasmids and viruses. The vector can be a plasmid based expression vector. In various aspects, the vector is selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGTIO, λGTI 1, λZapII (Stratagene), λEMBL4, and λNMI 149, also can be used. Examples of plant expression vectors include pBIOI, pBI101.2, pBI101.3, pBI121 and pBIN19 (Clontech). Examples of animal expression vectors include pEUK-Cl, pMAM and pMAMneo (Clontech). In some aspects, the vector is a viral vector, e.g., a retroviral vector. In various aspects, the vector is an adenovirus vector, an adeno-associated virus (AAV) vector, a Herpes Simplex Virus (HSV) vector, a Vesicular stomatitis virus (VSV) vector, vaccinia virus vector, or lentivirus vector. See, e.g., Howarth et al., Cell Biol. Toxicol. 26 (1): 1-20 (2010). In various aspects, the vector is a baculovirus vector which infects arthropods, e.g., insects. In various aspects, the baculovirus vector is an Autographacalifornica multiple nuclear virus (AcMNPV) or a Bombyxmorinuclear polyhedrosis (BmNPV). See, e.g., Khan, Adv Pharm Bull 3 (2): 257-263 (2013); Miller, Bioessays 11 (4): 91-96 (1989); Atkinson et al., Pestic Sci 28:215-224 (1990).

The vectors of the present disclosure can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from CoIEl, 2 μ plasmid, λ, SV40, bovine papilloma virus, and the like.

In some aspects, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the presently disclosed expression vectors include, for instance, neomycin/G418 resistance genes, hygromycin resistance genes, histidinol resistance genes, tetracycline resistance genes, and ampicillin resistance genes.

The vector can comprise a native or normative promoter operably linked to the nucleotide sequence encoding the polypeptide (including functional portions and functional variants thereof), or to the nucleotide sequence which is complementary to or which hybridizes to the nucleotide sequence encoding the polypeptide. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

In another aspect, the present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody of the present invention. See, e.g., Ausubel, supra; Colligan, supra, each entirely incorporated herein by reference.

### Host Cells

Provided herein are host cells comprising a nucleic acid or vector of the present disclosure. A further object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids of the present invention may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL 1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described herein into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides of the invention.

As used herein, the term “host cell” refers to any type of cell that can contain the presently disclosed vector and is capable of producing an expression product encoded by the nucleic acid (e.g., mRNA, protein). The host cell in some aspects is an adherent cell or a suspended cell, i.e., a cell that grows in suspension. The host cell in various aspects is a cultured cell or a primary cell, i.e., isolated directly from an organism, e.g., a human. The host cell can be of any cell type, can originate from any type of tissue, and can be of any developmental stage.

In certain aspects, the antigen-binding protein is a glycosylated protein and the host cell is a glycosylation-competent cell. In various aspects, the glycosylation-competent cell is an eukaryotic cell, including, but not limited to, a yeast cell, filamentous fungi cell, protozoa cell, algae cell, insect cell, or mammalian cell. Such host cells are described in the art. See, e.g., Frenzel, et al., Front Immunol 4:217 (2013). In various aspects, the eukaryotic cells are mammalian cells. In various aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NSO, GS-NSO, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinoma human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3 (2): 257-263 (2013)).

For purposes of amplifying or replicating the vector, the host cell is in some aspects is a prokaryotic cell, e.g., a bacterial cell.

Also provided by the present disclosure is a population of cells comprising at least one host cell described herein. The population of cells in some aspects is a heterogeneous population comprising the host cell comprising vectors described, in addition to at least one other cell, which does not comprise any of the vectors. Alternatively, in some aspects, the population of cells is a substantially homogeneous population, in which the population comprises mainly host cells (e.g., consisting essentially of) comprising the vector. The population in some aspects is a clonal population of cells, in which all cells of the population are clones of a single host cell comprising a vector, such that all cells of the population comprise the vector. In various embodiments of the present disclosure, the population of cells is a clonal population comprising host cells comprising a vector as described herein.

In certain aspects the host cell is a human cell that is autologous or allogeneic to the subject. In some embodiments, a nucleic acid of the present invention is transduced via a viral vector or transformed in other suitable methods (e.g., electroporation, etc.). Such host cells are transferred (e.g., grafted, implanted, etc.) to the subject for a prolonged treatment of the disease or condition, e.g., cancer.

### Manufacturing Methods

Also provided herein are methods of producing an antigen-binding protein which binds to CTLA4. In various embodiments, the method comprises culturing a host cell comprising a nucleic acid comprising a nucleotide sequence encoding the antigen-binding protein as described herein in a cell culture medium and harvesting the antigen-binding protein from the cell culture medium. The host cell can be any of the host cells described herein. In various aspects, the host cell is selected from the group consisting of: CHO cells, NSO cells, COS cells, VERO cells, and BHK cells. In various aspects, the step of culturing a host cell comprises culturing the host cell in a growth medium to support the growth and expansion of the host cell. In various aspects, the growth medium increases cell density, culture viability and productivity in a timely manner. In various aspects, the growth medium comprises amino acids, vitamins, inorganic salts, glucose, and serum as a source of growth factors, hormones, and attachment factors. In various aspects, the growth medium is a fully chemically defined media consisting of amino acids, vitamins, trace elements, inorganic salts, lipids and insulin or insulin-like growth factors. In addition to nutrients, the growth medium also helps maintain pH and osmolality. Several growth media are commercially available and are described in the art. See, e.g., Arora, “Cell Culture Media: A Review” Mater Methods 3:175 (2013).

In various aspects, the method comprises culturing the host cell in a feed medium. In various aspects, the method comprises culturing in a feed medium in a fed-batch mode. Methods of recombinant protein production are known in the art. See, e.g., Li et al., “Cell culture processes for monoclonal antibody production” Mabs 2 (5): 466-477 (2010).

The method making an antigen-binding protein can comprise one or more steps for purifying the protein from a cell culture or the supernatant thereof and preferably recovering the purified protein. In various aspects, the method comprises one or more chromatography steps, e.g., affinity chromatography (e.g., protein A affinity chromatography, nickel resin for Histidine (His) tags), ion exchange chromatography, hydrophobic interaction chromatography. In various aspects, the method comprises purifying the protein using a Protein A affinity chromatography resin.

In various embodiments, the method further comprises steps for formulating the purified protein, etc., thereby obtaining a formulation comprising the purified protein. Such steps are described in Formulation and Process Development Strategies for Manufacturing, eds. Jameel and Hershenson, John Wiley & Sons, Inc. (Hoboken, NJ), 2010.

In various aspects, the antigen-binding protein linked to a polypeptide and the antigen-binding protein is part of a fusion protein. Thus, the present disclosure further provides methods of producing a fusion protein comprising an antigen-binding protein which binds to CTLA4. In various embodiments, the method comprises culturing a host cell comprising a nucleic acid comprising a nucleotide sequence encoding the fusion protein as described herein in a cell culture medium and harvesting the fusion protein from the cell culture medium.

Accordingly, the antigen-binding protein of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras or non-rodent-human chimeras) of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. The chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184, 187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988).J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In other embodiments, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. Humanized antibodies of the present invention can be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well-known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592, 106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

In addition, methods for producing antibody fragments are well-known. For example, Fab fragments of the present invention can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a prokaryote or eukaryote (as appropriate) to express the Fabs.

Similarly, F(ab′)2 fragments of the present invention can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present invention can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

The diabody molecules of the present invention can be produced using a variety of methods well known in the art, including de novo protein synthesis and recombinant expression of nucleic acids encoding the binding proteins. The desired nucleic acid sequences can be produced by recombinant methods or by solid-phase DNA synthesis. Exemplary methods of producing a diabody are known in the art (see e.g., U.S. Pat. Nos. 5,637,481A, 9,017,687B1, US20180194840A1).

### Modification of the Antigen-Binding Proteins

Amino acid sequence modification(s) of the antigen-binding proteins (e.g., antibody or fragments thereof, e.g., diabody, F(ab′)2), described herein are contemplated. For example, it may be desirable to improve the binding affinity and/or other biological properties of the (e.g., antibody or fragments thereof, e.g., diabody, F(ab′)2). It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Modifications and changes may be made in the structure of the antibodies of the present invention, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences that encode said polypeptides, without appreciable loss of their biological activity.

In some embodiments, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code (see genetic code chart above).

As described above, an important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It Is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antibody of the invention may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, I aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetyl galactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features an antigen-binding protein (e.g., antibody or fragments thereof, e.g., diabody, F(ab′)2) that specifically bind CTLA4 conjugated to a moiety that allows detection in vivo or in vitro. Conjugated antigen-binding protein can be used to monitor its presence in blood or tissues as part of a clinical testing procedure. Examples of detectable moieties include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance. For example, an antibody may be labeled with a nucleic acid sequence that may be amplified and detected, or an antisense oligonucleotide to reduce expression of a particular gene, such that expression can then be detected and measured.

Techniques for conjugating such therapeutic moiety to an antigen-binding protein (e.g., antibody or fragments thereof) are well-known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies ‘84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

### Conjugates

The present disclosure also provides antigen-binding proteins attached, linked or conjugated to a second moiety (e.g., a heterologous moiety, a conjugate moiety). Accordingly, the present disclosure provides a conjugate comprising an antigen-binding protein and a heterologous moiety. As used herein, the term “heterologous moiety” is synonymous with “conjugate moiety” and refers to any molecule (chemical or biochemical, naturally-occurring or non-coded) which is different from the antigen-binding proteins of the present disclosure. Various heterologous moieties include, but are not limited to, a polymer, a carbohydrate, a lipid, a nucleic acid, an oligonucleotide, a DNA or RNA, an amino acid, peptide, polypeptide, protein, therapeutic agent, (e.g., a cytotoxic agent, cytokine), or a diagnostic agent.

In some embodiments, the heterologous moiety is a polymer. The polymer can be branched or unbranched. The polymer can be of any molecular weight. The polymer in some embodiments has an average molecular weight of between about 2 kDa to about 100 kDa (the term “about” indicating that in preparations of a water soluble polymer, some molecules will weigh more, some less, than the stated molecular weight). The average molecular weight of the polymer is in some aspect between about 5 kDa and about 50 kDa, between about 12 kDa to about 40 kDa or between about 20 kDa to about 35 kDa.

In some embodiments, the polymer is modified to have a single reactive group, such as an active ester for acylation or an aldehyde for alkylation, so that the degree of polymerization can be controlled. The polymer in some embodiments is water soluble so that the protein to which it is attached does not precipitate in an aqueous environment, such as a physiological environment. In some embodiments, when, for example, the composition is used for therapeutic use, the polymer is pharmaceutically acceptable. Additionally, in some aspects, the polymer is a mixture of polymers, e.g., a co-polymer, a block co-polymer.

In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents.

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is a therapeutic agent. The therapeutic agent can be any of those known in the art. Examples of therapeutic agents that are contemplated herein include, but are not limited to, natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT+partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A2 inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopoptides, lincosamides, macrolies, oxazolidinones, penicillins, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamind reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opoid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The antigen-binding proteins of the present disclosure can be conjugated to one or more cytokines and growth factors that are effective in inhibiting tumor metastasis, and wherein the cytokine or growth factor has been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor a, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor a, transforming growth factor, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

The present disclosure also provides conjugates comprising an antigen-binding protein of the present disclosure linked to a polypeptide, such that the conjugate is a fusion protein. Therefore, the present disclosure provides fusion proteins comprising an antigen-binding protein of the present disclosure linked to a polypeptide. In various embodiments, the polypeptide is a diagnostic label, e.g., a fluorescent protein, such as green fluorescent protein, or other tag, e.g., Myc tag. In various aspects, the polypeptide is one of the cytokines, lymphokines, growth factors, or other hematopoietic factors listed above.

### Compositions, Pharmaceutical Compositions, and Formulations

Compositions comprising an antigen-binding protein, a nucleic acid, a vector, a host cell, or a conjugate as presently disclosed are provided herein. The compositions in some aspects comprise the antigen-binding proteins in isolated and/or purified form. In some aspects, the composition comprises a single type (e.g., structure) of an antigen-binding protein of the present disclosure or comprises a combination of two or more antigen-binding proteins of the present disclosure, wherein the combination comprises two or more antigen-binding proteins of different types (e.g., structures).

In some aspects, the composition comprises agents which enhance the chemico-physico features of the antigen-binding protein, e.g., via stabilizing the antigen-binding protein at certain temperatures, e.g., room temperature, increasing shelf life, reducing degradation, e.g., oxidation protease mediated degradation, increasing half-life of the antigen-binding protein, etc. In some aspects, the composition comprises any of the agents disclosed herein as a heterologous moiety or conjugate moiety, optionally in admixture with the antigen-binding proteins of the present disclosure or conjugated to the antigen-binding proteins.

In various aspects of the present disclosure, the composition additionally comprises a pharmaceutically acceptable carrier, diluents, or excipient. In some embodiments, the antigen-binding protein, a nucleic acid, a vector, a host cell, or a conjugate as presently disclosed (hereinafter referred to as “active agents”) is formulated into a pharmaceutical composition comprising the active agent, along with a pharmaceutically acceptable carrier, diluent, or excipient. In this regard, the present disclosure further provides pharmaceutical compositions comprising an active agent which is intended for administration to a subject, e.g., a mammal.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. In some embodiments, the compositions contain an active agent at a concentration of about 0.001 to about 30.0 mg/ml.

In various aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In various aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In various aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, Remington's Pharmaceutical Sciences, 18″ Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In various embodiments, the pH of the pharmaceutical composition is between 5.5 and 7.5.

The present disclosure provides methods of producing a pharmaceutical composition. In various aspects, the method comprises combining the antigen-binding protein, conjugate, fusion protein, nucleic acid, vector, host cell, or a combination thereof, with a pharmaceutically acceptable carrier, diluent, or excipient.

### Administration

With regard to the present disclosure, the active agent, or pharmaceutical composition comprising the same, can be administered to the subject via any suitable route of administration. For example, the active agent can be administered to a subject via parenteral, nasal, oral, pulmonary, topical, vaginal, or rectal administration. The following discussion on routes of administration is merely provided to illustrate various embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and I mixtures thereof.

The parenteral formulations in some embodiments contain from about 0.5% to about 25% by weight of the active agent of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceuties and Pharmacy Practice, J. B. Lippincott Company, Philadelphia, PA, Banker and Chalmers, eds., pages 238-250 (1982), and ASHP Handbook on Injectable Drugs, Toissel, 4^{th} ed., pages 622-630 (1986)).

### Dosages

The active agents of the disclosure are believed to be useful in methods of inhibiting tumor growth, as well as other methods, as further described herein, including methods of treating or preventing cancer. For purposes of the disclosure, the amount or dose of the active agent administered should be sufficient to effect, e.g., a therapeutic or prophylactic response, in the subject or animal over a reasonable time frame. For example, the dose of the active agent of the present disclosure should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of the active agent of the present disclosure to a mammal among a set of mammals, each set of which is given a different dose of the active agent, could be used to determine a starting dose to be administered to a mammal. The extent to which cancer is treated upon administration of a certain dose can be represented by, for example, the extent of tumor regression achieved with the active agent in a mouse xenograft model. Methods of assaying tumor regression are known in the art and described herein in the Examples.

The dose of the active agent of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

### Method of Preventing or Treating a Disease

The antigen-binding proteins of the present disclosure are useful for inhibiting tumor growth. Without being bound to a particular theory, the inhibiting action of the antigen-binding proteins provided herein allow such entities to be useful in methods of treating cancer.

In certain aspects, provided herein is a method of preventing or treating a subject afflicted with cancer, the method comprising administering to the subject an antigen-binding protein of the present disclosure, a combination of antigen-binding proteins, or a pharmaceutical composition comprising same.

In certain aspects, also provided herein is a method of reducing proliferation of a cancer cell in a subject in need thereof, the method comprising administering to the subject an antigen-binding protein of the present disclosure, a combination of antigen-binding proteins, or a pharmaceutical composition comprising same.

Further provided herein are methods of inhibiting tumor growth in a subject and methods of reducing tumor size in a subject. In various embodiments, the methods comprise administering to the subject the pharmaceutical composition of the present disclosure in an amount effective for inhibiting tumor growth or reducing tumor size in the subject.

In certain embodiments, the therapeutically effective amount of an antigen-binding protein, a combination of antigen-binding proteins, or pharmaceutical composition is administered to a subject in need thereof. In other embodiments, the cells that are autologous or allogeneic to the subject are obtained and transduced (e.g., via a viral vector, such as AAV) or otherwise transformed with a nucleic acid (or a vector comprising same) that encodes any one of the antigen-binding protein of the present disclosure. In preferred embodiments, such nucleic acid is stably integrated into the host cell genome. Upon confirming transformation of the nucleic acid, the cells are introduced to the subject (e.g., grafted or implanted) to supply a continued source of the antigen-binding proteins (i.e., expressed by the grafted cells and secreted into blood).

In various aspects, the growth of melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, or B-cell lymphoma is inhibited. In various aspects, the size of melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, or B-cell lymphoma is reduced.

As used herein, the term “inhibit” or “reduce” and words stemming therefrom may not be a 100% or complete inhibition or reduction. Rather, there are varying degrees of inhibition or reduction of which one of ordinary skill in the art recognizes as having a potential benefit or therapeutic effect. In this respect, the antigen-binding proteins of the present disclosure may inhibit tumor growth or reduce tumor size to any amount or level. In various embodiments, the inhibition provided by the methods of the present disclosure is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). In various embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In various aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject.

Numerous embodiments are further provided that can be applied to any aspect of the present invention and/or combined with any other embodiment described herein. For example, in some embodiments, an antigen-binding protein, a combination of antigen-binding proteins, or the pharmaceutical composition (a) reduces the number of proliferating cancer cells in the cancer; (b) reduces the volume or size of a tumor of the cancer; (c) increases the immune response against the cancer; and/or (d) activates the T cell.

In some embodiments, the method further comprises administering to the subject an additional cancer therapy. In some embodiments, the additional cancer therapy is selected from the group consisting of immunotherapy, checkpoint blockade, cancer vaccines, chimeric antigen receptors, chemotherapy, radiation, target therapy, and surgery, optionally wherein the additional cancer therapy is nivolumab.

In certain aspects, provided herein is a method of increasing an immune response in a subject, the method comprising administering to the subject an antigen-binding protein of the present disclosure, a combination of antigen-binding proteins, or a pharmaceutical composition comprising same.

In certain aspects, provided herein is a method of activating a T cell, the method comprising contacting the T cells with an antigen-binding protein of the present disclosure, a combination of antigen-binding proteins, or a pharmaceutical composition comprising same. Such method may be used in vivo, in vitro, or ex vivo.

In certain aspects, provided herein is a method of preventing or treating a disease or a condition characterized by aberrant expression or activity of a CTLA4 protein in a subject in need thereof, the method comprising administering to the subject an antigen-binding protein of the present disclosure or a pharmaceutical composition comprising same. In some embodiments, the disease or condition is a cancer, autoimmune disease, infection, or inflammatory disease.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In some embodiments, the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma, thyroid cancer, gall bladder cancer, head-and-neck (squamous) cancer, stomach (gastric) cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

### Cancer Therapy

The therapeutic agents of the present invention can be used alone or can be administered in combination therapy with, e.g., chemotherapeutic agents, hormones, antiangiogens, radiolabelled, compounds, or with surgery, cryotherapy, immunotherapy, cancer vaccine, immune cell engineering (e.g., CAR-T), and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer therapy one or more inhibitors of immune checkpoints (immune checkpoint inhibition therapy), such as PD1, PD-L1, and/or CD47 inhibitors. In some embodiments, the cancer therapy is nivolumab.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

The term “chimeric antigen receptor” or “CAR” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, I T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV and another protein binding fragments described herein.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NfkappaB signaling modulators, and immune checkpoint modulators, are used.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BlyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL 12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, various agents or a combination thereof can be used to treat a cancer. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of.beta.-nicotinamide adenine dinucleotide (NAD+) into nicotinamide and poly-ADP-ribose (PAR). Both poly(ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly (ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6^{th} edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

### Inflammatory Disorders

The antigen-binding proteins, a combination of antigen-binding proteins, and/or pharmaceutical compositions described herein can be used, for example, for preventing or treating (reducing, partially or completely, the adverse effects of) an inflammatory disease, such as chronic inflammatory bowel disease, systemic lupus erythematosus, psoriasis, muckle-wells syndrome, rheumatoid arthritis, multiple sclerosis, or Hashimoto's disease, an allergic disease, asthma; an infectious disease; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease. The antigen-binding proteins, a combination of antigen-binding proteins, and/or pharmaceutical compositions can be used for suppressing rejection in organ transplantation or other situations in which tissue rejection might occur; for improving immune functions; or for suppressing the proliferation or function of immune cells.

In certain embodiments, the inflammatory disorders include inflammation of any tissue and organs of the body, including musculoskeletal inflammation, vascular inflammation, neural inflammation, digestive system inflammation, ocular inflammation, inflammation of the reproductive system, and other inflammation.

In some embodiments, the musculoskeletal inflammation include conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

In some embodiments, the ocular immune disorders refers to an immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis

In some embodiments, the nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

In some embodiments, the digestive system immune disorders which may be treated with the methods and pharmaceutical compositions described herein include cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In some embodiments, the reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

In some embodiments, the inflammatory disorders include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, type 2 diabetes, giant cell arteritis, Goodpasture's syndrome, Graves' disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to treat T-cell mediated hypersensitivity diseases having an inflammatory component. Such conditions include contact hypersensitivity, contact dermatitis (including that due to poison ivy), uticaria, skin allergies, respiratory allergies (hay fever, allergic rhinitis, house dust mite allergy) and gluten-sensitive enteropathy (Celiac disease).

Other immune disorders which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Clinical Efficacy/Response to a Therapy

Clinical efficacy can be measured by any method known in the art. For example, the response to a therapy relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before Initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007)25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR-CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using methods such as those described in the Examples section.

### Kits

In some embodiments, the antigen-binding proteins of the present disclosure are provided in a kit. In various aspects, the kit comprises the antigen-binding protein(s) as a unit dose. For purposes herein “unit dose” refers to a discrete amount dispersed in a suitable carrier. In various aspects, the unit dose is the amount sufficient to provide a subject with a desired effect, e.g., inhibition of tumor growth, reduction of tumor size, treatment of cancer. Accordingly, provided herein are kits comprising an antigen-binding protein of the present disclosure optionally provided in unit doses. In various aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the antigen-binding protein of the present disclosure, a pharmaceutically acceptable salt thereof, a conjugate comprising the antigen-binding protein, or a multimer or dimer comprising the antigen-binding protein, is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein. In particular aspects, the kit comprises an antigen-binding protein of the present disclosure, along with an agent, e.g., a therapeutic agent, used in chemotherapy or radiation therapy.

### Exemplary Embodiments

1. An antigen-binding protein, comprising:
a) a heavy chain variable domain (VH) amino acid sequence set forth in Table 4, or a variant sequence thereof which differs by only one or two amino acids or which has at least or about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% sequence identity; and/or
b) a light chain variable domain (VL) amino acid sequence set forth in Table 4, or a variant sequence thereof which differs by only one or two amino acids or which has at least or about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% sequence identity.

2. The antigen-binding protein of 1, wherein the antigen-binding protein comprises the VH and VL amino acid sequences set forth in:
a) SEQ ID NO: 72 and SEQ ID NO: 73;
b) SEQ ID NO: 74 and SEQ ID NO: 75;
c) SEQ ID NO: 76 and SEQ ID NO: 77;
d) SEQ ID NO: 78 and SEQ ID NO: 79;
e) SEQ ID NO: 80 and SEQ ID NO: 81;
f) SEQ ID NO: 82 and SEQ ID NO: 83;
g) SEQ ID NO: 84 and SEQ ID NO: 85;
h) SEQ ID NO: 86 and SEQ ID NO: 87;
i) SEQ ID NO: 88 and SEQ ID NO: 89;
j) SEQ ID NO: 90 and SEQ ID NO: 91;
k) SEQ ID NO: 92 and SEQ ID NO: 93;
l) SEQ ID NO: 94 and SEQ ID NO: 95;
m) SEQ ID NO: 96 and SEQ ID NO: 97;
n) SEQ ID NO: 98 and SEQ ID NO: 99;
o) SEQ ID NO: 100 and SEQ ID NO: 101;
p) SEQ ID NO: 102 and SEQ ID NO: 103;
q) SEQ ID NO: 104 and SEQ ID NO: 105; or
   a variant sequence thereof which differs by only one or two amino acids or which has at least or about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% sequence identity.

3 The antigen-binding protein of 1 or 2, wherein the antigen-binding protein
a) binds specifically to CTLA4; and/or
b) blocks the interaction between CTLA4 and its ligands (e.g., CD80 (B7-1) and CD86 (B7-2)).

4. The antigen-binding protein of any one of 1-3, wherein the antigen-binding protein does not comprise an Fc domain.

5. The antigen-binding protein of any one of 1-4, wherein the antigen-binding protein comprises an Fc domain, optionally wherein the Fc domain is a human IgG1 Fc.

6. The antigen-binding protein of any one of 1-3 and 5, wherein the antigen-binding protein comprises an immunoglobulin heavy chain constant domain selected from the IgG, IgG1, IgG2, IgG2A, IgG2B, IgG3, IgG4, IgA, IgM, IgD, and IgE constant domains.

7. The antigen-binding protein of any one of 1-6, wherein the antigen-binding protein does not bind to one or more Fc receptors.

8 The antigen-binding protein of any one of 5-7, wherein the Fc domain comprises a LALAPG mutation or a LALA mutation.

9. The antigen-binding protein of any one of 1-8, wherein the antigen-binding protein is selected from an antibody, Fv, F(ab′)2, Fab′, dsFv, scFv, sc (Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

10. The antigen-binding protein of any one of 1-9, wherein the antigen-binding protein is an antibody, optionally a monoclonal antibody.

11. The antigen-binding protein of any one of 1-10, wherein the antigen-binding protein is chimeric, humanized, composite, murine, or human, optionally wherein the antigen-binding protein is humanized.

12. The antigen-binding protein of any one of 1-11, wherein the antigen-binding protein is:
a) an IgG1 monoclonal antibody; or
b) an IgG1 monoclonal antibody comprising a LALAPG mutation in the Fc region.

13. The antigen-binding protein of any one of 1-12, wherein the antigen-binding protein is conjugated or detectably labeled.

14. The antigen-binding protein of any one of 1-13, wherein the antigen-binding protein further comprises a polymer or a heterologous polypeptide (e.g., an enzyme, a half-life extender (e.g., human serum albumin), detectable polypeptide (e.g., GFP)).

15. The antigen-binding protein of any one of 1-14, wherein the heterologous polypeptide comprises a peptide tag (e.g., His6 tag) and/or a leader sequence.

16. The antigen-binding protein of 14 or 15, wherein the polymer or the heterologous polypeptide extends a half-life of the antigen-binding protein.

17. The antigen-binding protein of any one of 14-16, wherein the heterologous polypeptide comprises an albumin-binding protein, albumin, an Fc domain, a fragment of an Fc domain, an FcRnBP.

18. The antigen-binding protein of 14 or 16, wherein the polymer comprises polyethylene glycol (PEG) or a variant thereof (e.g., glycol-PEG).

19. An isolated nucleic acid that encodes the antigen-binding protein of any one of 1-18.

20. A vector comprising the isolated nucleic acid of 19.

21. A host cell which comprises the isolated nucleic acid of 19, comprises the vector of 20, and/or expresses the antigen-binding protein of any one of 1-18.

22. A pharmaceutical composition comprising the antigen-binding protein of any one of 1-18, an isolated nucleic acid of 19, a vector of 20, and/or a host cell of 21.

23. The pharmaceutical composition of 22, further comprising an antigen-binding protein that binds PD-1 and/or PD-L1.

24. The pharmaceutical composition of 23, wherein the antigen-binding protein that binds PD-1 or PD-L1 is selected from nivolumab, pembrolizumab, atezolizumab, avelumab, durvalumab, cemiplimab, and dostarlimab.

25. An antigen-binding protein comprising an Fc domain, wherein the antigen-binding protein does not bind to one or more Fc receptors, and comprises the VH and VL domain amino acid sequences set forth in:
a) SEQ ID Nos: 12 and 14;
b) SEQ ID Nos: 15 and 17; or
   a variant sequence thereof which differs by only one or two amino acids or which has at least or about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% sequence identity.

26. The antigen-binding protein of 25, wherein the antigen-binding protein
a) binds specifically to CTLA4; and/or
b) blocks the interaction between CTLA4 and its ligands (e.g., CD80 (B7-1) and CD86 (B7-2)).

27. The antigen-binding protein of 25 or 26, wherein the antigen-binding protein comprises an immunoglobulin heavy chain constant domain selected from the IgG, IgG1, IgG2, IgG2A, IgG2B, IgG3, IgG4, IgA, IgM, IgD, and IgE constant domains. 28. The antigen-binding protein of any one of 25-27, wherein the antigen-binding protein is an antibody, optionally a monoclonal antibody.

29. The antigen-binding protein of any one of 25-28, wherein the antigen-binding protein is chimeric, humanized, composite, murine, or human, optionally wherein the antigen-binding protein is humanized.

30. The antigen-binding protein of any one of 25-29, wherein the Fc domain comprises a LALAPG mutation or a LALA mutation.

31. The antigen-binding protein of any one of 25-30, wherein the antigen-binding protein is a human IgG1 monoclonal antibody comprising a LALAPG mutation in the Fc region.

32. The antigen-binding protein of any one of 25-31, wherein the antigen-binding protein is conjugated or detectably labeled.

33. The antigen-binding protein of any one of 25-32, wherein the antigen-binding protein further comprises a polymer or a heterologous polypeptide.

34. The antigen-binding protein of 33, wherein the heterologous polypeptide comprises a peptide tag (e.g., His6 tag) and/or a leader sequence.

35. The antigen-binding protein of 33 or 34, wherein the polymer or the heterologous polypeptide extends a half-life of the antigen-binding protein.

36. The antigen-binding protein of any one of 33-35, wherein the heterologous polypeptide comprises an albumin-binding protein, albumin, a fragment of an Fc domain, or an FcRnBP.

37. The antigen-binding protein of 33 or 35, wherein the polymer comprises polyethylene glycol (PEG) or a variant thereof (e.g., glycol-PEG).

38. A pharmaceutical composition comprising the antigen-binding protein of any one of 25-37.

39. The pharmaceutical composition of 38, further comprising an antigen-binding protein that binds PD-1 and/or PD-L1.

40. A pharmaceutical composition comprising an antigen-binding protein that binds PD-1 or PD-L1; and an antigen-binding protein comprising the VH and VL amino acid sequences set forth in:
a) SEQ ID NOs: 12 and 14;
b) SEQ ID NOs: 12, 14, 15, and 17;
c) SEQ ID NO: 24, or
a variant sequence thereof which differs by only one or two amino acids or which has at least or about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% sequence identity.

41. The pharmaceutical composition of 39 or 40, wherein the antigen-binding protein that binds PD-1 or PD-L1 is selected from nivolumab, pembrolizumab, atezolizumab, avelumab, durvalumab, cemiplimab, and dostarlimab.

42. A composition comprising at least two antigen-binding proteins that specifically bind cytotoxic T-lymphocyte-associated antigen-4 (CTLA4).

43. The composition of 42, wherein the at least two antigen-binding proteins bind different epitopes of CTLA4.

44. The composition of 43, wherein the epitope of CTLA4 is selected from the residues ¹³⁴MYPPPY¹³⁹, the residues ⁶⁵SICT⁶⁸, and the residues ⁵⁸ELT⁶⁰ of CTLA4.

45. The composition of any one of 42-44, wherein the at least one antigen-binding protein blocks the interaction between CTLA4 and its ligands (e.g., CD80 (B7-1) and CD86 (B7-2)).

46. The composition of any one of 42-45, wherein at least one antigen-binding protein does not comprise an Fc domain.

47. The composition of any one of 42-46, wherein at least one antigen-binding protein comprises an Fc domain, optionally wherein the Fc domain is a human IgG1 Fc.

48. The composition of any one of 42-45 and 47, wherein at least one antigen-binding protein comprises an immunoglobulin heavy chain constant domain selected from the IgG, IgG1, IgG2, IgG2A, IgG2B, IgG3, IgG4, IgA, IgM, IgD, and IgE constant domains.

49. The composition of any one of 42-48, wherein at least one antigen-binding protein does not bind to one or more Fc receptors.

50. The composition of any one of 42-49, wherein at least one antigen-binding protein comprises the Fc domain comprising a LALAPG mutation or a LALA mutation.

51. The composition of any one of 42-50, wherein at least one antigen-binding protein is selected from an antibody, Fv, F(ab′)2, Fab′, dsFv, scFv, sc (Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

52. The composition of any one of 42-51, wherein at least one antigen-binding protein is an antibody, optionally a monoclonal antibody.

53. The composition of any one of 42-52, wherein at least one antigen-binding protein is chimeric, humanized, composite, murine, or human, optionally wherein the antigen-binding protein is humanized.

54. The composition of any one of 42-53, wherein at least one antigen-binding protein is:
a) an IgG1 monoclonal antibody; or
b) an IgG1 monoclonal antibody comprising a LALAPG mutation in the Fc region.

55. The composition of any one of 42-54, wherein at least one antigen-binding protein is conjugated or detectably labeled.

56. The composition of any one of 42-55, wherein at least one antigen-binding protein further comprises a polymer or a heterologous polypeptide.

57. The composition of 56, wherein the heterologous polypeptide comprises a peptide tag (e.g., His6 tag) and/or a leader sequence.

58. The composition of 56 or 57, wherein the polymer or the heterologous polypeptide extends a half-life of the antigen-binding protein.

59. The composition of any one of 56-58, wherein the heterologous polypeptide comprises an albumin-binding protein, albumin, or an Fc domain.

60. The composition of 56 or 58, wherein the polymer comprises polyethylene glycol (PEG) or a variant thereof (e.g., glycol-PEG).

61. The composition of any one of 42-60, wherein at least one antigen-binding protein is selected from the antigen-binding proteins of any one of 1-18, 25-37, and 40.

62. The composition of any one of 42-61, wherein at least two antigen-binding proteins are selected from the antigen-binding proteins of any one of 1-18, 25-37, and 40.

63. The composition of any one of 42-62, wherein at least one antigen-binding protein is selected from the antigen-binding proteins listed in Table 6 and/or Table 7.

64. The composition of any one of 42-63, wherein at least two antigen-binding proteins are selected from the antigen-binding proteins listed in Table 6 and/or Table 7.

65. The composition of any one of 42-64, wherein the composition comprises:
a) an antigen-binding protein comprising the VH and VL amino acid sequences selected from SEQ ID NOs: 72-105;
b) an antigen-binding protein comprising the VH and VL amino acid sequences set forth in SEQ ID NOs: 12 and 14;
c) an antigen-binding protein comprising the VH and VL amino acid sequences set forth in SEQ ID NOs: 15 and 17;
d) an antigen-binding protein comprising the VH and VL amino acid sequences set forth in SEQ ID NOs: 12, 14, 15, and 17;
e) an antigen-binding protein comprising the sequence set forth in SEQ ID NO: 24; or
f) two antigen-binding proteins comprising any combination of a)-e).

66. The composition of any one of 42-65, wherein composition comprises:
a) an antigen-binding protein comprising the VH and VL amino acid sequences selected from SEQ ID NOs: 72-105; and
b) an antigen-binding protein comprising the VH and VL amino acid sequences set forth in SEQ ID NOs: 12 and 14.

67. The composition of any one of 42-65, wherein composition comprises:
a) an antigen-binding protein comprising the VH and VL amino acid sequences selected from SEQ ID NOs: 72-105; and
b) an antigen-binding protein comprising the VH and VL amino acid sequences set forth in SEQ ID NOs: 15 and 17.

68. The composition of any one of 42-65, wherein composition comprises:
a) an antigen-binding protein comprising the VH and VL amino acid sequences selected from SEQ ID NOs: 72-105; and
b) an antigen-binding protein comprising the VH and VL amino acid sequences set forth in SEQ ID NOs: 12, 14, 15, and 17; or an antigen-binding protein comprising the sequence set forth in SEQ ID NO: 24.

69. The composition of any one of 42-68, wherein at least one antigen-binding protein is selected from an antibody, an antibody comprising a LALAPG mutation or a LALA mutation in its Fc domain, F(ab′)2, and Fab′.

70. The composition of any one of 42-69, wherein the composition comprises two antigen-binding proteins that specifically bind CTLA4.

71. The composition of 70, wherein the two antigen-binding proteins are present at an equimolar concentration, or not present at an equimolar concentration.

72. The composition of 70 or 71, wherein the molar ratio of one antigen-binding protein to another antigen-binding protein is at least or about 1:1000, 1:100, 1:10, or 1:1.

73. The composition of any one of 42-72, further comprising an antigen-binding protein that binds PD-1 and/or PD-L1.

74. The composition of 73, wherein the antigen-binding protein that binds PD-1 or PD-L1 is selected from nivolumab, pembrolizumab, atezolizumab, avelumab, durvalumab, cemiplimab, and dostarlimab.

75. A pharmaceutical composition comprising the composition of any one of 42-74.

76. A kit comprising an antigen-binding protein of any one of 1-18 and 25-37; a composition of any one of 42-74; or a pharmaceutical composition of any one of 22-24, 38-41, and 75.

77. A method of producing the antigen-binding protein of any one of 1-18 and 25-37, wherein the method comprises the steps of: (i) culturing a host cell comprising a nucleic acid comprising a sequence encoding the antigen-binding protein of any one of 1-18 and 25-37 under conditions suitable to allow expression of said antigen-binding protein; and (ii) recovering the expressed antigen-binding protein.

78. A method of preventing or treating a subject afflicted with a cancer, the method comprising administering to the subject at least one selected from: an antigen-binding protein of any one of 1-18 and 25-37; a composition of any one of 42-74; and a pharmaceutical composition of any one of 22-24, 38-41, and 75.

79. A method of inhibiting proliferation of a cancer cell in a subject, the method comprising administering to the subject at least one selected from: an antigen-binding protein of any one of 1-18 and 25-37; a composition of any one of 42-74; and a pharmaceutical composition of any one of 22-24, 38-41, and 75.

80. The method of 78 or 79, wherein if two or more of antigen-binding proteins, compositions, or pharmaceutical compositions are administered to the subject, then the two or more of antigen-binding proteins, compositions, or pharmaceutical compositions are administered conjointly to the subject.

81. The method of 80, wherein an antigen-binding protein that binds PD-1 or PD-L1 is administered to the subject conjointly with:
a) an antigen-binding protein of any one of 1-18 and 25-37; and/or
b) an antigen-binding protein comprising the sequences set forth in:
   i) SEQ ID NOs: 12 and 14;
   ii) SEQ ID NOs: 12, 14, 15, and 17;
   iii) SEQ ID NO: 24, or
   iv) a variant sequence of any one of i)-iii) which differs by only one or two amino acids or which has at least or about 80%, 81%, 82%, 83%, 84%, 85%, 86%, 87%, 88%, 89%, 90%, 91%, 92%, 93%, 94%, 95%, 96%, 97%, 98%, 99%, 99.5%, or 100% sequence identity.

82. The method of 80, wherein the subject is administered conjointly with two or more of antigen-binding proteins selected from:
a) an antigen-binding protein comprising the VH and VL amino acid sequences selected from SEQ ID NOs: 72-105;
b) an antigen-binding protein comprising the VH and VL amino acid sequences set forth in SEQ ID NOs: 12 and 14;
c) an antigen-binding protein comprising the VH and VL amino acid sequences set forth in SEQ ID NOs: 15 and 17;
d) an antigen-binding protein comprising the VH and VL amino acid sequences set forth in SEQ ID NOs: 12, 14, 15, and 17; and
e) an antigen-binding protein comprising the sequence set forth in SEQ ID NO: 24.

83. The method of 82, wherein the subject is further administered conjointly with an 83. antigen-binding protein that binds PD-1 or PD-L1.

84. The method of 81 or 83, wherein the antigen-binding protein that binds PD-1 or PD-L1 is selected from nivolumab, pembrolizumab, atezolizumab, avelumab, durvalumab, cemiplimab, and dostarlimab.

85. The method of any one of 78-84, wherein the antigen-binding protein, the composition, or the pharmaceutical composition (a) decreases the number of proliferating cancer cells; (b) reduces the volume or size of a tumor of the cancer; (c) increases the immune response against the cancer; and/or (d) activates a T cell.

86. The method of any one of 78-84, further comprising conjointly administering to the subject an additional cancer therapy.

87. The method of 86, wherein the additional cancer therapy is selected from the group consisting of immunotherapy, checkpoint blockade, cancer vaccines, chimeric antigen receptors, chemotherapy, radiation, target therapy, and surgery, optionally wherein the additional cancer therapy is checkpoint blockade.

88. The method of any one of 78-87, wherein the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma, thyroid cancer, gall bladder cancer, head-and-neck (squamous) cancer, stomach (gastric) cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

89. The method of any one of 78-88, wherein the cancer is selected from melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, and B-cell lymphoma.

90. A method of increasing an immune response in a subject, the method comprising administering to the subject at least one selected from: an antigen-binding protein of any one of 1-18 and 25-37; a composition of any one of 42-74; and a pharmaceutical composition of any one of 22-24, 38-41, and 75.

91. A method of activating a T cell, the method comprising contacting the T cell with at least one selected from: an antigen-binding protein of any one of 1-18 and 25-37; a composition of any one of 42-74; or a pharmaceutical composition of any one of 22-24, 38-41, and 75.

92. A method of preventing or treating a disease or a condition characterized by aberrant expression or activity of a CTLA4 protein in a subject, the method comprising administering to the subject at least one selected from an antigen-binding protein of any one of 1-18 and 25-37; a composition of any one of 42-74; or a pharmaceutical composition of any one of 22-24, 38-41, and 75.

93. The method of 92, wherein the disease or condition is a cancer, autoimmune disease, infection, or inflammatory disease.

94. The method of any one of 78-93, wherein the subject is a mammal, optionally a mouse, a dog, or a cat, or a human.

95. An antibody comprising at least one CDR derived from any one of VH or VL domains disclosed herein.

96. An antibody comprising (a) 3 CDRs derived from any one of VH domains disclosed herein; and/or (b) 3 CDRs derived from any one of VL domains disclosed herein. A person of ordinary skill in the art can derive CDR sequences from a known VH or VL domain using any one or more of the methods known in the art. The most commonly used numbering schemes include IMGT, Kabat, Chothia, Martin (Enhanced Chothia or AbM) and Honneger's numbering scheme (AHo). The Kabat definition is based on sequence variability and is the most commonly used. The Chothia definition is based on the location of the structural loop regions. The AbM definition is a compromise between the two used by Oxford Molecular's AbM antibody modelling software. The contact definition is based on an analysis of the available complex crystal structures. Additional information regarding determining CDR sequences can be found, for example, in World Wide Web at bioinf.org.uk/abs/info.html.

The following examples are given merely to illustrate the present disclosure and not in any way to limit its scope.

### EXAMPLES

### Example 1: Functional Assays

Three in vitro functional blockade assays were used herein to (i) identify and characterize anti-CTLA4 antibodies or combinations thereof, and (ii) their role in inflammation and cancer biology.

### CTLA4 Functional Blockade Assay

As used herein, the CTLA4 blockade assay determines the activation of T cells by one or more anti-CTLA4 antibodies.

The CTLA4 blockade assay described herein used the commercially available kit, CTLA4 blockade bioassay (cat. #JA3001 and JA3005; Promega Corporation, Madison, WI). CTLA4, also known as CD152, is an immune inhibitory receptor constitutively expressed on regulatory T cells (Tregs) and upregulated in activated T cells. CTLA4 plays a critical role in regulating immune responses to tumor antigens and autoantigens. CTLA4 is the counterpart of the co-stimulatory B7-CD28 pathway. When CTLA4 expression is upregulated on the surface of T cells, the T cells bind B7 with a higher avidity, and thus out-compete the positive co-stimulatory signal from CD28. In addition, engagement of CTLA4 by either of its ligands, CD80 (B7-1) or CD86 (B7-2) on an adjacent antigen presenting cell (APC) inhibits CD28 co-stimulation of T cell activation, cell proliferation and cytokine production.

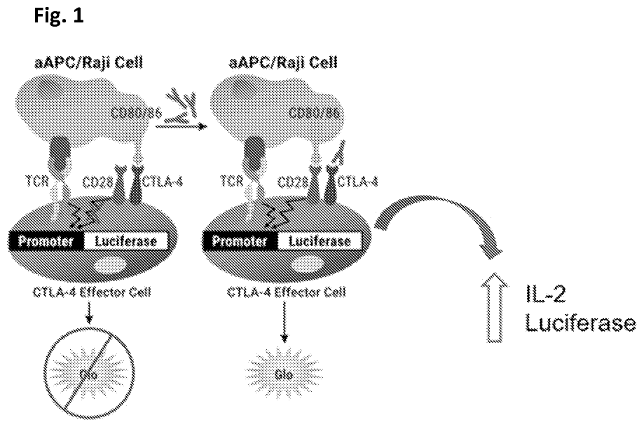

LUMINESCENCE READOUT: The CTLA4 blockade assay involved two cell lines: Jurkat T cells (immortalized lymphocytic leukemia T cells, also referred to as the CTLA4 effector cells) and Raji cells (also referred to as the antigen-presenting cells (APC)). Jurkat T cells express human CTLA4 and a luciferase reporter driven by a native promoter which responds to TCR/CD28 activation. Raji cells express an engineered cell surface protein designed to activate cognate TCRs in an antigen-independent manner and endogenously expressing CTLA4 ligands CD80 (B7-1) and CD86 (B7-2), collectively called the B7 ligands. When the two cell types are co-cultured, CTLA4 competes with CD28 for their shared ligands, CD80 and CD86, and thus inhibits CD28 pathway activation and promoter-mediated luminescence. Addition of an anti-CTLA4 antibody blocks the interaction of CTLA4 with its ligands CD80 and CD86 and results in promoter-mediated luminescence (FIG. 1).

For comparative analysis purposes, the ratio of the Test Agent Relative Luciferase Units (RLU) of Activity to the No Test Agent Relative Luciferase Units (RLU) was used to standardize the Jurkat response to CTLA 4 blockade. Typically, we found Ipilimumab to have ratios of 15+/−2 relative to a no test agent response (1+/−0.15).

IL-2 READOUT: Jurkat cells can also be activated by anti-CD3, anti-CD28, or anti-CD3 with cell surface B7 ligands to express IL-2. Thus, in addition to the luminescence, the expression level of IL-2 protein is a metric of activation. Accordingly, when Jurkat cells are co-cultured with Raji cells that endogenously express B7 ligands and now engineered to express anti-CD3, the Jurkat cells are activated and express both IL-2 and luciferase. The expression levels of IL-2 and luciferase are linearly correlated with each other and with activation. However, the presence of CTLA4 extracellular domain, which binds the B7 ligands and blocks the Jurkat cell activation, abrogates both IL-2 and luciferase expression. Agents that block CTLA4 function, e.g., blocking the CTLA4's binding of B7 ligands, enable the Jurkat cell expression of IL-2 and luciferase. There is a linear correlation between IL-2 expression, luciferase expression/activity, and blocking efficacy of the anti-CTLA4 agent. The correlation between the CTLA4 blockade as measured by this assay and the primary T cell activation has been well documented, further validating the applicability of this assay (Waight et al. (2018) Cancer Cell 33:1033-1047).

Anti-CTLA4 antibodies in certain in vitro T cell activation settings with antigen presenting cells show a biological effect dependent on Fc receptor interactions. Specifically, the interaction between the Fc region of a CTLA4 antibody with the Fc receptors is important for its CTLA4-blocking activity (Bulliard et al. (2013) J of Exp Medicine 9:1685-1693; Waight et al. (2018) Cancer Cell 33:1033-1047; Ingram et al. (2018) Proc Natl Acad Sci USA 115:3912-3917; Vargas et al. (2018) Cancer Cell 33:1-15). This is reflected in the CTLA4 bloackade assay. Raji cells express FcγRII (CD32), and blockade of anti-CTLA4 antibody Fc interactions with FcγRII through either an anti-CD32 antibody or through deletion of Fc domains leads to an attenuation of an anti-CTLA4 antibody's activity and a decrease in the absolute level of response. This attenuation of an anti-CTLA4 antibody's activity has been reported to extend to antigen stimulation of primary T cell activation, where the FcγRIIIA (CD16) receptor was essential and not FcγRI, FcγRIIA/B, or FcγRIIB (Waight et al. (2018) Cancer Cell 33:1033-1047). However, Raji cells used in the Promega assay do not express FcγRIIIA (CD16) and no CD16-dependent effect on the anti-CTLA4 antibody activity is observed, i.e., the presence of anti-CD16 antibody does not attenuate the anti-CTLA4 antibody activity.

The Promega CTLA 4 Blockade Bioassay (JA3001, JA3005) was processed as described in the Promega Technical Manual. The assay involved 120 samples on 2 plates of the 96-well plate. The assay was performed with samples in triplicates, in a titration curve of the protein concentration. Routinely, unless otherwise noted, concentrations of the samples and standard control anti-CTLA 4 antibodies were also assayed in triplicates in a titration curve of the protein concentration, and were tested concurrently in the sample test plate. The Promega CTLA4 Blockade Bioassay is designed and fitted for a Relative Luciferase Activity (RLU) readout. The activities of samples were reported herein as the ratio of Test Agent Relative Luciferase Units/No Test Agent Relative Luciferase Units (background luciferase units representative of basal levels of T cell activation). The assays were incubated from 12 to 16 hours. While variation in Relative Luciferase Units occurred among experiments, but the ratios of the CTLA4-blocking activity remained constant. In some experiments, rather than measuring luciferase activity, the assay well media was assayed for human IL-2 levels. Conventional Human IL-2 ELISA assays were used. Generally, the entire contents of the assay well were analyzed directly for IL-2.

The control anti-human CTLA4 antibodies used were: Cat. No. JA1020 from Promega, ipilimumab from SelleckChem, and L3D10 from BioLegends.

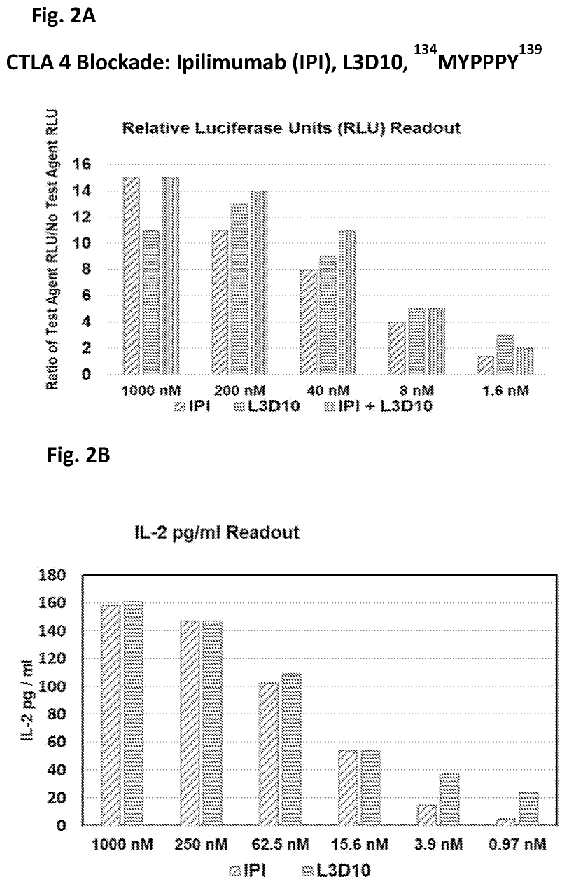

FIG. 2A and FIG. 2B show the typical dose response curve for standard conventional anti-CTLA 4 antibodies titration in the Promega CTLA 4 functional blockade assay: Ipilimumab (Selleckem cat no A2001) and L3D10 (May et al ‘05) [and JA1020 (Promega) data not shown] as assessed by luciferase activity and by IL-2 protein expressed.

The luciferase activity was reported in the graph in FIG. 2A as the ratio of the test agent's relative luciferase units divided by the no agent (background) luciferase activity. This gives a value that can be compared between samples assessed in different assays. The maximum ratio, the maximum activity, is seen at antibody concentrations >40 nM and decreases as expected with the decreasing antibody concentration. Note that the two antibodies Ipilimumab and L3D10 when combined in a mixture also yield a titration response where the maximal activity luciferase ratio is about 14. For a titration curve of Ipilimumab tested starting at 4000 nM, the curve shape was similar to that seen here and the maximal response was 12-18 range, with a plateau in the curve from 4000 nM to 200 nM.

FIG. 2B shows a graph that is a measure of IL-2 expressed by Jurkat cells with increasing anti-CTLA 4 concentration. This set of data was from a different assay than the luciferase readout. The amount of IL-2 expressed decreases with decreasing antibody concentration and matches the graph depicting the luciferase readout. The luciferase readout is a valid surrogate for activated Jurkat cell IL-2 response to CTLA 4 blockade and signaling.

The activation of the Jurkat cell in this αCTLA 4 functional blockade assay is strictly dependent on the presence of Raji cells. In the absence of Raji cells, Ipilimumab yields RLU that is <1% of the luciferase activity observed when Raji cells are present.

### CTLA 4 and PD1 Functional Blockade Assay

Both CTLA 4 and PD1 signaling pathways converge to dampen CD28 driven T cell activation. Thus, a functional assay for both signaling pathways may be a better representation of the T cell activation process in vivo.

Promega has developed a reliable easy to use kit form of the αCTLA 4 and αPD1 functional blockade assay (CS1978D04 and CS1978D08). Specifically, Jurkat cells have been engineered for heterologous expression of CTLA4 and PD1 with an IL-2 promoter NF-κB NFAT-driven luciferase readout. Raji cells, which natively express CD80, were engineered to co-express anti-CD3 and PDL1.

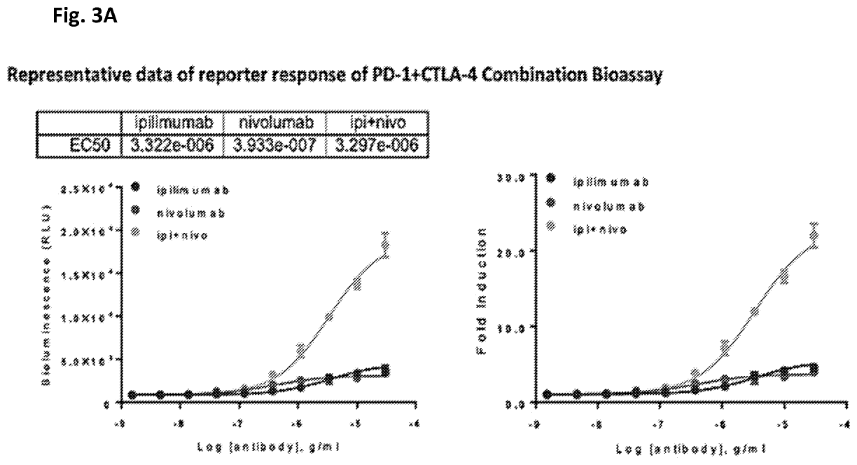

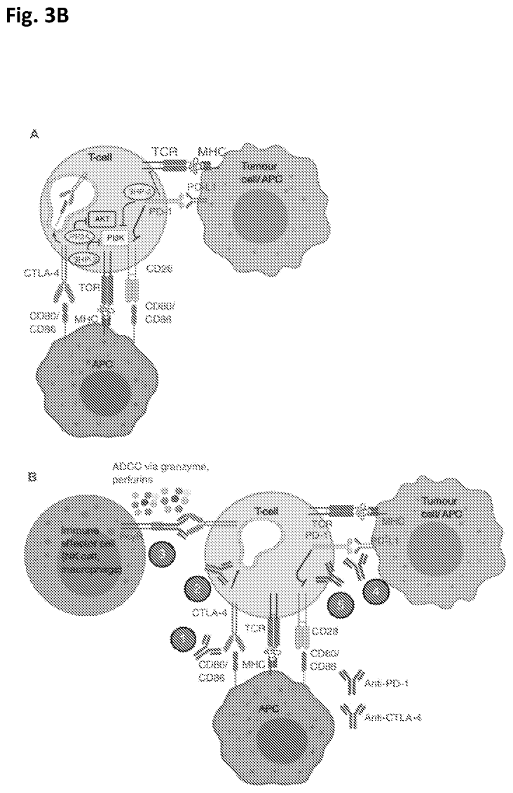

FIG. 3, adapted from Promega's assay manual, is a graph of titration curve αCTLA 4 antibody Ipilimumab and αPD1 antibody Nivolumab as single agents and in combination. Note the marginal response of the single agents plateauing at about 3× stimulation above background while when used in combination the luciferase response is 20× above background and 6× either agent alone. This synergistic activation of Jurkat T cells by blocking both CTLA4 and PD1 results from the fact that either CTLA4 pathway or PD1 pathway can block T cells independently of each other. Only upon blocking both pathways, can the T cell activation be suppressed more completely.

Accordingly, this assay monitors the ability of anti-CTLA4 antibodies to block T cell activation in the presence of the active PD1 pathway, or in combination with an anti-PD1 antibody. Both PD1 and CTLA4 signaling pathways converge on IL-2 expression.

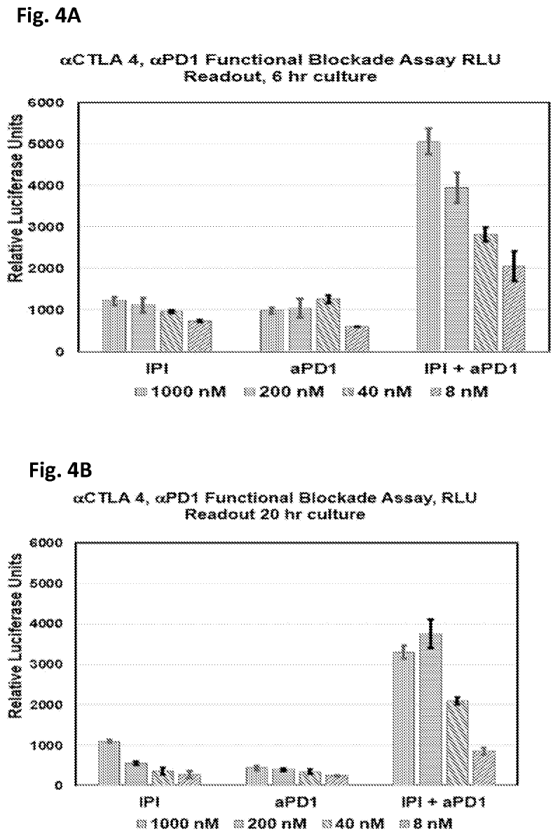

We have found similar results when testing our reagents Ipilimumab, αPD-1 (BioLegends) and the mixture of Ipilimumab+αPD1 (FIG. 4A and FIG. 4B). The titration curve is from samples done in triplicate. The error bars shown are standard deviations in the relative luciferase units. The concentrations shown in the mixture are the concentrations of each component thereby yielding a total concentration of antibodies that is twice that listed. FIG. 4 shows two assay incubation times for two identical assay plates; the first plate was read after 6 hr (FIG. 4A) and the second plate was read at 20 hr (FIG. 4B). The luciferase enzyme used by Promega is engineered to have a short half-life with the aim to reveal the “instantaneous” level of gene expression at the assay time. There is no significant accumulation of luciferase enzyme.

### CytoStim™/LPS Primary Human T Cell Activation Assay

The ex vivo activity of checkpoint inhibitors on native human T cell activation was tested after stimulation with CytoStim™ (Milteni Biotec, 1:400 dilution) and lipopolysaccharide (LPS, 100 ng/ml). CytoStim™ is an antibody-based reagent that acts similarly to a super-antigen but independently of certain VB domains of the T cell receptor (TCR). It causes activation of T cells.

### Example 2: a Mouse Anti-CTLA4 Antibody, BNI3, Induced Activation of Primary Human T Cell

Frozen packs of Human PBMCs were obtained with institutional consent from qualified donors, thawed and cultured in X-vivo cell media+5% heat-inactivated fetal bovine serum (HI FBS) at 2×10⁷ cells per ml overnight and aliquoted into microtiter wells at 75 ul per well. 3 ul of CytoStim™ (3 × concentrate) and 50 ul of LPS (3 ug per ml) were added. 125 μl of test agent made up in X-vivo+5% FBS was added. Assays were incubated at 36° C./5% CO2 for 24, 48 or 72 hours. The supernatants were recovered and assayed for IL-2.

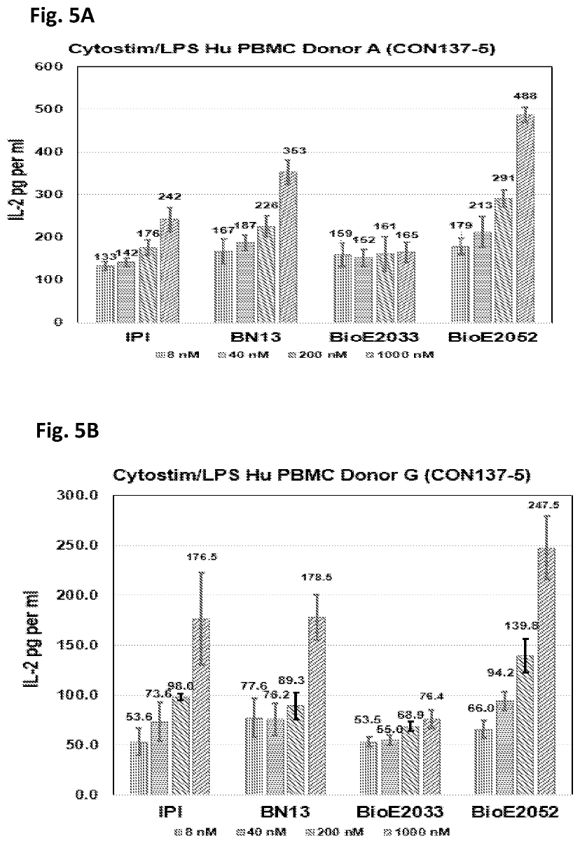

FIG. 5 shows the representative results from 2 donors. The ipilimumab was sourced from Selleckem, BN13 is mouse BNI3 from BioXcell (BN13 is the same antibody as BNI3 (Castan et al (1997) Immunology, 90, 265-271), but for cataloguing at BioXcell it is listed as BN13 not BNI3) and BioE2052 is an anti-CTLA4 diabody (see Table 7). In 3 donors A, G and F(data not shown), BN13 was 1-1.5 × as active as Ipilimumab in inducing IL-2 expression while BioE2052 was 1.5 × as active as Ipilimumab in donor G but 2 × as active in donors A and F(data not shown). In addition, in a repeat assay with donors A, E and F, BioE2052 was also twice as active as Ipilimumab in inducing IL-2 expression.

An important interpretation from the CytoStim™/LPS Human PBMC assay is the comparison of its results to the activity determined using (i) Promega anti-CTLA 4, and (ii) anti-CTLA 4+anti-PD1 functional blockade assays. In both assays BNI3 was <60% of the biological activity of Ipilimumab throughout the titration curve. Yet in the CytoStim™/LPS assay with primary Human PBMC, the activity titration curves for BNI3 and Ipilimumab were equivalent.

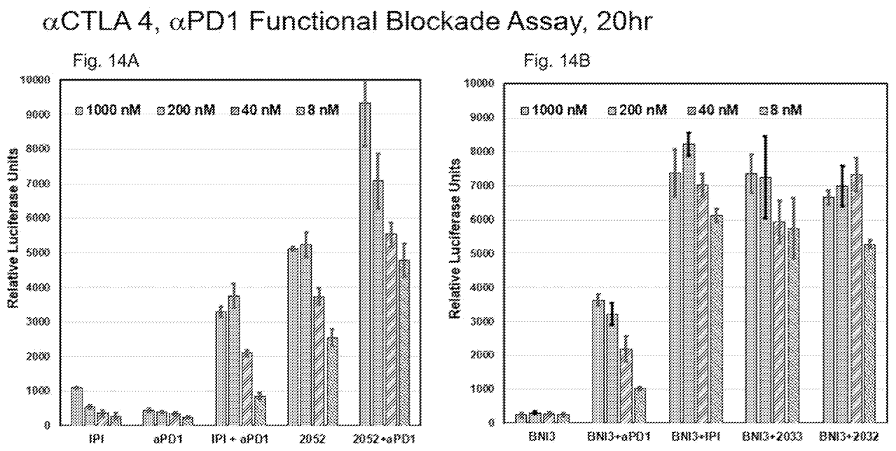

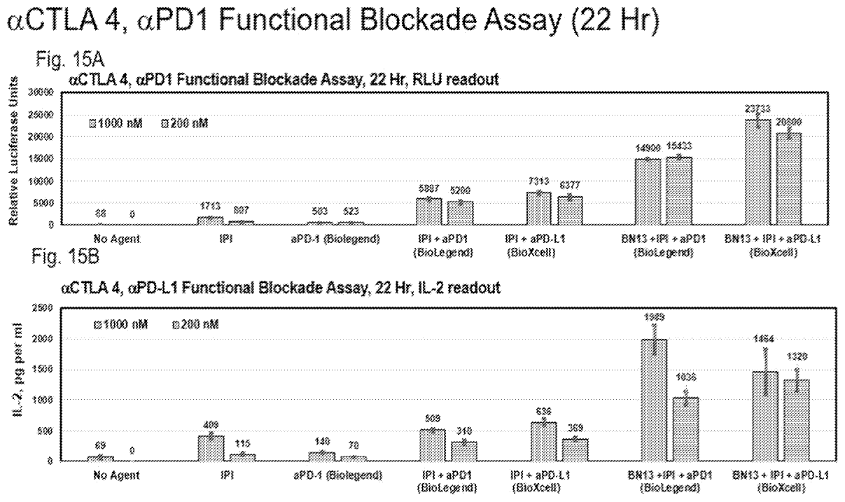

Interestingly, the response of BioE2052 in the anti-CTLA4 anti-PD1 assay is more complex. BioE2052 showed consistently a higher magnitude of response in both the anti-CTLA4 functional blockade and the primary Human PBMC assay (FIG. 5A and FIG. 5B). However, in the CTLA4 and PD1 assays, early in the assay incubation time (6 hr) the response of Ipilimumab, anti-PD1, and BioE2052 are equivalent (FIG. 13A). But after a longer 20 hr incubation, BioE2052 has significantly more luciferase activity (FIG. 14A). The differential and dynamic responses of the same anti-CTLA4 binders in different biological assays that are representative of different states of immune cells supports a hypothesis that modifiers of immune cell action could be selective and specific. There are likely specific therapeutic applications for different anti-CTLA4 targeted antibodies.

### Example 3: The Humanization of BNI3 Antibody

BNI3 is a mouse IgG2a described in Castan et al (1997) Immunology, 90, 265-271. BNI3 is purified from a hybridoma and available commercially from numerous reagent suppliers. BNI3 was sourced from BioXcell which is listed as BN13 in their catalog. BNI3 amino acid sequence is not known in the art.

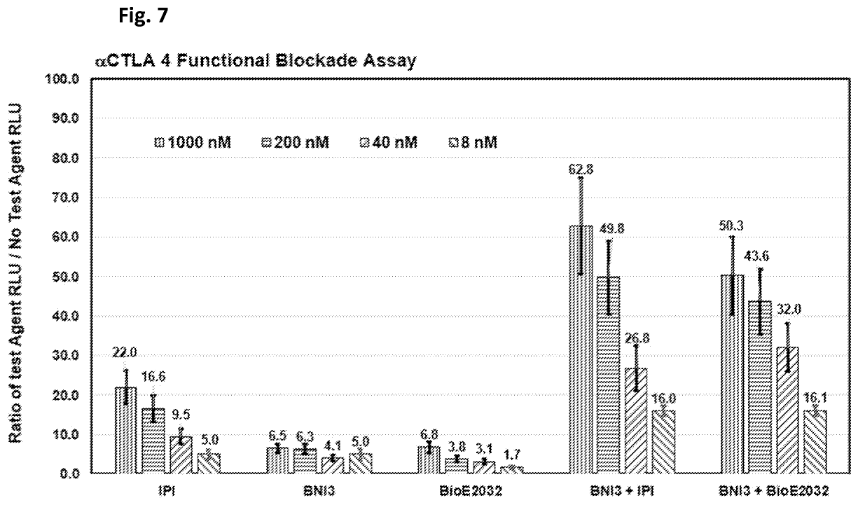

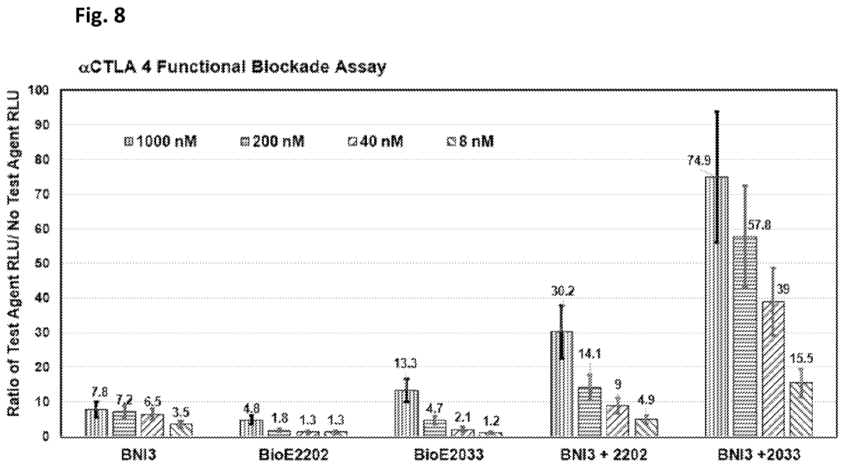

As shown above, in the CytoStim™/LPS assay with primary Human PBMC, the activity titration curves for BNI3 and Ipilimumab were equivalent (Example 2 and FIG. 5). However, the combination of BNI3 and Ipilimumab showed surprising synergism. Specifically, BNI3 was found in combinations with IPI (full length of Fab2) or 121 (full length or Fab2) to be 3-4 × more potent than IPI in the Promega anti-CTLA4 and Promega anti-CTLA4, anti-PD1 functional blockade assays (FIG. 7 and FIG. 8; see also below). To advance these combination antibodies for potential therapeutic applications; BNI3 was humanized.

The humanization process occurred through three stages:
I. Determination of the amino acid sequence of BNI3, the mouse antibody.
II. The humanization of BNI3, specifically engineering a human antibody to have BNI3 binding characteristics.
III. Identifying a humanized BNI3 variant that has the same or enhanced functional characteristics as compared to the mouse BNI3 (e.g., in the functional assays described above).

### Determination of the Amino Acid Sequence of Mouse BN13

Amino acid determination:

1. Fifteen digestions were prepared using six different enzymes (Pepsin, Trypsin, Chymotrypsin, Asp N, Lys C, Nonspecific).

2. The digestions for the sample were processed with disulfide reduction, alkylation, and then enzyme digestion. Each digestion contains peptides from all immunoglobulin chains.

3. Digestions were analyzed by LC-MS/MS using a Thermo-Fisher Orbitrap Fusion™ mass spectrometer.

4. Peptides were characterized from LC-MS/MS data using de novo peptide sequencing and then assembled into antibody sequences.

### SUMMARY

Host Species: Mus musculus

### Isotype: Heavy Chain—Gamma 2A, Light Chain—Kappa

Sequence of mouse BNI3:

>Light chain [214 AA]
DIVMTQSQKFMSTSVGDRVSVTCKASQNVGTYVAWYEQKLGQSPKALIF
SASYRYTGVPDRFTGSGSGTDFTLTISNVQSEDLAEYFCQQYDSYPLTF
GGGTKLEIKRADAAPTVSIFPPSSEQLTSGGASVVCFLNNFYPKDINVK
WKIDGSERQNGVLNSWTDQDSKDSTYSMSSTLTLTKDEYERHNSYTCEA
THKTSTSPIVKSFNRNEC
Heavy chain [449 AA]
DVQLQESGPGLVKPSQSLSLTCSVTGYSITSGYVWNWIRQFPGNKLEWM
GYISHDGNTNYNPSLKNRISITRDTSKNQFFLKLNSVTTEDSATYYCTR
NYGYGGTMDYWGQGTAVSVSSAKTTAPSVYPLAPVCGDTTGSSVTLGCL
VKGYFPEPVTLTWNSGSLSSGVHTFPAVLQSDLYTLSSSVTVTSSTWPS
QSITCNVAHPASSTKVDKKIEPRGPTIKPCPPCKCPAPNLLGGPSVFIF
PPKIKDVLMISLSPIVTCVVVDVSEDDPDVQISWFVNNVEVHTAQTQTH
REDYNSTLRVVSALPIQHQDWMSGKEFKCKVNNKDLPAPIERTISKPKG
SVRAPQVYVLPPPEEEMTKKQVTLTCMVTDFMPEDIYVEWTNNGKTELN
YKNTEPVLDSDGSYFMYSKLRVEKKNWVERNSYSCSVVHEGLHNHHTTK
SFSRTPGK

### Humanization of Mouse BNI3

The VH and VL sequences were analyzed through the IGMT Gap Align program against all known antibody germline sequences. CDR regions were assigned using the IMGT definition. The sequence was most closely aligned to human germline sequences, specifically the IGHV4-38-2*02 family for the VH and IGKV1-8*01 for the V_{L}. A total of 4 humanized heavy chains and 4 humanized light chains were designed. Each of these was synthesized individually and cloned into human IgG1 heavy chain and human kappa light chain expression vectors, respectively. At the point of transfection, all possible combinations of the humanized sequences were made to create a total of 16 different humanized antibodies. Table 5 summarizes the constructs that were prepared. The original mouse BNI3 was rendered into a clone identified as cAb7125-1.1, a chimeric form of this was made with Human IgG1 Fc sequences, cAb7125-10.0 and the 16 variants comprising of 4 V_{H} chains paired with 4 VL chains to generate the series cAb7126-10.0 to cAb7141-10.0.

TABLE 5
Summary of the constructs comprising the humanized BNI3

| ID | Clone name | Format | VH | VL |
|---|---|---|---|---|
| cAb7125-1.1 | BNI3 | Mouse IgG1 | cAb7125-VH | cAb7125-VL |
| cAb7125-10.0 | BNI3 | Chimeric human IgG1 | cAb7125-VH | cAb7125-VL |
| cAb7126-10.0 | humBNI3-v1 | Humanized human IgG1 | cAb7126-VH | cAb7126-VL |
| cAb7127-10.0 | humBNI3-v2 | Humanized human IgG1 | cAb7126-VH | cAb7127-VL |
| cAb7128-10.0 | humBNI3-v3 | Humanized human IgG1 | cAb7126-VH | cAb7128-VL |
| cAb7129-10.0 | humBNI3-v4 | Humanized human IgG1 | cAb7126-VH | cAb7129-VL |
| cAb7130-10.0 | humBNI3-v5 | Humanized human IgG1 | cAb7130-VH | cAb7126-VL |
| cAb7131-10.0 | humBNI3-v6 | Humanized human IgG1 | cAb7130-VH | cAb7127-VL |
| cAb7132-10.0 | humBNI3-v7 | Humanized human IgG1 | cAb7130-VH | cAb7128-VL |
| cAb7133-10.0 | humBNI3-v8 | Humanized human IgG1 | cAb7130-VH | cAb7129-VL |
| cAb7134-10.0 | humBNI3-v9 | Humanized human IgG1 | cAb7134-VH | cAb7126-VL |
| cAb7135-10.0 | humBNI3-v10 | Humanized human IgG1 | cAb7134-VH | cAb7127-VL |
| cAb7136-10.0 | humBNI3-v11 | Humanized human IgG1 | cAb7134-VH | cAb7128-VL |
| cAb7137-10.0 | humBNI3-v12 | Humanized human IgG1 | cAb7134-VH | cAb7129-VL |
| cAb7138-10.0 | humBNI3-v13 | Humanized human IgG1 | cAb7138-VH | cAb7126-VL |
| cAb7139-10.0 | humBNI3-v14 | Humanized human IgG1 | cAb7138-VH | cAb7127-VL |
| cAb7140-10.0 | humBNI3-v15 | Humanized human IgG1 | cAb7138-VH | cAb7128-VL |
| cAb7141-10.0 | humBNI3-v16 | Humanized human IgG1 | cAb7138-VH | cAb7129-VL |

The nucleic acid comprising the sequences encoding V_{H} and V_{L} of each antibody was transfected into HEK 293 cells. After 6 days of culture, the culture media was harvested and recombinant antibodies were isolated by Protein A affinity chromatography. Eluted proteins were exchanged into PBS and analyzed by SDS-PAGE (sodium dodecyl sulphate polyacrylamide gel electrophoresis) and HPLC (high performance liquid chromatography).

SDS-PAGE and SEC HPLC showed that most of the constructs expressed well and were found to be >90% homogenous as single bands on gels and a single major peak on SEC.

The sequences of the VH and VL domains of the humanized BNI3 antibodies are presented in Table 4.

### Example 4: Identifying a Humanized BNI3 Variant with Similar or Enhanced Ability to Activate T Cells

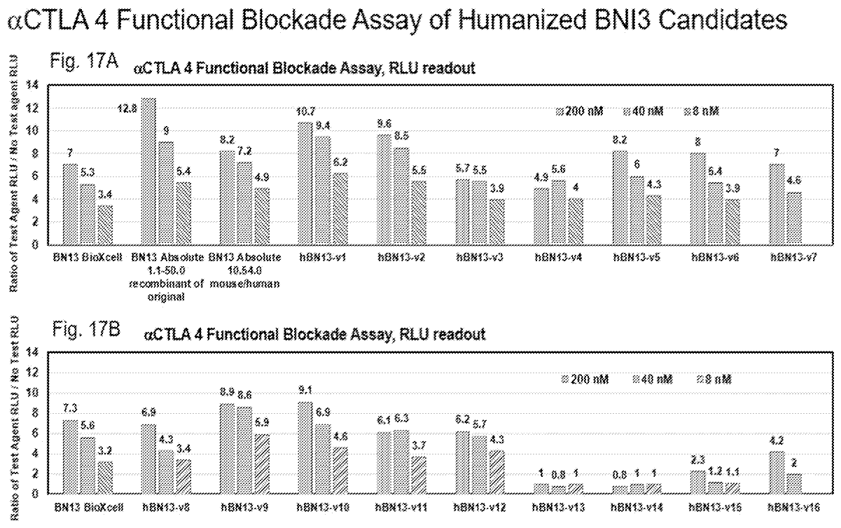

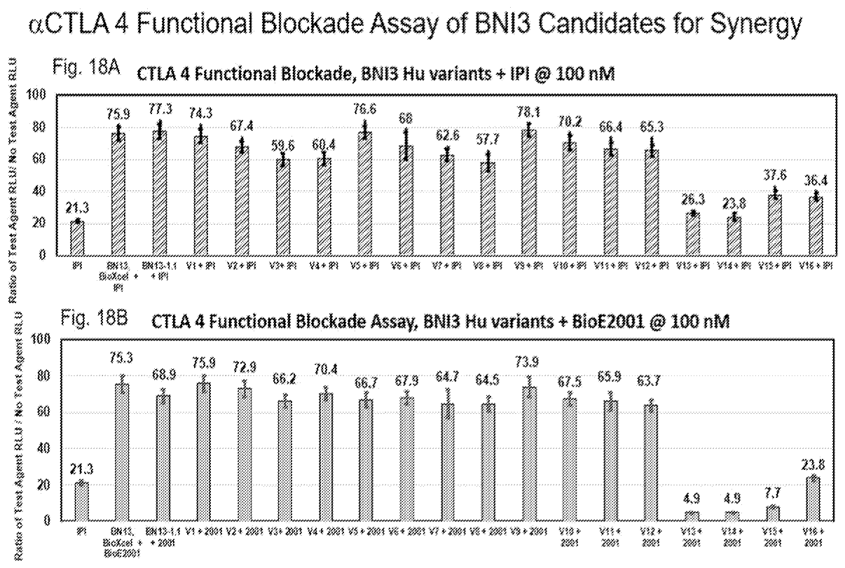

The Promega αCTLA4 functional blockade assay was used to identify the humanized variant of mouse BNI3 that is most active in blocking the CTLA4 signaling pathway and consequently activating T cells. The humanized variants of BNI3 (referred to as hBNI3, HumBNI3, or HuBNI3; hBNI3-v1 to hBNI3-v16) were assayed in the Promega CTLA 4 functional blockade assays under standard conditions (FIG. 17A and FIG. 17B). The variants were assayed in titration (by protein concentration) of 200 nM, 40 nM, and 8 nM in duplicates. The measured luciferase activity was then processed to yield a ratio of relative luciferase units over background luciferase activity. The recombinant form of mouse BNI3 and ipilimumab were used as standards. Typically, in this assay Ipilimumab had a score of 15+/−2. The observed activity for mouse BNI3 from BioXcell was as expected based on the previous observation. Surprisingly, the recombinant expressed form of hBNI3 had a higher activity than mouse BNI3 in 3 different assays. The notably more active variants were hBNI3-v1,-v2,-v9 and v10.

The human BNI3 variants (hBNI3s) were next tested for their ability to synergize with ipilimumab (IPI) or with BioE2001.

Each of human BNI3 variants was combined with IPI to yield a final concentration of 100 nM each (FIG. 18A). Variants hBNI3-V1 to hBNI3-V12 showed good synergy with IPI, while variants hBNI3-V13, hBNI3-V14, hBNI3-V15 and hBNI3-V16 revealed an additive response with IPI.

Each of human BNI3 variants was combined with BioE2001 (FIG. 18B). BioE2001 is the mouse antibody Mab 26 (also referred to herein as the mouse 121 antibody) described in patent U.S. Pat. No. 7,034,121B2 (which is incorporated herein by reference). BioE2001 was further engineered to have a LALA double mutation in the Fc region. The LALA double mutation (Leu234Ala together with Leu235Ala) in the Fc region diminishes the effector functions of the antibody (Lund et al. (1992) Mol. Inununol . . . 29, 53-59). BioE2001

The hBNI3-V1 to hBNI3-V16 variants were tested in combination with BioE2001 (FIG. 18B). The hBNI3-V1 to hBNI3-V12 showed good synergy with BioE2001. Unexpectedly, hBNI3-V13+BioE2001, hBNI3-V14+BioE2001 and hBNI3-V15+BioE2001 were only marginally active. Generally, the variants that were relatively inactive as single agents in the functional blockade assay were also inactive in combination with either IPI or BioE2001.

The CTLA 4 functional blockade assays of the humanized BNI3 variants, hBNI3-V1 to hBNI3-V16, alone or in combination with IPI or BioE2001 provided a means to identify the most active humanized form of themouse BNI3. A number of candidate variants advanced into development from which hBNI3-VI and hBNI3-V2 were selected for further development.

### Example 5: Development of Single Action Antibodies, Antibody Fc Relevance to Anti-CTLA4 In Vitro and In Vivo Biological Activities

In designing anti-CTLA4 antibodies for therapeutic application there is consideration to the final structure of the antibody even when the design is a conventional IgG format. For anti-CTLA4 antibodies, the Fc structure affects its biological activity and toxicity (Bauche et al ‘20). The Fc, in addition to the usual pharmacokinetic role binding to FcRn, appears to have at least 3 other functions roles: (1) directing TREG depletion through ADCC (Simpson et al ‘13, Selby et al ‘13 Ha et al ‘19, Vargas et al ‘18); (2) “bridging” to accessory immune cells to affect immune cell activation (Waight et al ‘18); and (3) a contributing role in gastro intestinal inflammatory toxicity (Bauche et al ‘20).

There have been two pioneering anti-CTLA 4 antibodies, Ipilimumab an IgG1 and Tremelimumab an IgG2, both of which have undergone the full clinical trial process. Only BMS's Ipilimumab has been approved (in 2011) while AstaZeneca's Tremelimumab has not been approved and remains in orphan drug status. Both antibodies by structural studies have been shown to bind similarly at the B7 ligand binding sites, 134MYPPPY139 but differ in their Fc domains and hence in some of their biological properties (He et al ‘17). Human IgG1 Fc directs ADCC while IgG2 does not. Ipilimumab can direct ADCC and in mouse models has shown its anti-tumor killing efficacy to be positively correlated with its binding efficacy to FcγRIII and hence TREG depletion efficacy. Tighter binding leads to more effective TREG depletion in mouse models and more effective tumor cell killing (Vargas et al ‘18). However, in humans it has been unclear how Ipilimumab and Tremelimumab actually work. Sharma et al ‘18 could find no evidence of TREG depletion in Humans treated with Ipilimumab for melanoma, prostate cancer or bladder cancer, though possibly this is a matter of sample timing (Quezada & Peggs ‘18). The lack of evidence for TREG depletion is support for the hypothesis that anti-CTLA 4 antibody clinical efficacy could be solely due to blockade of CTLA 4 binding of B7 ligands. Though the current thinking is that CTLA may just fortuitously be a good marker for TREG depletion (Du et al ‘18, Stone et al ‘21), TREG depletion alone leads to tumor killing in mouse models. By contrast, recent work by Bauche etal ‘20, Schofield et al ‘20 and Sato et al ‘22 demonstrated that anti-CTLA 4 antibodies with no ADCC ability can lead to anti-tumor action in some mouse tumor models, indicating that modulating the CTLA4 signaling pathway alone (without TREG depletion) may be sufficient for anti-tumor activity.

The CTLA4-binding proteins or combinations thereof described herein utilize both Fc-plus and Fc-minus variants to allow balancing the efficacy and toxicity in different tumors or different tumoral microenvironments (even within the same tumor type).

The Fc-minus variants can be accomplished by removing all or portions of the Fc domain, or through engineering the Fc domain to include LALA mutation (Leu234Ala and Leu235Ala) or LALAPG mutation (L234A, L235A, and P329G). These mutations eliminate complement binding, fixation, FcγR binding and subsequent antibody dependent cell mediated cytotoxicity (ADCC) with human immune cells (Lund et al ‘91, Schlothauer et al. (2016) Protein Engineering, Design & Selection 10:457-466) or mice (Lo et al ‘17).

LALAPG Fc versions of hBNI3-V1, hBNI3-V2, Ipilimumab, and BioE2032 were constructed and tested for biological activity in the Promega anti-CTLA4 functional blockade assay. We and others have found that for conventional format anti-CTLA4 antibodies with Fcs that binding to FcγRII on the Raji cells affects activity. Anti-CD32 antibodies which block FcγRII binding reduced anti-CTLA4 blocking activities by 40-60% (present disclosure; data not shown), while increasing Fc binding to FcγRII binding enhanced anti-CTLA 4 functional blockade activity (Waight et al ‘20). We expected a decrease in functional activity when the Fc is mutated to LALAPG.

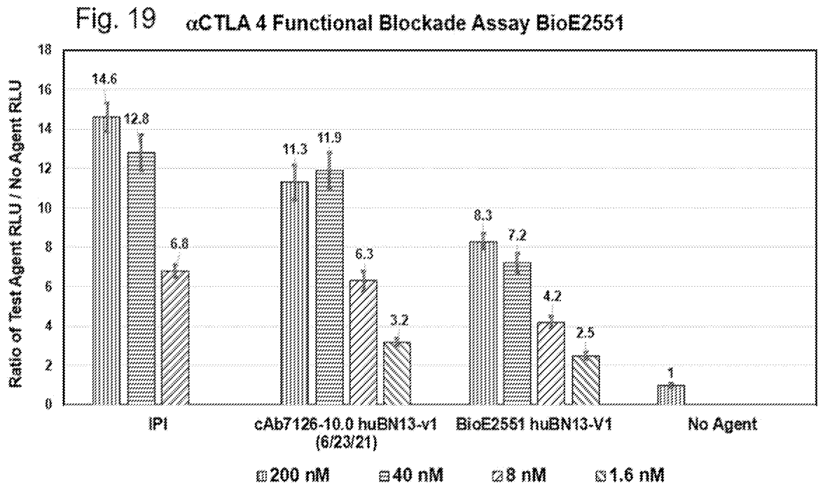

FIG. 19 depicts the biological response titration of BioE2551 which is a version of human BNI3-VI with LALAPG IgG1 Fc. The LALAPG Fc variant does not bind FcγRII Fc receptors on Raji cells. Note the ˜30% decrease in activity throughout the titration curve. The observed decrease was as expected.

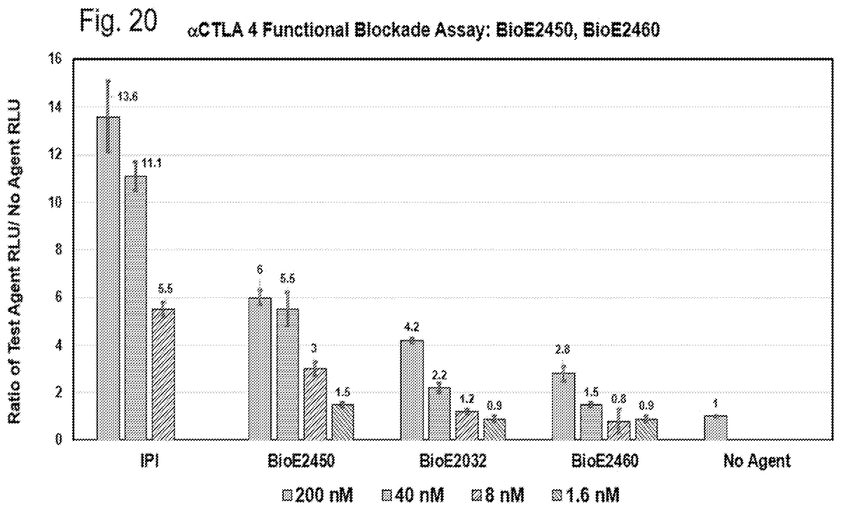

FIG. 20 depicts the biological response titration of BioE2450 which is variant of Ipilimumab but with IgG1 Fc LALAPG. Loss of FcγR binding resulted in a 60% decrease in the biological activity throughout the titration curve. BioE2460 is a variant of BioE2032 with IgG1 Fc LALAPG, and there was a 45% drop in biological activity as expected.

In the Promega anti-CTLA4 functional blockade assay, anti-CTLA4 antibodies with conventional Fcs bind FcγR on the Raji cell, and facilitate a “bridging” effect between the Jurkat readout cell and Raji cell as hypothesized by Waight et al ‘20. Such bridging effect is also evident in the in vitro antigen assays with anti-CTLA4. The mechanism by which this bridging effect contributes to the anti-CTLA4 blockade activity is unknown. The removal of FcγR binding (e.g., via LALAPG mutation) eliminates its effect on anti-CTLA4 blockade activity and leads to an overall decrease in the biological activity. However, at the same time, the removal of FcγR binding unveils the anti-CTLA4 blockade activity that is attributed strictly to their binding of specific epitopes on CTLA4, i.e., its effect on CTLA4 signaling pathway. This binding presumably leads to blocking the binding of CTLA4 to B7, and subsequently enhances CD28 activation by the now available B7.

### Example 6: Unexpected Synergy by a Combination of Anti-CTLA4 Antibodies

It is discovered herein that some combinations of antigen-binding proteins (e.g., antibodies) that target multiple epitopes on CTLA4 resulted in an unexpected synergy in the anti-CTLA4 blockade activity, as determined by e.g., IL-2 activation or IL-2 promoter drive luciferase expression in the αCTLA4 blockade assay.

Specifically, it was found that some of the multiple epitopes on CTLA4 have different “signaling pathways” (or processes) to IL-2 gene activation (luciferase expression) such that binding to 2 different epitopes with two different antibodies led to a summation of the signals that is reflected in the IL-2 expression. This summation for some combinations of antibodies was additive, less than additive, or synergistic.

This synergistic property is evident in combinations of HuBNI3+Ipilimumab, as well as in combinations of HuBNI3+BioE2032. However these antibodies have the ability to bind FcγR and by a potential bridging effect to immune accessory cells that contribute to the anti-CTLA4 blockade activities. To determine whether the combined effect of these antibodies is solely due to their effects on the CTLA4 signaling activity, we have engineered these antibodies to have Fc with LALAPG mutation (L234A, L235A, and P329G) which effectively eliminate the FcγR binding and subsequent ADCC and complement fixation.

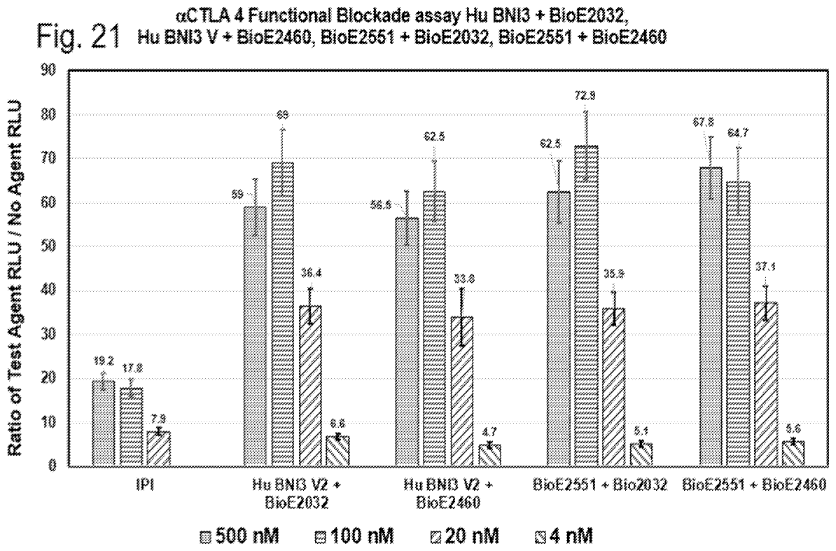

FIG. 21 describes titration curves of IPI, HuBNI3-V2+BioE2032, Hu BNI3 V2+BioE2460, BioE2551+BioE2032, and BioE2551+BioE2460 in the anti-CTLA 4 functional blockade assay. The combinations of antibodies exhibited the 3.5× increase in activity compared to Ipilimumab throughout their titration curves. More interestingly the Fc L234A, L235A P329G variants revealed the same degree of activity as their parental forms. Eliminating FcγR binding did not affect the activity of the antibody combinations of BioE2551+BioE2460.

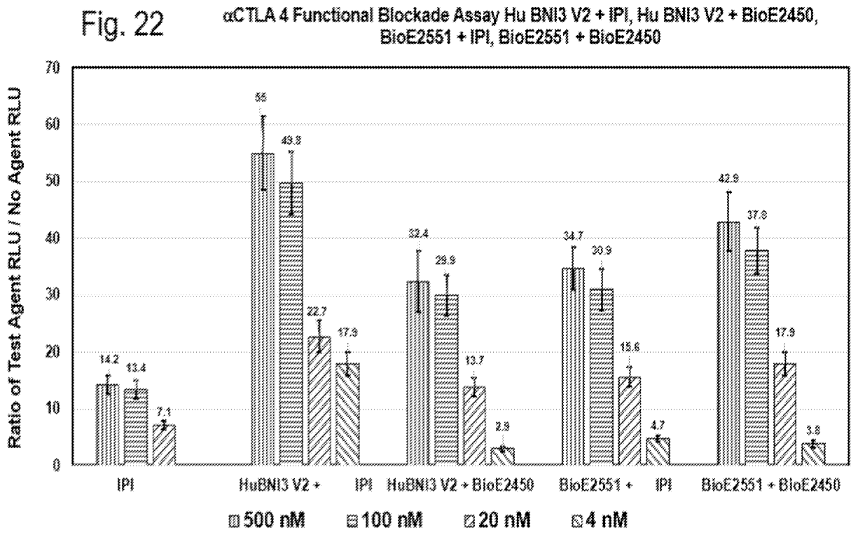

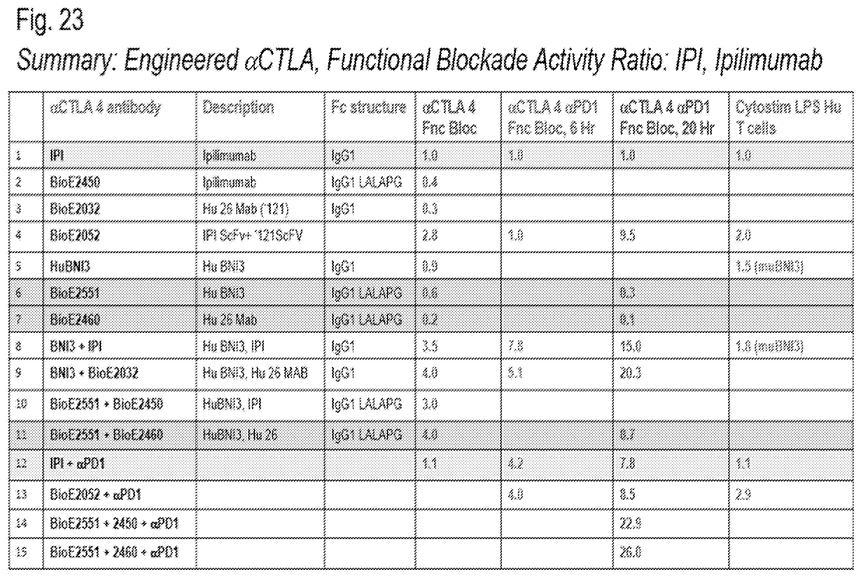

FIG. 22 describes titration curves of IPI, Hu BNI3 V 2+Ipilimumab, Hu BNI3 V2+BioE2450, BioE2551+Ipilimumab, and BioE2551+BioE2450 in the anti-CTLA4 functional blockade assay. The combinations of antibodies exhibited the 2-3× increase in activity compared to Ipilimumab throughout their titration curves. More interestingly the Fc L234A, L235A P329G variants revealed that said combinations have a similar degree of synergistic activity as their parental forms. BioE2551+BioE2450 was 3× as active as ipilimumab, consistent with the activities seen for huBNI3+Ipilimumab.

### Example 7: Identifying Highly Active Combinations of Anti-CTLA 4 Antibodies

The antibodies listed in Table 6 were either provided herein (e.g., those designated as from BioEntre LLC), or purchased from commercial sources. The antibodies were tested in triplicates in a titration curve of 1000, 200, 40 and 8 nM as single agents or in combinations with each other. A standard deviation for each concentration was determined. In general, the standard deviation was less than +/−15%. FIG. 7 and FIG. 8 are illustrative examples of such assay results.

TABLE 6
αCTLA 4 antibodies used in the combinatorial format
for the αCTLA 4 Functional Blockade Assay

| | αCTLA 4 Antigen- | |
|---|---|---|
| | binding proteins | Source |
| 1 | Ipilimumab | Selleckchem, cat. No. A2001 |
| 2 | IPI Fab2 (BioE2022) | BioEntre LLC, synthetic Fab2 with |
| | | Absolute AB dimerization technology. |
| 3 | IPI Fab (BioE2201) | BioEntre LLC |
| 4 | IPI ScFv | BioEntre LLC |
| 5 | 121 HuIgG1 | BioEntre LLC |
| | (BioE2032) | |
| 6 | 121 Fab2 (BioE2033) | BioEntre LLC, synthetic Fab2 with |
| | | Absolute AB dimerization technology. |
| 7 | 121 Fab (BioE2202) | BioEntre LLC |
| 8 | 121 ScFv | BioEntre LLC |
| 9 | BioE2052 | BioEntre LLC |
| 10 | L3D10 | BioLegend, GoInVivo purified anti-hu |
| | | CD152 (CTLA 4) antibody |
| 11 | JA1020 | Promega JA 1020, control anti-Hu CTLA4 |
| | | antibody |
| 12 | BNI3 | BioXcell, BN13, inVivoMab, anti-human |
| | | CTLA4 (CD152) mouse IgG2a |
| 13 | Mab325 | Mouse IgG2b, Biotechne, R & D Systems |
| 14 | Mab386 | Mouse IgG1, Biotechne, R & D Systems |
| 15 | Mab3251 | Mouse IgG2b, Biotechne, R & D Systems |
| 16 | Mab3252 | Rabbit IgG, Biotechne, R & D Systems |
| 17 | Mab3253 | Rabbit IgG, Biotechne, R & D Systems |
| 18 | Mab3254 | Mouse IG1, Biotechne, R & D Systems |
| 19 | ANC152 | Ancell, cat. No 359-020 |

TABLE 7
Exemplary antigen-binding proteins that bind CTLA4

| Name | Construct |
|---|---|
| BioE2001 | Mouse Mab 26 (also referred to as 121 antibody) with the |
| | LALA mutation. |
| BioE2012 | Half antibody-ScFv |
| | IPI Fab Fc 121 ScFv [VL121 (G4S)3 VH 121] |
| BioE2021 | Ipi scFv |
| | [VL Ipi]-(Gly4Ser)3-[VH Ipi]-His10 tag |
| BioE2022 | Ipi F(ab′)2. |
| | BioE2022 also refers to the VH and CH1 domains of ipi |
| | that form ipi F(ab′)2 with BioE2023. |
| BioE2023 | Ipi VL and CL domains |
| BioE2031 | 121 scFv |
| | [VL 121]-(Gly4Ser)3-[VH 121]-His10 tag |
| BioE2032 | 121 HuIgG1 |
| BioE2033 | 121 F(ab′)2. |
| | BioE2033 also refers to the VH and CH1 domains of |
| | 121 that form 121 F(ab′)2 with BioE2034. |
| BioE2034 | 121 VL and CL domains |
| BioE2041 | Tandem Biparatopic ScFv (TBS); self-dimerizes |
| | VLIPI (G4S)3 VHIPI (KEA)6 VL121 (G4S)3 VH121 |
| | His6 tag |
| BioE2042 | Tandem Biparatopic ScFv (TBS); self-dimerizes |
| | VL121 (G4S)3 VH 121 (KEA)6 VLIPI (G4S)3 VH IPI |
| | His6 tag |
| BioE2051 | Anti-CTLA4 diabody (single-chain diabody; self- |
| | dimerizes; Tandem Diabody (TandAb)) comprising: |
| | [VH 121]-(GlyGlySer)3-[VL Ipi]-(GlyGlySer)3- |
| | [VH Ipi]-(GlyGlySer)3-[VL121]-His10 Tag |
| | ( |
| BioE2052 | Anti-CTLA4 diabody (single-chain diabody; self- |
| | dimerizes; Tandem Diabody (TandAb)) comprising: |
| | [VH Ipi]-(GlyGlySer)3-[VL 121]-(GlyGlySer)3- |
| | [VH 121]-(GlyGlySer)3-[VL Ipi]-His10 Tag |
| | ( |
| BioE2061 | Tandem Fab |
| | VH121-CH-(G4S)3-VH IPI-CH-His tag (VL 121-CL/VL |
| | IPI-CL) |
| | BioE2061 comprises 3 polypeptides with the sequence of |
| | SEQ ID Nos: 31-33. See Wu et al. (2015) Mabs 7: 470-82 |
| BioE2062 | Tandem Fab |
| | VH IPI-CH-(G4S)3-VH 121-CH-His tag (VL IPI-CL/VL |
| | 121-CL) |
| | BioE2062 comprises 3 polypeptides with the sequence of |
| | SEQ ID Nos: 34-36. See Wu et al. (2015) Mabs 7: 470-82 |
| BioE2081 | Heterodimeric Fab/ScFv-Fc |
| | 121ScFv Fc IPI Fab Fc (Fc silent) KiH Fc |
| | BioE2081 comprises 3 polypeptides with the sequence |
| | of SEQ ID Nos: 47-49. BioE2081 contains the Fc-silent |
| | LALAPG mutation. |
| | *KiH stands for Knob in Hole. See e.g., Ridgway et al. |
| | (1996) Protein Engineering 9: 617-621 |
| BioE2082 | Heterodimeric Fab/ScFv-Fc |
| | 121 Fab Fc IPI ScFv Fc (Fc silent) KiH Fc |
| | BioE2082 comprises 3 polypeptides with the sequence |
| | of SEQ ID Nos: 50-52. BioE2082 contains the Fc-silent |
| | LALAPG mutation. |
| | *KiH stands for Knob in Hole. See e.g., Ridgway et al. |
| | (1996) Protein Engineering 9: 617-621 |
| BioE2091 | Tandem Fab |
| | VL IPI CL (G4S)3 VH 121 CH1 His tag (VH IPI CH1/VL |
| | 121 CL) BioE2091 comprises 3 polypeptides with the |
| | sequence of SEQ ID Nos: 37-39. See Wu et al. (2015) |
| | Mabs 7: 470-82 |
| BioE2092 | Tandem Fab |
| | VL 121 CL (G4S)3 VH IPI CH1 His tag (VH 121 CH1/VL |
| | IPI CL) |
| | BioE2092 comprises 3 polypeptides with the sequence of |
| | SEQ ID Nos: 40-42. See Wu et al. (2015) Mabs 7: 470-82 |
| BioE2111 | DART |
| | BioE2111 comprises 2 polypeptides with the sequence |
| | of SEQ ID Nos: 43-44. |
| BioE2201 | Ipi Fab′. BioE2201 also refers to the VH and CH1 |
| | domains of ipi that form ipi Fab′ with BioE2023. |
| BioE2202 | 121 Fab′. BioE2202 also refers to the VH and CH1 |
| | domains of 121 that form 121 Fab′ with BioE2034. |
| BioE2121 | Heterodimeric scFvs |
| | IPI scFv-Fc/121 scFv-Fc (KiH) |
| | BioE2121 comprises 2 polypeptides with the sequence |
| | of SEQ ID Nos: 53-54. BioE2121 contains the Fc- |
| | silent LALAPG mutation. |
| | *KiH stands for Knob in Hole. See e.g., Ridgway et al. |
| | (1996) Protein Engineering 9: 617-621 |
| BioE2400 | BNI3 mouse BNI3 |
| BioE2401 | BNI3 mouse human chimera BNI3 |
| BioE2410 | BioE2052 with IgG1 Fc (conventional Fc) |
| BioE2420 | BioE2052 with an Fc peptide fused to the carboxy |
| | terminus of BioE2052. |
| BioE2430 | BioE2052 with an albumin peptide fused to the carboxy |
| | terminus of BioE2052. |
| BioE2440 | Mouse BNI3 IgG2a with LALAPG mutation |
| BioE2450 | Ipilimumab IgGI with the LALAPG mutation |
| BioE2460 | Human 121 (also referred to as Mab 26) IgGI with the |
| | LALAPG mutation |
| BioE2501 | huBNI3-v1 |
| BioE2502 | huBNI3-v2 |
| BioE2503 | huBNI3-v3 |
| BioE2504 | huBNI3-v4 |
| BioE2505 | huBNI3-v5 |
| BioE2506 | huBNI3-v6 |
| BioE2507 | huBNI3-v7 |
| BioE2508 | huBNI3-v8 |
| BioE2509 | huBNI3-v9 |
| BioE2510 | huBNI3-v10 |
| BioE2511 | huBNI3-v11 |
| BioE2512 | huBNI3-v12 |
| BioE2513 | huBNI3-v13 |
| BioE2514 | huBNI3-v14 |
| BioE2515 | huBNI3-v15 |
| BioE2516 | huBNI3-v16 |
| BioE2551 | hBNI3-v1 IgG1 with the LALAPG mutation |
| BioE2552 | hBNI-v2 IgG1 with the LALAPG mutation |
| BioET1100 | BioE2551 |
| BioET1300 | BioE2460 |
| BioET1400 | BioE2552 + BioE2450 |
| BioET1500 | BioE2551 + BioE2460 |

*Ipi: ipilimumab (see U.S. Pat. No. 6,984,720 B1)
*121: the humanized antibody disclosed in U.S. Pat. No. 7,034,121 B2
*Included in Tables 6 and 7 are proteins that comprise or lack an Fc domain. For proteins that comprise an Fc domain, these Tables include those comprising either the wild-type Fc domain or any variation thereof, e.g., those comprising a mutation (e.g., LALA mutation, LALAPG mutation, or any equivalent mutation known in the art), truncation. Such variation may have reduced or no binding to one or more Fc receptors.

The activity curves for all the antibodies generally had half max activity concentrations in the 10-100 nM range and the curves would plateau at 200 nM and higher concentrations. The magnitudes of the αCTLA4 antibody activity curves, their plateaus, were antibody specific and varied with each antibody from a ratio RLU from 1 to 40. In this set of antibodies, Ipilimumab, L3D10, or JA1020 binds the B7 ligand binding site, while BNI3, or 121 antibodies do not bind the CTLA4 β7 ligand binding site. The antibodies Ipilimumab, L3D10, and JA1020 are in a conventional IgG format and have the similar maximal activity ˜15, while other antibodies are varied.

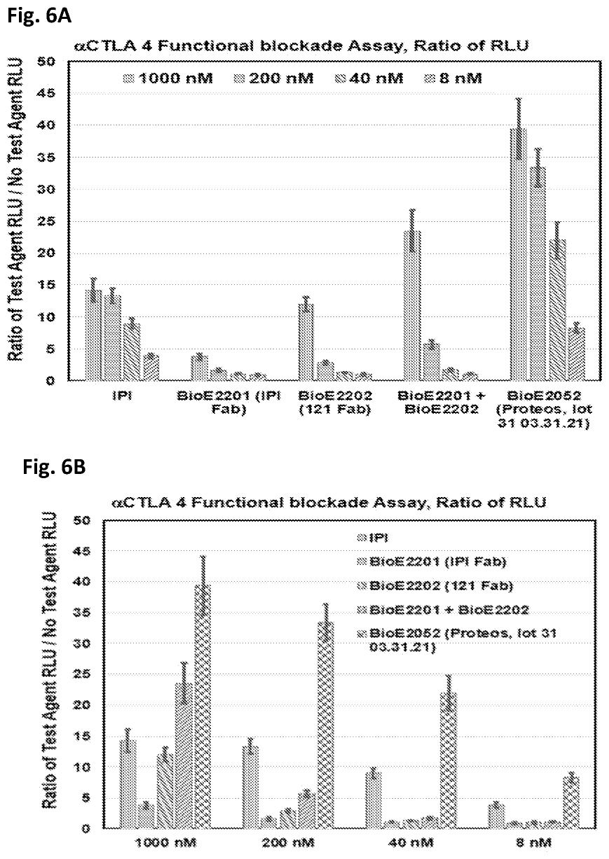

Interesting and relevant pairs of the CTLA4 antigen-binding proteins were repeated in at least two different assays. In FIG. 7, note the unexpected high level of activity, a synergistic level, arising from combinations of different antibodies. The pair of BNI3 and IPI was more than 3× more active than the higher activity agent, Ipilimumab, throughout the titration curve. This is in contrast to the effect seen with IPI Fab and 121 Fab, where the synergistic effect was evident only at high concentrations (FIG. 6). The second pair of antibodies, BNI3 and BioE2032 (the human IgG1 version of 121), had more than 10 × the activity of either BNI3 or BioE2032, and the synergistic activity extended throughout the titration curve to the lowest dilution.

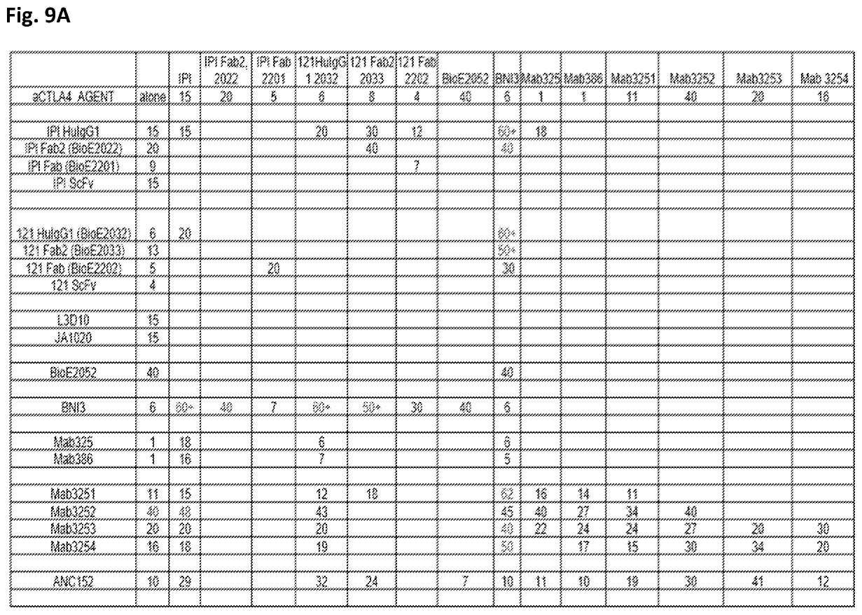

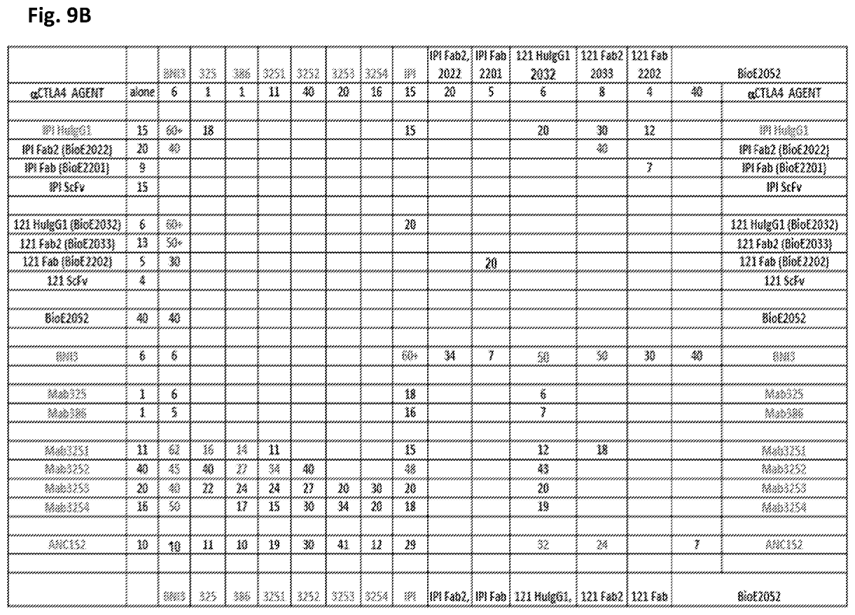

The ratios from each assay of the combinations, where the test agents were at 1000 nM, are shown in FIG. 9.

As reference activity concentration curves, Ipilimumab (human IgG1 antibody), JA1020 (human IgG1 antibody), and L3D10 (a mouse IgG1 anti-CTLA4 antibody) had titration curves that plateau at ratios of 15-20 RLU over background at 200 nM or greater (FIG. 2). These titration curves have been extended out to 2000 nM for both Ipilimumab and L3D10 individually and when in combination (4000 nM), and there was no significant increase in their ratios above 15-18.

From the review of FIG. 9 Table showing the antibody combination activity versus concentration curves, there are 4 general classes of antibody pair responses. We can parse the 4 classes of response into two general categories. First, there are pairs that do not show a change in the magnitude of the response, that is the response curve has the same shape as the either of the original activity concentration curves, but the half max activity could be shifted to a concentration intermediate between the two original concentrations, i.e., a simple average of the 2 half max concentrations. This response is typified by the addition of two antibodies that bind the same site or epitope, e.g., Ipilimumab and L3D10.

Second, there are the classes of activity titration curves that have magnitudes different from the original individual antibody activity titration curves. There are pairs where their peak activities are (i) less than additive, (ii) additive (within standard deviation), or (iii) clearly synergistic (with consideration of the standard deviation error). The less than additive situation could be due to some inhibitory effect or the antibodies interfering with each other's binding directly on the target or indirectly in that binding to one site changes the receptor tertiary structure such that its partner cannot bind. Or possibly the binding of one antibody sends a negative signal that when added to another signal results in less than either activity.

There are numerous combinations that show much higher activities than Ipilimumab or BioE2052. One of the most striking partners is BNI3. Numerous antibodies in combination with BNI3 have notable increased responses in the CTLA functional blockade assay.

BNI3 is a mouse IgG2a developed by Prof BM Broker's Laboratory at Bernhard-Nocht-Institutfiur Tropenmedizin, Hamburg, Germany, and first described in the publication Castan et al ‘97 “Accumulation of CTLA 4 expressing T lymphocytes in the germinal centers of human lymphoid tissues” Immunology 1997, 90 265-271. BNI3 is purified from a hybridoma and available from numerous reagent suppliers. BNI3 is used as a comparator in U2002/0086014A1 Human CTLA 4 antibodies and their uses. Inventors: Alan Korman, Edward L. Halk and Nils Longberg. 10D1, the precursor to Ipilimumab, is described. BNI3 appears comparable to 10D1. Although the binding site of BNI3 on CTLA4 is unknown, Zhang et al ‘19 showed that Ipilimumab did not affect BNI3 binding to CTLA 4 and could also co-bind presumably to the same CTLA4. BNI3 was sourced from BioXcell which is listed as BN13 in their catalog.

Combinations of other anti-CTLA 4 antibodies with BNI3 also led to striking synergistic response curves; the magnitude of the responses was much more than additive of the original antibodies in the pair, and this response continued throughout the titration curve. An additive or synergistic response could be suggestive of multiple pathways converging to the same output, e.g., T cell activation demonstrable by the magnitude of IL-2 expression. The combination pair of BNI3 with Ipilimumab or IPI Fab2 (BioE2022); and BNI3 with 121 HulgG1 (BioE2032) or 121 Fab2 (BioE2033) showed the most pronounced synergy and generated the highest magnitude of response in the Promega anti-CTLA4 Functional Blockade Assay.

Note also for IPI, BNI3, BioE2032, and BioE2033, the half maximum activity concentration is about 50 nM; given the resolution of the αCTLA 4 functional blockade assay (the number of titration curve samples), the half max is likely in the 10⁻¹⁰⁰ nM range for the combinations.

The Jurkat Raji CTLA4 functional blockade assay can be affected by the Fc of the antibody in a “bridging” effect described by Waight et al ‘18 due to the FcγRIIb receptors on the Raji cells. Tighter Fc binding from anti-CTLA 4 antibodies positively affects their functional blockade activity while anti-CD32 antibodies which block anti-CTLA4 antibody Fc binding will negatively impact anti-CTLA4's ability to enable maximal Jurkat cell activation. Consider the BNI3+BioE2022 combination, and the BNI3+BioE2033 combination. BNI3 is a mouse IgG2a and has poor binding to Human FcγRIIb. BioE2022 is a Fab2 version of Ipilimumab. BioE2033 is a Fab2 of BioE2032. The both combinations of (i) BNI3+BioE2022, and (ii) BNI3+BioE2033 were potent in the blockade assay, though somewhat less than the parental forms of Ipilimumab and BioE2032, despite likely poor “bridging” properties. This is relevant to development of these antibodies for therapeutic use where efficacy is weighed against toxicity.

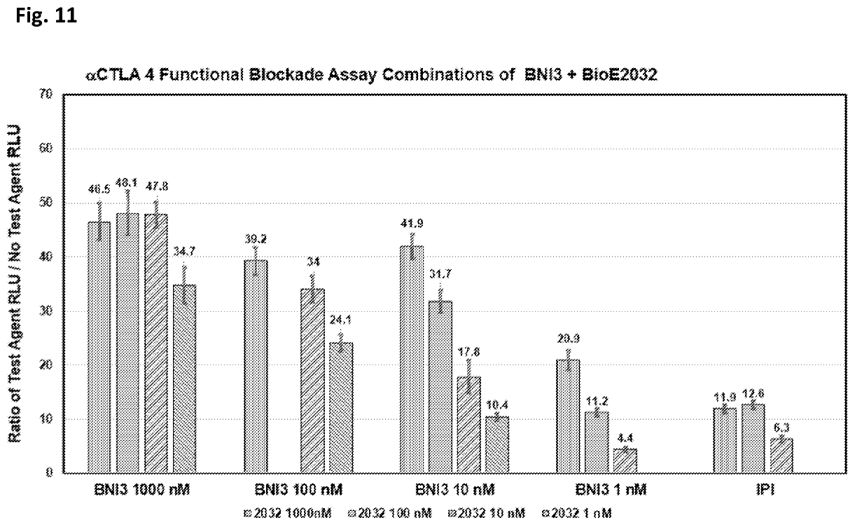

Combination pairs of BNI3 and IPI (FIG. 10); and BNI3 and BioE2032 (FIG. 11) in different concentrations ratios where one partner is fixed at a specific concentration and the other is varied, were tested. The different combination pairs were assayed for their effect in the CTLA4 functional blockade assay. As expected throughout the different concentrations, the pairs always showed a 2-3× multiple of the IPI activity. For both pairs the ratio of each pair member could be 1:100 with respect to either partner and have no effect on their biological activity; though there appears to be a minimal concentration of 10 nM for either partner while the corresponding partner be in excess of 100 nM. For the combination pairs of BNI3+BioE2032 and BNI3+IPI, the combination pairs do not need to be equimolar to enable their synergistic action.

Empirical assay data show that each of the individual and combinations of αCTLA4 antibodies generally have half max activity concentration in the 10⁻¹⁰⁰ nM range. No differences are seen in the various combination antibodies 100 nM and beyond in affecting the maximal synergistic response ie 3 × the IPI signal. (FIG. 10 and FIG. 11). In the combinations of BNI3+IPI and BNI3+BioE2032, as low as 1 nM of either antibody in the combination leads to a synergistic activity that is 1.5 × IPI at its maximum activity.

### Example 8: The High Biological Activity of Combinations of αCTLA 4 Antibodies Depends on Raji Cells

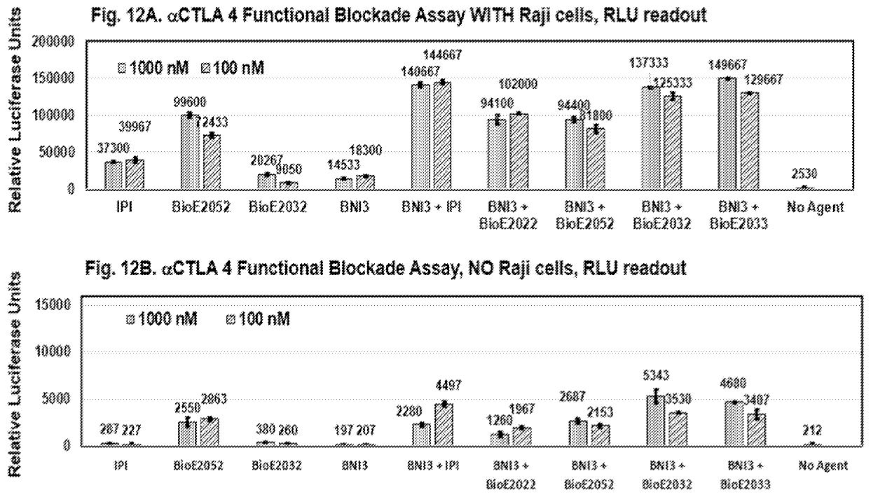

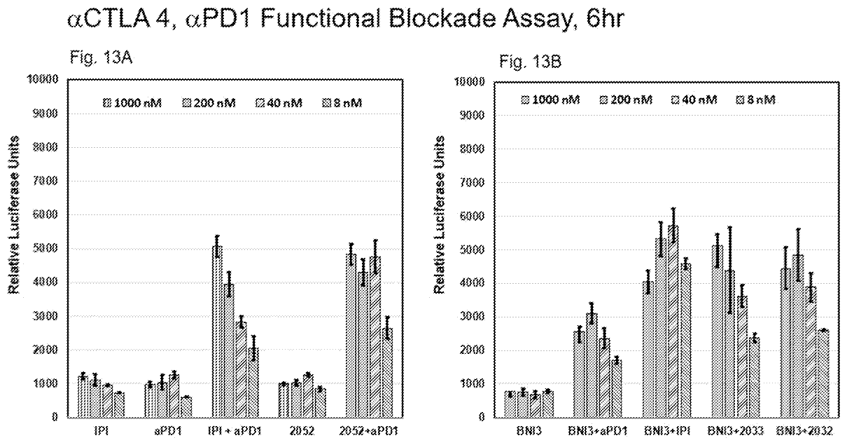

Promega's CTLA 4 functional blockade assay relies on the activation of Jurkat T cells as a first step from opposing Raji cells that express an engineered anti-CD3. The second signal of CD80, is a native cell surface ligand of Raji cells and continues the Jurkat cell activation by its native CD28. However, because the Jurkat cells have been engineered for constitutive heterologous expression of CTLA4, CTLA4 binds CD80 and aborts the activation process by depriving CD28 of ligand and possibly also by a downregulating signal. All these abrogate any expression of IL2 or the IL2 promoter driven luciferase. Antibodies such as Ipilimumab, BioE2052, and the BNI3 combinations with IPI or BioE2032 block CTLA4 from binding CD80 and also possibly convert CTLA4 into an agonistic signaling mode. Given the potent effects of BioE2052 and the BNI3 combinations, there was a possibility that the Jurkat cells alone could respond to these agents without the Raji cells. FIG. 12A and FIG. 12B show a functional blockade assay, in a conventional format with both Jurkat and Raji cells (FIG. 12A) and with Jurkat cells alone (FIG. 12B), tested with a selection of various antibodies and combinations. In FIG. 12A, the activity levels observed are consistent with previous assays, while in FIG. 12B, the parallel assay where the same set of reagents and the same Jurkat cells are used but there are no Raji cells present, the luciferase activity level is dramatically reduced. Note the one log difference in scale of the Relative Luciferase Axis in FIG. 12B compared to FIG. 12A. Without Raji cells to activate and enable CD28 driven activation, the level of luciferase, a surrogate measure of IL-2 expression, has fallen 95+% to almost the background levels. Interestingly, if the ratio of the test agent (sample) RLU is divided by the no test agent (background) RLU, there is significant stimulation above background for the highly active antibody partners. For the antibody combinations, the activity ratios vary from 12 to 25. This may be actual stimulation of stochastically activated Jurkat cells. The activities from the single type antibody, Ipilimumab, BioE2032, or BNI3, were essentially background, almost the same as no agent.

### Example 9: αCTLA 4, αPD1 Functional Blockade Assay of the αCTLA 4 Antibody Combinations

Promega has developed an αCTLA 4, αPD1 functional blockade assay using the same Jurkat Raji cell format. Specifically, the Jurkat cells are engineered to express luciferase under an NFAT promoter, while Raji cells natively express PD-L1 and CD80/86, and are engineered to express anti-CD3. When the Jurkat and Raji cells are co-cultured, the Jurkat cells are activated by anti-CD3 but the activation is quenched through both CTLA 4 and PD1 being bound to the ligand. FIG. 3 shows the output from the Jurkat cells in the Promega αCTLA 4, αPD1 assay when using either anti-CTLA4 antibody Ipilimumab or anti-PD1 antibody Nivolumab alone or in combination. When tested singularly the response curves of either Ipilimumab or Nivolumab are relatively flat almost background while when both antibodies are used together, there is a synergistic response. Presumably the marginal response of each agent alone is due to negative signaling by the other active receptor; that is negative signaling by the active PD1 receptor when bound by PD-L1 dampens the blockade of CTLA4 while negative signaling by the CD80 bound CTLA4 receptor dampens the blockade of PD1. PD1 and CTLA4 bound to ligands activate signaling pathways that converge on blocking T cell activation, that subsequently induces IL-2 expression (Willsmore et al ‘21, Wei et al ‘19, Walker ‘17.) Both CTLA4 and PD1 receptors need to be simultaneously blocked to enable continued Jurkat cell activation.

The activity of BNI3+IPI, BNI3+BioE2032, and BNI3+BioE2033 was characterized in the Promega αCTLA 4, αPD1 functional blockade assay. The results from a typical αCTLA 4, αPD1 assay incubated for 6 hours are graphed in FIG. 13A. The error bars shown are standard deviations from triplicate samples at each dilution for luciferase activity. The concentrations shown are reflective of the individual antibody concentrations. Note the control test agents of IPI or αPD1 (BioLegends) generated the expected low responses. Interestingly BioE2052 was relatively inactive alone, essentially identical to IPI or αPD1. However, the combination of BioE2052+αPD1 revealed the same maximal response as IPI+αPD1, and interestingly, the response of the titration curve showed much greater potency than IPI+αPD1. In FIG. 13B, BNI3 was a low responder in this assay while in combination with αPD1 revealed activity titration curve that is about 60% as potent as IPI+αPD1. Recall from FIG. 8, BNI3 alone is only about 30% as potent in the αCTLA 4 functional blockade and this attribute may be carried over to this assay. The more interesting results are the activity titration curves for the BNI3 combinations with IPI, BioE2033, or BioE2032. Unexpectedly, the maximal activities of these three combinations, with no αPD1, are similar to IPI+αPD1, and significantly the activity titration curves of these combinations revealed a greater potency evident in a shallow decline in activity with decreasing concentration. The PD1 receptors on the Jurkat cell are bound with PD-L1 ligand and therefore signaling to dampen the cell's activation and yet these combination antibodies are able to overcome that signaling and generate a level of activation as measured here by an IL-2 surrogate similar to simultaneous blockade of both CTLA4 and PD1.

The Promega αCTLA 4, αPD1 assay is typically incubated for 6-8 hours as prescribed in their assay manual. However, in our experimental work, we found that longer incubations are revealing. FIG. 14A and FIG. 14B are graphs from assays where the cell mixture was in culture for 20 hours. Note the readout in this assay is luciferase driven by an IL-2 type promoter. Luciferase has been engineered by Promega to have a relatively short half-life (est. 30 minutes) as a means to provide an instantaneous readout of the blockade function, and not a readout of cumulative luciferase expression.

In FIG. 14A, the reagents IPI, αPD1, and the combination of IPI+αPD1 behave as expected, consistent with what was observed in the 6 hour incubation although slightly dampened throughout. However, when the assay is incubated for 20 hrs, BioE2052 reveals an unexpected activity. BioE2052 alone showed a more potent response profile than the IPI+αPD1 combination. The entire response curve was 20% higher. The heightened response from BioE2052 is carried over to the combination of BioE2052+αPD1, whose activity profile was more than 2× that of IPI+αPD1 with a shallower titration of activity with decreasing concentration. The greater response from BioE2052 alone and in combination with αPD1 over time suggested that there might be some cellular change triggered by BioE2052 beyond its mere blockade of the CTLA4 binding action. Additionally, these changes were activating IL-2 promoter driven luciferase through a pathway that was not blocked by PD1.

In FIG. 14B, there continued to be heightened activities from the combinations of BNI3 with IPI, BioE2033, or BioE2032. The overall response profiles were very similar to what was observed at 6 hours but the overall magnitude of the response has increased about 30% throughout the titration. Note again, the very shallow titration response profile. BNI3 in combination with IPI, BioE2032, or BioE2033 shows a more potent response than either antibody as single agents or the combination of IPI and anti-PD1. The BNI3+IPI, BNI3+BioE2033, and BNI3+BioE2032 revealed a more potent response of >2 × the response observed for IPI+anti-PD1, despite presumably the negative signaling from an engaged PD1 receptor. BNI3+IPI, BNI3+BioE2033, and BNI3+BioE2032 may be activating by pathways unaffected by PDL 1 ligated PD1 receptor.

One further observation to consider is that BioE2033 is an engineered Fab2 form of BioE2032. A dimerization domain was used to make a synthetic Fab2 form of BioE2032. BioE2033 has no Fc binding capability and hence no Fc-directed functions. And as BNI3 is a mouse IgG2a antibody, there is likely little or no “bridging effect” (Waight et al ‘18) from either BNI3 or BioE2033 to Raji cells, which may affect their activity.

The addition of αPD1 or αPDL1 to combinations of BNI3+IPI or BNI3+BioE2032 further increased the magnitude of the activity titration curve 2-3× (FIG. 15A and FIG. 15B). αPD-L1 appears slightly more potent than αPD1. Note how the addition of BNI3 to a mixture of IPI+αPD1 or IPI+αPDL1 led to an almost 3× increase in the luciferase activity. The response was similar whether you blocked at the ligand or receptor. Additionally, in a parallel plate, the culture media was harvested at the termination of the assay and analyzed for IL-2 (FIG. 15B). It was evident that the IL-2 expression levels paralleled the response observed when luciferase activity was used as the readout, though in this experiment IPI (Ipilimumab) at high concentration had some effect on overcoming some of PD1 receptor antagonism.

### Example 10: The BioEntre CytoStim/LPS Stimulation of Human PBMC Assay of the αCTLA 4 Antibodies

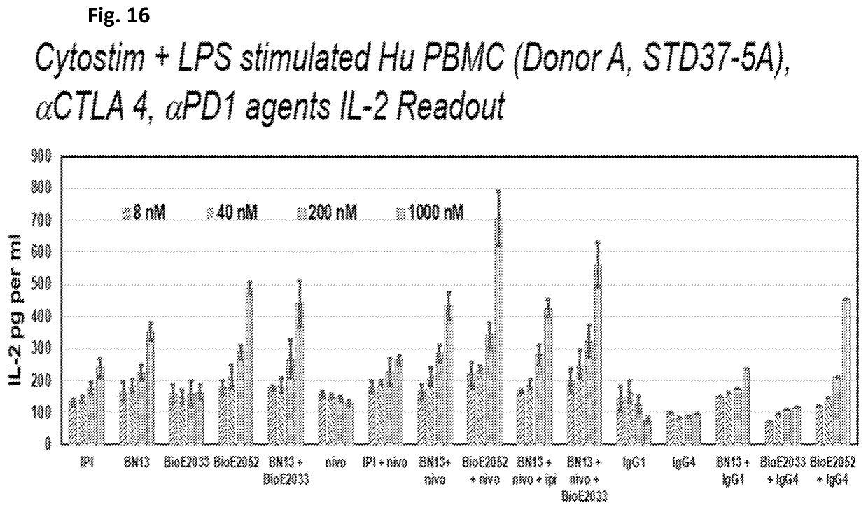

It is developed herein an assay using CytoStim™/LPS to activate primary Human T cells, PBMC, as targets to assess different αCTLA4 antibodies. FIG. 5 and FIG. 16 describe the application of this assay. Differences can be detected between different αCTLA4 antibodies and combinations.

### Example 11: Engineered αCTLA 4 Antibodies, Functional Blockade Activity Compared to Ipilimumab

It is discovered herein a series of antibodies and combinations that have differential and dynamic activity properties compared to Ipilimumab. A subset of these antibodies are described in FIG. 22. Their activities in three in vitro CTLA4 functional blockade assays were normalized to Ipilimumab. Note that individual αCTLA4 antibodies, BioE2450, BioE2460, and BioE2551, showed notably less absolute activity and specific activity than Ipilimumab and yet when in combination showed strikingly more absolute and specific activity than Ipilimumab.

### Example 12: Certain antigen-Binding Proteins for the In Vivo Studies

As described herein, certain antibody combinations have shown potent activities in the αCTLA 4 assay, and the αCTLA 4/αPD1 functional blockade assay, as well as potency in the activated the primary Hu PBMC assay (CytostimCytoStim™/LPS assay). To extend the understanding of these antibodies and their potential therapeutic applications, we proceeded to scale the manufacture of these antibodies for in vivo studies in the mouse models.

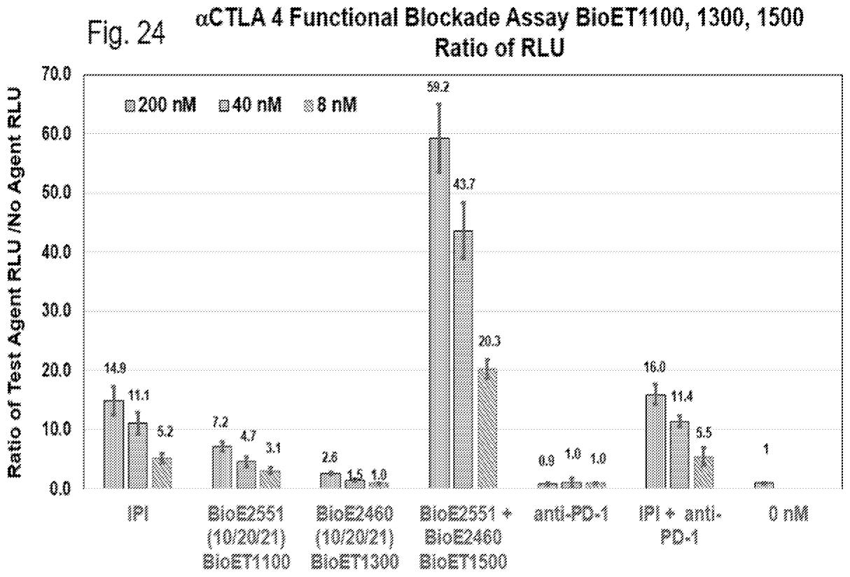

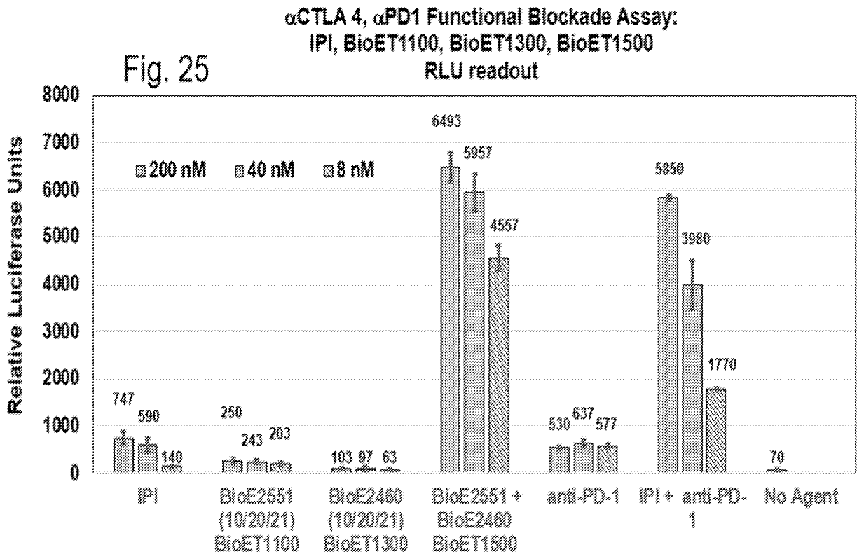

BioE2551 and BioE2460 were manufactured and purified to >95% homogeneity as determined by SEC HPLC, SDS PAGE. The purified proteins were then reformulated to 5 mg per ml in PBS as single agents; BioET1100, <0.1 EU per ml and BioET1300<0.3 EU per ml, respectively and the combination of BioE2551+BioE2460 as BioET1500. These formulations were assembled and then tested in both the Promega anti-CTLA4 functional blockade assay (FIG. 22) and in the Promega anti-CTLA4 anti-PD1 functional blockade assay (FIG. 24).

In the anti-CTLA4 anti-PD1 bioassay, both CTLA4 and PD1 are engaged by ligands CD80 and PD-L1 expressed on the Raji cells and therefore dampen the Jurkat cell activation. The lower single agent activities of Ipilimumab and anti-PD1 were expected as previously observed and so was the response when both anti-CTLA4 and anti-PD1 reagents were equimolar in a mixture. The activity of BioET1500 was also consistent with the previous observations; BioE2551+BioE2460 was a potent combination in inciting T cell activation and exceeded the activity seen with Ipilimumab+anti-PD1. The addition of anti-PD1 to BioET1500 led to a further 3+× increase in the magnitude of the titration curve (data not shown).

### Example 13: Subcutaneous H22 and Mc38 Mouse Tumor Models: Test of Bioet1100, Bioet1300, Bioet1400, and Bioet1500

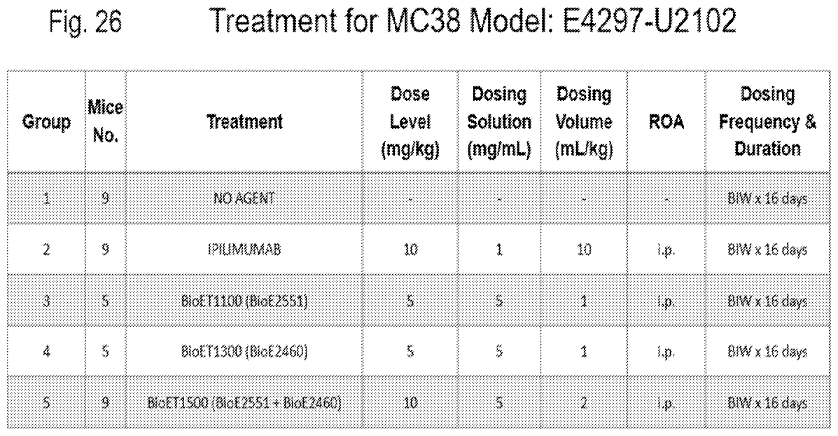

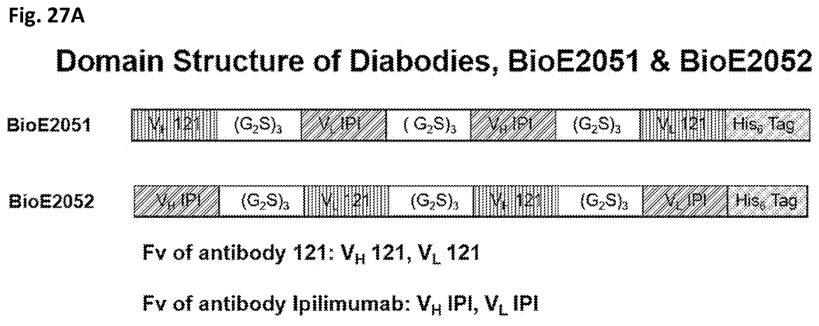

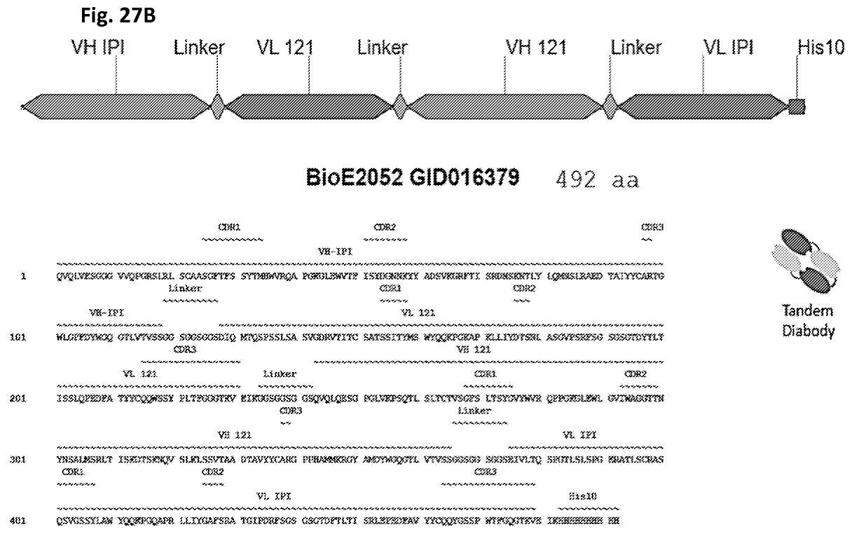

BioET1100, BioET1300, BioET1400, BioET1500, and Ipilimumab are tested (study E4297-U2102) for their tumor killing efficacy in the MC38 colorectal cancer model using humanized CTLA4 female C57Bl/6 mice. Similar study design is used to test said antigen-binding proteins in the H22 hepatocellular carcinoma model. Mice at 6-9 weeks are implanted with 10⁶ MC38 tumor cells suspended in PBS in the right lower flank. 9 days later when the tumors reached approximately 92 mm³, the mice are randomized into 5 groups and treated as shown in FIG. 26. The day of randomization is labeled as day 0.

Animals are dosed on days 0, 3, 7, 10, and day 14. Tumor sizes and body weight are measured on days 0, 2, 6, 9, 13, and on day 16 when the study was terminated.

### Example 14: BioE2052-IgG1 Fc Fusion Proteins

In an effort to increase half-life and stability of BioE2052, constructs were generated that encode BioE2052 fused to a conventional IgG1 Fc or to an IgG1 Fc with an LALAPG mutation. This mutation blocks BioE2052 binding to the Fcγ receptor, thus eliminating antibody-dependent cellular cytotoxicity (ADCC).

The addition of an albumin-binding protein to BioE2052 was also examined. Similar to above, the carboxy end of BioE2052 was fused to the amino end of mouse albumin.

### Example 15: Antigen-Binding Proteins Fused to an IgG Fc Peptide or an Albumin-Binding Proteins for Increased Stability

Two monoclonal anti-CTLA4 antigen-binding proteins (BioE2420 and BioE2430) were generated. BioE2420 comprises the amino acid sequence of BioE2052 with an Fc peptide fused to the carboxy terminal of the BioE2052 peptide. BioE2430 comprises the amino acid sequence of BioE2052 with an albumin peptide fused to the carboxy terminal of the BioE2052 peptide.

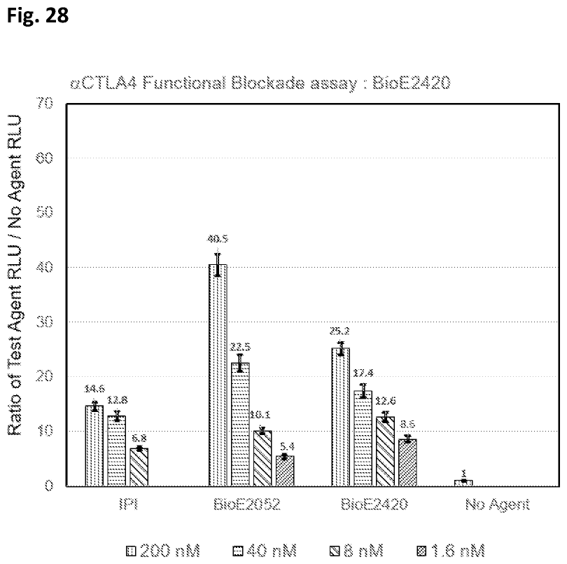

Monomers were purified by size exclusion HPLC (SEC-HPLC). The activity of the purified monomers was assessed using the CTLA4 Functional Blockade Assay. As shown in FIG. 28, BioE2420 had slightly less anti-CTLA4 activity relative to BioE2052, but more than Ipi.

### Example 16: Extending Half-Life In Vivo

To improve or modify the pharmacokinetic (PK) properties of the antigen-binding proteins described herein, the antigen-binding domains (e.g., BioE2052) are modified (e.g., fused) to comprise a PK modulator. The modified antigen-binding protein is administered to MC38 model mice according to the schedule shown in FIG. 26. Blood samples are drawn at regular intervals and assayed for the presence of the modified antigen-binding protein to determine the bioavailability of the modified antigen-binding protein. Results are compared to those observed in MC38 mice that are administered vehicle (e.g., saline), those that are administered ipilimumab, and those that are administered only the antigen-binding protein (i.e., no Fc domain). Results show an improvement in the bioavailability of the modified antigen-binding protein relative to the normal control group and the group that are administered only the antigen binding protein. Additionally, survivability is improved in mice administered the modified antigen-binding protein relative to the normal control group and the group that are administered only the antigen binding protein.

Tumor volume and growth are measured in the MC38 mice that are administered the modified antigen-binding protein and are compared to tumor volume and growth observed in MC38 mice that are administered only vehicle (e.g., saline), MC38 mice that are administered ipilimumab, and MC38 mice that are administered only the antigen-binding protein. Results show an improvement in tumor volume and growth in mice receiving the modified antigen-binding protein relative to the normal control group and the group administered only the antigen binding protein.

### REFERENCES

Bauche D, Mauze S, Kochel C, Grein J, Sawant A, Zybina Y, Blumenschein W, Yang P, Annamalai L, Yearley J H, Punnonen J, Bowman E P, Chackerian A and Laface D 2020 Antitumor efficacy of combined CTLA4/PD-1 blockade without intestinal inflammation is achieved by elimination of FcγR interactions J. Immunother Cancer 2020; 8: e001584
Castan J, Tenner-Racz K, Racz P, Fleischer B and Broker B M 1997 Accumulation of CTLA-4 expressing T lymphocytes in the germinal centres of human lymphoid tissues Immunology 90:265-271
Chen W, Pandey M, Sun H, Rolong A, Co M, Liu D, Wang J, Zeng L, Hunter and Lin S 2021 Development of a mechanism of action-reflective, dual target cell-based reporter bioassay for a bispecific monoclonal antibody targeting human CTLA4 and PD-1 MABS 13 (1) 1914359
Cong M, Cheung Z-J J, Stecha P, Wang J, Grailer J, Karassina N, Harnett J and Fan F 2015 Reporter Bioassays to Assess Therapeutic Antibodies in Development for Immunotherapy Programs
Du X, Liu M, Su J, Zhang P, Tang F, Ye P, Devenport M, Wang X, Zhang Y, Liu Y and Zheng P. 2018 Uncoupling therapeutic from immunotherapy-related adverse effects for safer and effectiuve anti-CTLA 4 antibodies in CTLA humanized mice. Cell Research (2018) 4:433-447
Gombos R B, Gonzalez A, ManriqueM, Chand D, Savitsky D, Morin B, Breous-Nysgtrom E, Dupont C, Ward R A, Mundt C, Duckless B, Tang H, Findeis M A, Schuster A, Wright J D, Underwood D, Clarke C, Ritter G, Merghoub T, Schaer D, Wolchok J D, van Dijk M,
Buell J S, Cuillerot J-M, Stein R, Drouin E E, and Wilson N S. 2018 Toxicological and pharmacological assessment of AGEN1884, a novel human IgG1 anti-CTLA4 antibody PloS ONE 13 (4): e0191926
Ha D, Tanaka A, Kibayashi T, Tanemura A, Sugiyama D, Wing J B, Lim E L, Teng KWW, Adeegbe D, Newell E W, Katayama I, Nishikawa H and Sakaguchi S 2019 Differential control of human Treg and effector T cells in tumor immunity by Fc-engineered anti-CTLA 4 antibody PNAS 116 (2): 609-618
Hill A V 1910 The possible effects of the aggregation of the molecules of huemoglobinon its dissociation curves. Proceeding of the Physiological Society page iv-v
Ingram J R, Blomberg O S, Rashidian M, Ali L, Garforth S, Fedorov E, Fedorov A A, Bonanno J B, Le Gall C, Crowley S, Espinosa C, Biary T, Keliher E J, Weissleder R, Almo S C, Dougan S K, Ploegh H L, and Dougan M. 2018 Anti-CTLA4 therapy requires an Fc domain for efficacy PNAS 115 (15): 3212-3917
Jutz S, Hennig A, Paster W, Asrak O, Dijanovic D, Kellner F, Pickl W F, Huppa J B, Leitner J and Steinberger P 2017 A cellular platform for the evaluation of immune checkpoint molecules Oncotarget 8 (39): 64892-64906
Liu C, Yu C, Yang Y, Cui Y, Zhang F, Wang L, Wang J 2021 Development and validation of a reporter gene assay to determine the bioactivity of anti-CTLA4 monoclonal antibodies International Immunopharmacology 101 (Pt A): 108277
Lund J, Winter G, Jones P T, Pound J D, Tanaka T, Walker M R, Artymiuk P J, Arata Y, Burton D R, Jefferis R and Woof J M 1991 Human Fc gamma R I and Fc gamma RII interact with distinct but overlapping sites on human IgG. J Immunol 147 (8): 2657-2662
May K F, Roychowdhury S, Bhatt D, Kocak E, Bai X-F, Liu J-Q, Ferketich A K, Martin E W, Caligiuri M A and Liu Y 2005, Anti-human CTLA4 monoclonal antibody promotes T-cell expansion and immunity in a hu-PBL-SCID model: a new method for preclinical screening of costimulatory monoclonal antibodies Blood 105 (3): 1114-1120
Quezada S A and Peggs K S 2019 Lost in Translation: Deciphering the mechanism of action of anti-human CTLA-4 Clin Cancer Res 2019 25 (4): 1130-1132
Schlothauer t, Herter S, Koller C F, Grau-Richards S, Steinhart V, Spick C, Kubbies M, Klein/c, Umana P and Mossner E 2016 Novel human IgG1 and IgG4 Fc-engineered antibodies with completely abolished immune effector functions Protein Engineering 29 (10): 457-466
Schofield D J, Percival-Alwyn J, Rytelewski M, Hood J, Rothstein R, Wetzel L, McGlinchey K, Adjel G, Watkins A, Machiesky L-A, Chen W, Andrews J, Groves M, Marrow M, Stewart R A, Leinster A, Wilkinson R W, Hammond S A, Luheshi N, Dobson C and Oberst M 2021 Activity of murine surrogate antibodies for durvalumab and tremelimumab lacking effector function and the ability to deplete regulatory T cells in mouse models of cancer MABS 13 (1), e1857100
Sharma A, Subudhi S K, Blando J, Scutti J, Vence L, Wargo J, Allison J P, Ribas A and Sharma P 2018 Anti-CTLA4 Immunotherapy Does Not Deplete FOXP3 ˜ Regulatory T Cells (Tregs) in Human Cancers Clin Cancer Res 2019 25 (4): 1233-1238
Simpson T R, Fubin L, Montalvo-Ortiz W, Sepulveda M A, Bergerhoff K, Arce F, Roddie C, Henry J Y, Yagita H, Wolchok J D, Peggs K S, Ravetch J V, Allison J P and Quezada S A 2013 Fc-dependent depletion of tumor-infiltrating regulatory T cells co-defines the efficacy of anti-CTLA4 therapy against melanoma 2013 J. Exp. Med. 2013 Vol. 210, 9:1695-1710
Stone E L, Carter K P, Wagner E K, Asensio M A, Benzie E, Chiang Y Y, Coles G L, Edgar C, Gautam B K, Gras A, Leong J, Leong R, Manickam V A, Mizrahi R A, Niedecken A R, Saini J, Sandhu S K, Simons J F, Stadtmiller K, Tinsley B, Tracy L, Wayham N P, Lim Y W, Adler A S and Johnson D S 2021 Lack of blocking activity in anti-CTLA 4 antibodies reduces toxicity but not anti-tumor efficacy
Vargas F A, Furness AJS, Litchfield K, Joshi K, Rosenthal R, Ghorani E, Solomon I, Lesko M H, Ruef N, Roddie C, Henry J Y, Spain L, Aissa A B, Georgiou A, Wong YNS, Smith M, Strauss D, Hayes A, Nicol D, O'Brien T, Martesnsson L, Ljungars A, Teige I, Frende B, TRACERx Melanoma, TRACERx Renal, TRACERx Lung consortia, Pule M, Marafioti T, Gore M, Larkin J, Turailic S, Swanton C, Peggs K S and Quezada S A 2018 Fc Effector Function Contributes to the Activity of Human Anti-CTLA4 Antibodies Cancer Cell 33:1-15
Waight J D, Chand D, Dietrich S, Gombos R, Horn T, Gonzalez A M, Manrique M, Swiech L, Morin B, Brittsan C, Tanne A, Akpeng B, Croker B A, Buell J S, Stein R, Savitsky D A, and Wilson N S 2018 Selective FcgR Co-engagement on APCs Modulates the Activity of Therapeutic Antibodies Targeting

### Cell Antigens Cancer Cell 33, 1033-1047

Walker LSK 2017 PD-1 and CTLA4: Two checkpoints, one pathway. Sci. Immunol eaan3864 (2017)
Walker LSK and Sansom D M 2015 Confusing signals: Recent progress in CTLA-4 Biology Trends in Immunology 36 (2): 63-70
Wei S C, Levine J H, Cogdill A P, Zhao Y, Anang N-A A S, Andrews M C, Sharma P, Wang J, Wargo J A, Pe′er D and Allison J P 2017 Distinct Cellular Mechanisms Underlie Anti-CTLA4 and Anti-PD-1 Checkpoint Blockade. Cell 170:1120-1133
Willsmore Z N, Coumbe BGT, Crescioli S, Reci S, Gupta A, Harris R J, Chenoweth A, Chauhan J, Bax H J, McCraw A, Cheung A, Osborn G, Hoffman R M, Nakamura M, Laddach R, Geh JLC, Mackenzie-Ross A, Healy C, Tsoka S, Spicer J F, Josephs D H, Papa
S, Lacy K E and Karagiannis S N 2022 Combined anti-PD-1 and anti-CTLA4 checkpoint blockade: Treatment of melanoma and immune mechanisms of action Eur. J. Immunol 51:544-556
Xu W, Cummings J, Sank M, Juhel M, Li X, Gleason C, DeSilva B S, Dodge R W, and Pillutla R 2018 Development and validation of a functional cell-based neutralizing antibody assay for ipilimumab Bioanalysis 10 (16): 1273-1287
Yang L, Wang J, Cheke R A and Tang S A 2021 A Universal Delayed Difference Model fitting dose-response curves Dose-Response 2021:1-18
Yordanov P and Stelling J 2018 Steady-State Differential Dose Response in Biological Systems Biophysical Journal 114:723-736

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

What is claimed is:

1. An antigen-binding protein, comprising:
a) a heavy chain variable domain (VH) amino acid sequence set forth in Table 4, or a variant sequence thereof which differs by only one or two amino acids or which has at least or about 85% sequence identity; and/or
b) a light chain variable domain (VL) amino acid sequence set forth in Table 4, or a variant sequence thereof which differs by only one or two amino acids or which has at least or about 85% sequence identity.

2. The antigen-binding protein of claim 1, wherein the antigen-binding protein comprises the VH and VL amino acid sequences set forth in:
a) SEQ ID NO: 72 and SEQ ID NO: 73;
b) SEQ ID NO: 74 and SEQ ID NO: 75;
c) SEQ ID NO: 76 and SEQ ID NO: 77;
d) SEQ ID NO: 78 and SEQ ID NO: 79;
e) SEQ ID NO: 80 and SEQ ID NO: 81;
f) SEQ ID NO: 82 and SEQ ID NO: 83;
g) SEQ ID NO: 84 and SEQ ID NO: 85;
h) SEQ ID NO: 86 and SEQ ID NO: 87;
i) SEQ ID NO: 88 and SEQ ID NO: 89;
j) SEQ ID NO: 90 and SEQ ID NO: 91;
k) SEQ ID NO: 92 and SEQ ID NO: 93;
l) SEQ ID NO: 94 and SEQ ID NO: 95;
m) SEQ ID NO: 96 and SEQ ID NO: 97;
n) SEQ ID NO: 98 and SEQ ID NO: 99;
o) SEQ ID NO: 100 and SEQ ID NO: 101;
p) SEQ ID NO: 102 and SEQ ID NO: 103;
q) SEQ ID NO: 104 and SEQ ID NO: 105; or
a variant sequence thereof which differs by only one or two amino acids or which has at least or about 85% sequence identity.

3. The antigen-binding protein of claim 1 or 2, wherein the antigen-binding protein
a) binds specifically to CTLA4; and/or
b) blocks the interaction between CTLA4 and its ligands (e.g., CD80 (B7-1) and CD86 (B7-2)),
optionally further comprising a heterologous sequence.

4. The antigen-binding protein of any one of claims 1-3, wherein the antigen-binding protein does not comprise an Fc domain.

5. The antigen-binding protein of any one of claims 1-4, wherein the antigen-binding protein comprises an Fc domain, optionally wherein the Fc domain is a human IgG1 Fc.

6. The antigen-binding protein of any one of claims 1-3 and 5, wherein the antigen-binding protein comprises an immunoglobulin heavy chain constant domain selected from the IgG, IgG1, IgG2, IgG2A, IgG2B, IgG3, IgG4, IgA, IgM, IgD, and IgE constant domains.

7. The antigen-binding protein of any one of claims 1-6, wherein the antigen-binding protein does not bind to one or more Fc receptors.

8. The antigen-binding protein of any one of claims 5-7, wherein the Fc domain comprises a LALAPG mutation or a LALA mutation.

9. The antigen-binding protein of any one of claims 1-8, wherein the antigen-binding protein is selected from an antibody, Fv, F(ab′)2, Fab′, dsFv, scFv, sc(Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

10. The antigen-binding protein of any one of claims 1-9, wherein the antigen-binding protein is an antibody, optionally a monoclonal antibody.

11. The antigen-binding protein of any one of claims 1-10, wherein the antigen-binding protein is chimeric, humanized, composite, murine, or human, optionally wherein the antigen-binding protein is humanized.

12. The antigen-binding protein of any one of claims 1-11, wherein the antigen-binding protein is:
a) an IgG1 monoclonal antibody; or
b) an IgG1 monoclonal antibody comprising a LALAPG mutation in the Fc region.

13. The antigen-binding protein of any one of claims 1-12, wherein the antigen-binding protein is conjugated or detectably labeled.

14. The antigen-binding protein of any one of claims 1-13, wherein the antigen-binding protein further comprises a polymer or a heterologous polypeptide.

15. The antigen-binding protein of any one of claims 1-14, wherein the heterologous polypeptide comprises a peptide tag (e.g., His6 tag) and/or a leader sequence.

16. The antigen-binding protein of claim 14 or 15, wherein the polymer or the heterologous polypeptide extends a half-life of the antigen-binding protein.

17. The antigen-binding protein of any one of claims 14-16, wherein the heterologous polypeptide comprises an albumin-binding protein, albumin, an Fc domain, a fragment of an Fc domain, an FcRnBP.

18. The antigen-binding protein of claim 14 or 16, wherein the polymer comprises polyethylene glycol (PEG) or a variant thereof (e.g., glycol-PEG).

19. An isolated nucleic acid that encodes the antigen-binding protein of any one of claims 1-18.

20. A vector comprising the isolated nucleic acid of claim 19.

21. A host cell which comprises the isolated nucleic acid of claim 19, comprises the vector of claim 20, and/or expresses the antigen-binding protein of any one of claims 1-18.

22. A pharmaceutical composition comprising the antigen-binding protein of any one of claims 1-18, an isolated nucleic acid of claim 19, a vector of claim 20, and/or a host cell of claim 21.

23. The pharmaceutical composition of claim 22, further comprising an antigen-binding protein that binds PD-1 and/or PD-L1.

24. The pharmaceutical composition of claim 23, wherein the antigen-binding protein that binds PD-1 or PD-L1 is selected from nivolumab, pembrolizumab, atezolizumab, avelumab, durvalumab, cemiplimab, and dostarlimab.

25. An antigen-binding protein comprising an Fc domain, wherein the antigen-binding protein does not bind to one or more Fc receptors, and comprises the VH and VL domain amino acid sequences set forth in:
a) SEQ ID Nos: 12 and 14;
b) SEQ ID Nos: 15 and 17; or
a variant sequence thereof which differs by only one or two amino acids or which has at least or about 85% sequence identity.

26. The antigen-binding protein of claim 25, wherein the antigen-binding protein
a) binds specifically to CTLA4; and/or
b) blocks the interaction between CTLA4 and its ligands (e.g., CD80 (B7-1) and CD86 (B7-2)).

27. The antigen-binding protein of claim 25 or 26, wherein the antigen-binding protein comprises an immunoglobulin heavy chain constant domain selected from the IgG, IgG1, IgG2, IgG2A, IgG2B, IgG3, IgG4, IgA, IgM, IgD, and IgE constant domains.

28. The antigen-binding protein of any one of claims 25-27, wherein the antigen-binding protein is an antibody, optionally a monoclonal antibody.

29. The antigen-binding protein of any one of claims 25-28, wherein the antigen-binding protein is chimeric, humanized, composite, murine, or human, optionally wherein the antigen-binding protein is humanized.

30. The antigen-binding protein of any one of claims 25-29, wherein the Fc domain comprises a LALAPG mutation or a LALA mutation.

31. The antigen-binding protein of any one of claims 25-30, wherein the antigen-binding protein is a human IgG1 monoclonal antibody comprising a LALAPG mutation in the Fc region.

32. The antigen-binding protein of any one of claims 25-31, wherein the antigen-binding protein is conjugated or detectably labeled.

33. The antigen-binding protein of any one of claims 25-32, wherein the antigen-binding protein further comprises a polymer or a heterologous polypeptide.

34. The antigen-binding protein of claim 33, wherein the heterologous polypeptide comprises a peptide tag (e.g., His6 tag) and/or a leader sequence.

35. The antigen-binding protein of claim 33 or 34, wherein the polymer or the heterologous polypeptide extends a half-life of the antigen-binding protein.

36. The antigen-binding protein of any one of claims 33-35, wherein the heterologous polypeptide comprises an albumin-binding protein, albumin, a fragment of an Fc domain, or an FcRnBP.

37. The antigen-binding protein of claim 33 or 35, wherein the polymer comprises polyethylene glycol (PEG) or a variant thereof (e.g., glycol-PEG).

38. A pharmaceutical composition comprising the antigen-binding protein of any one of claims 25-37.

39. The pharmaceutical composition of claim 38, further comprising an antigen-binding protein that binds PD-1 and/or PD-L1.

40. A pharmaceutical composition comprising an antigen-binding protein that binds PD-1 or PD-L1; and an antigen-binding protein comprising the VH and VL amino acid sequences set forth in:
a) SEQ ID NOs: 12 and 14;
b) SEQ ID NOs: 12, 14, 15, and 17;
c) SEQ ID NO: 24, or
a variant sequence thereof which differs by only one or two amino acids or which has at least or about 85% sequence identity.

41. The pharmaceutical composition of claim 39 or 40, wherein the antigen-binding protein that binds PD-1 or PD-L1 is selected from nivolumab, pembrolizumab, atezolizumab, avelumab, durvalumab, cemiplimab, and dostarlimab.

42. A composition comprising at least two antigen-binding proteins that specifically bind cytotoxic T-lymphocyte-associated antigen-4 (CTLA4).

43. The composition of claim 42, wherein the at least two antigen-binding proteins bind different epitopes of CTLA4.

44. The composition of claim 43, wherein the epitope of CTLA4 is selected from the residues 134MYPPPY139, the residues 65SICT68, and the residues 58ELT60 of CTLA4.

45. The composition of any one of claims 42-44, wherein the at least one antigen-binding protein blocks the interaction between CTLA4 and its ligands (e.g., CD80 (B7-1) and CD86 (B7-2)).

46. The composition of any one of claims 42-45, wherein at least one antigen-binding protein does not comprise an Fc domain.

47. The composition of any one of claims 42-46, wherein at least one antigen-binding protein comprises an Fc domain, optionally wherein the Fc domain is a human IgG1 Fc.

48. The composition of any one of claims 42-45 and 47, wherein at least one antigen-binding protein comprises an immunoglobulin heavy chain constant domain selected from the IgG, IgG1, IgG2, IgG2A, IgG2B, IgG3, IgG4, IgA, IgM, IgD, and IgE constant domains.

49. The composition of any one of claims 42-48, wherein at least one antigen-binding protein does not bind to one or more Fc receptors.

50. The composition of any one of claims 42-49, wherein at least one antigen-binding protein comprises the Fc domain comprising a LALAPG mutation or a LALA mutation.

51. The composition of any one of claims 42-50, wherein at least one antigen-binding protein is selected from an antibody, Fv, F(ab′)2, Fab′, dsFv, scFv, sc (Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

52. The composition of any one of claims 42-51, wherein at least one antigen-binding protein is an antibody, optionally a monoclonal antibody.

53. The composition of any one of claims 42-52, wherein at least one antigen-binding protein is chimeric, humanized, composite, murine, or human, optionally wherein the antigen-binding protein is humanized.

54. The composition of any one of claims 42-53, wherein at least one antigen-binding protein is:
a) an IgG1 monoclonal antibody; or
b) an IgG1 monoclonal antibody comprising a LALAPG mutation in the Fc region.

55. The composition of any one of claims 42-54, wherein at least one antigen-binding protein is conjugated or detectably labeled.

56. The composition of any one of claims 42-55, wherein at least one antigen-binding protein further comprises a polymer or a heterologous polypeptide.

57. The composition of claim 56, wherein the heterologous polypeptide comprises a peptide tag (e.g., His6 tag) and/or a leader sequence.

58. The composition of claim 56 or 57, wherein the polymer or the heterologous polypeptide extends a half-life of the antigen-binding protein.

59. The composition of any one of claims 56-58, wherein the heterologous polypeptide comprises an albumin-binding protein, albumin, or an Fc domain.

60. The composition of claim 56 or 58, wherein the polymer comprises polyethylene glycol (PEG) or a variant thereof (e.g., glycol-PEG).

61. The composition of any one of claims 42-60, wherein at least one antigen-binding protein is selected from the antigen-binding proteins of any one of claims 1-18, 25-37, and 40.

62. The composition of any one of claims 42-61, wherein at least two antigen-binding proteins are selected from the antigen-binding proteins of any one of claims 1-18, 25-37, and 40.

63. The composition of any one of claims 42-62, wherein at least one antigen-binding protein is selected from the antigen-binding proteins listed in Table 6 and/or Table 7.

64. The composition of any one of claims 42-63, wherein at least two antigen-binding proteins are selected from the antigen-binding proteins listed in Table 6 and/or Table 7.

65. The composition of any one of claims 42-64, wherein the composition comprises:
a) an antigen-binding protein comprising the VH and VL amino acid sequences selected from SEQ ID NOs: 72-105;
b) an antigen-binding protein comprising the VH and VL amino acid sequences set forth in SEQ ID NOs: 12 and 14;
c) an antigen-binding protein comprising the VH and VL amino acid sequences set forth in SEQ ID NOs: 15 and 17;
d) an antigen-binding protein comprising the VH and VL amino acid sequences set forth in SEQ ID NOs: 12, 14, 15, and 17;
e) an antigen-binding protein comprising the sequence set forth in SEQ ID NO: 24; or
f) two antigen-binding proteins comprising any combination of a)-e).

66. The composition of any one of claims 42-65, wherein composition comprises:
a) an antigen-binding protein comprising the VH and VL amino acid sequences selected from SEQ ID NOs: 72-105; and
b) an antigen-binding protein comprising the VH and VL amino acid sequences set forth in SEQ ID NOs: 12 and 14.

67. The composition of any one of claims 42-65, wherein composition comprises:
a) an antigen-binding protein comprising the VH and VL amino acid sequences selected from SEQ ID NOs: 72-105; and
b) an antigen-binding protein comprising the VH and VL amino acid sequences set forth in SEQ ID NOs: 15 and 17.

68. The composition of any one of claims 42-65, wherein composition comprises:
a) an antigen-binding protein comprising the VH and VL amino acid sequences selected from SEQ ID NOs: 72-105; and
b) an antigen-binding protein comprising the VH and VL amino acid sequences set forth in SEQ ID NOs: 12, 14, 15, and 17; or an antigen-binding protein comprising the sequence set forth in SEQ ID NO: 24.

69. The composition of any one of claims 42-68, wherein at least one antigen-binding protein is selected from an antibody, an antibody comprising a LALAPG mutation or a LALA mutation in its Fc domain, F(ab′)2, and Fab′.

70. The composition of any one of claims 42-69, wherein the composition comprises two antigen-binding proteins that specifically bind CTLA4.

71. The composition of claim 70, wherein the two antigen-binding proteins are present at an equimolar concentration, or not present at an equimolar concentration.

72. The composition of claim 70 or 71, wherein the molar ratio of one antigen-binding protein to another antigen-binding protein is at least or about 1:1000, 1:100, 1:10, or 1:1.

73. The composition of any one of claims 42-72, further comprising an antigen-binding protein that binds PD-1 and/or PD-L1.

74. The composition of claim 73, wherein the antigen-binding protein that binds PD-1 or PD-L1 is selected from nivolumab, pembrolizumab, atezolizumab, avelumab, durvalumab, cemiplimab, and dostarlimab.

75. A pharmaceutical composition comprising the composition of any one of claims 42-74.

76. A kit comprising an antigen-binding protein of any one of claims 1-18 and 25-37; a composition of any one of claims 42-74; or a pharmaceutical composition of any one of claims 22-24, 38-41, and 75.

77. A method of producing the antigen-binding protein of any one of claims 1-18 and 25-37, wherein the method comprises the steps of: (i) culturing a host cell comprising a nucleic acid comprising a sequence encoding the antigen-binding protein of any one of claims 1-18 and 25-37 under conditions suitable to allow expression of said antigen-binding protein; and (ii) recovering the expressed antigen-binding protein.

78. A method of preventing or treating a subject afflicted with a cancer, the method comprising administering to the subject at least one selected from: an antigen-binding protein of any one of claims 1-18 and 25-37; a composition of any one of claims 42-74; and a pharmaceutical composition of any one of claims 22-24, 38-41, and 75.

79. A method of inhibiting proliferation of a cancer cell in a subject, the method comprising administering to the subject at least one selected from: an antigen-binding protein of any one of claims 1-18 and 25-37; a composition of any one of claims 42-74; and a pharmaceutical composition of any one of claims 22-24, 38-41, and 75.

80. The method of claim 78 or 79, wherein if two or more of antigen-binding proteins, compositions, or pharmaceutical compositions are administered to the subject, then the two or more of antigen-binding proteins, compositions, or pharmaceutical compositions are administered conjointly to the subject.

81. The method of claim 80, wherein an antigen-binding protein that binds PD-1 or PD-L1 is administered to the subject conjointly with:
a) an antigen-binding protein of any one of claims 1-18 and 25-37; and/or
b) an antigen-binding protein comprising the sequences set forth in:
i) SEQ ID NOs: 12 and 14;
ii) SEQ ID NOs: 12, 14, 15, and 17;
iii) SEQ ID NO: 24, or
iv) a variant sequence of any one of i)-iii) which differs by only one or two amino acids or which has at least or about 85% sequence identity.

82. The method of claim 80, wherein the subject is administered conjointly with two or more of antigen-binding proteins selected from:
a) an antigen-binding protein comprising the VH and VL amino acid sequences selected from SEQ ID NOs: 72-105;
b) an antigen-binding protein comprising the VH and VL amino acid sequences set forth in SEQ ID NOs: 12 and 14;
c) an antigen-binding protein comprising the VH and VL amino acid sequences set forth in SEQ ID NOs: 15 and 17;
d) an antigen-binding protein comprising the VH and VL amino acid sequences set forth in SEQ ID NOs: 12, 14, 15, and 17; and
e) an antigen-binding protein comprising the sequence set forth in SEQ ID NO: 24.

83. The method of claim 82, wherein the subject is further administered conjointly with an antigen-binding protein that binds PD-1 or PD-L1.

84. The method of claim 81 or 83, wherein the antigen-binding protein that binds PD-1 or PD-L1 is selected from nivolumab, pembrolizumab, atezolizumab, avelumab, durvalumab, cemiplimab, and dostarlimab.

85. The method of any one of claims 78-84, wherein the antigen-binding protein, the composition, or the pharmaceutical composition (a) decreases the number of proliferating cancer cells; (b) reduces the volume or size of a tumor of the cancer; (c) increases the immune response against the cancer; and/or (d) activates a T cell.

86. The method of any one of claims 78-84, further comprising conjointly administering to the subject an additional cancer therapy.

87. The method of claim 86, wherein the additional cancer therapy is selected from the group consisting of immunotherapy, checkpoint blockade, cancer vaccines, chimeric antigen receptors, chemotherapy, radiation, target therapy, and surgery, optionally wherein the additional cancer therapy is checkpoint blockade.

88. The method of any one of claims 78-87, wherein the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma, thyroid cancer, gall bladder cancer, head-and-neck (squamous) cancer, stomach (gastric) cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

89. The method of any one of claims 78-88, wherein the cancer is selected from melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, and B-cell lymphoma.

90. A method of increasing an immune response in a subject, the method comprising administering to the subject at least one selected from: an antigen-binding protein of any one of claims 1-18 and 25-37; a composition of any one of claims 42-74; and a pharmaceutical composition of any one of claims 22-24, 38-41, and 75.

91. A method of activating a T cell, the method comprising contacting the T cell with at least one selected from: an antigen-binding protein of any one of claims 1-18 and 25-37; a composition of any one of claims 42-74; or a pharmaceutical composition of any one of claims 22-24, 38-41, and 75.

92. A method of preventing or treating a disease or a condition characterized by aberrant expression or activity of a CTLA4 protein in a subject, the method comprising administering to the subject at least one selected from an antigen-binding protein of any one of claims 1-18 and 25-37; a composition of any one of claims 42-74; or a pharmaceutical composition of any one of claims 22-24, 38-41, and 75.

93. The method of claim 92, wherein the disease or condition is a cancer, autoimmune disease, infection, or inflammatory disease.

94. The method of any one of claims 78-93, wherein the subject is a mammal, optionally a mouse, a dog, or a cat, or a human.
